(19) United States
(12) Patent Application Publication
(10) Pub. No.: US 2022/0160713 A1
(43) Pub. Date: May 26, 2022

(54) SELECTIVE INHIBITOR OF PROTEIN ARGININE METHYLTRANSFERASE 5 (PRMT5)

(71) Applicant: PRELUDE THERAPEUTICS, INCORPORATED, Wilmington, DE (US)

(72) Inventors: Hong LIN, Exton, PA (US); Qun LI, Newark, DE (US); Mark ANDRES, West Lafayette, IN (US); Huaping ZHANG, Newark, DE (US)

(21) Appl. No.: 17/430,815

(22) PCT Filed: Feb. 13, 2020
(86) PCT No.: PCT/US2020/018185

§ 371 Date: Aug. 13, 2021

Related U.S. Application Data
(60) Provisional application No. 62/805,175, filed on Feb. 13, 2019.
(60) Provisional application No. 62/805,726, filed on Feb. 14, 2019.

(51) Int. Cl. A61K 31/519; A61K 45/06; A61P 35/02

(52) U.S. Cl. CPC A61K 31/519; A61P 35/02; A61K 45/06

(57) ABSTRACT

The disclosure is directed to crystalline forms of the compound of Formula I, pharmaceutically acceptable salts of the compound of Formula I, and crystalline forms thereof. Pharmaceutical compositions comprising said crystalline forms and salts, as well as methods of their use and preparation, are also described.

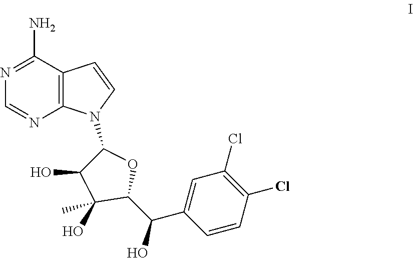 [SMILES: C[C@@]1(O)[C@@H]([C@H](O)c2ccc(Cl)c(Cl)c2)O[C@@H](n2ccc3c(N)ncnc32)[C@@H]1O]

### CROSS-REFERENCE TO RELATED APPLICATIONS

This application claims the benefit of priority to U.S. Provisional Patent Application No. 62/805,175 filed Feb. 13, 2019 and U.S. Provisional Patent Application No. 62/805,726 filed Feb. 14, 2019. Each of these applications is incorporated by reference herein in its entirety.

### TECHNICAL FIELD

The disclosure is directed to PRMT5 inhibitors and methods of their use.

### BACKGROUND

Protein arginine methylation is a common post-translational modification that regulates numerous cellular processes, including gene transcription, mRNA splicing, DNA repair, protein cellular localization, cell fate determination, and signaling. Three types of methyl-arginine species exist: ω NG monomethylarginine (MMA), ω NG, NG asymmetric dimethylarginine (ADMA) and ω NG, N′G symmetric dimethylarginine (SDMA). The formation of methylated arginines is catalyzed by the protein arginine methyl transferases (PRMTs) family of methyltransferases. Currently, there are nine PRMTs annotated in the human genome. The majority of these enzymes are Type I enzymes (PRMT1, -2, -3, -4, -6, -8) that are capable of mono- and asymmetric dimethylation of arginine, with S-adenosylmethionine (SAM) as the methyl donor. PRMT-5, -7 and -9 are considered to be Type II enzymes that catalyze symmetric dimethylation of arginines. Each PRMT species harbors the characteristic motifs of seven beta strand methyltransferases (Katz et al., 2003), as well as additional “double E” and “THW” sequence motifs particular to the PRMT subfamily.

PRMT5 is as a general transcriptional repressor that functions with numerous transcription factors and repressor complexes, including BRG1 and hBRM, Blimp1, and Snail. This enzyme, once recruited to a promoter, symmetrically dimethylates H3R8 and H4R3. Importantly, the H4R3 site is a major target for PRMT1 methylation (ADMA) and is generally regarded as a transcriptional activating mark. Thus, both H4R3me2s (repressive; me2s indicates SDMA modification) and H4R3me2a (active; me2a indicates ADMA modification) marks are produced in vivo. The specificity of PRMT5 for H3R8 and H4R3 can be altered by its interaction with COPR5 and this could perhaps play an important role in determining PRMT5 corepressor status.

### Role of PRMTs in Cancer

Aberrant expression of PRMTs has been identified in human cancers, and PRMTs are considered to be therapeutic targets. Global analysis of histone modifications in prostate cancer has shown that the dimethylation of histone H4R3 is positively correlated with increasing grade, and these changes are predictive of clinical outcome.

PRMT5 levels have been shown to be elevated in a panel of lymphoid cancer cell lines as well as mantle cell lymphoma clinical samples. PRMT5 interacts with a number of substrates that are involved in a variety of cellular processes, including RNA processing, signal transduction, and transcriptional regulation. PRMT5 can directly modify histone H3 and H4, resulting in the repression of gene expression. PRMT5 overexpression can stimulate cell growth and induce transformation by directly repressing tumor suppressor genes. Pal et al., Mol. Cell. Biol. 2003, 7475; Pal et al. Mol. Cell. Biol. 2004, 9630; Wang et al. Mol. Cell. Biol. 2008, 6262; Chung et al. J Biol Chem 2013, 5534. In addition to its well-documented oncogenic functions in transcription and translation, the transcription factor MYC also safeguards proper pre-messenger-RNA splicing as an essential step in lymphomagenesis. Koh et al. Nature 2015, 523 7558; Hsu et al. Nature 2015 525, 384.

The discovery of cancer dependencies has the potential to inform therapeutic strategies and to identify putative drug targets. Integrating data from comprehensive genomic profiling of cancer cell lines and from functional characterization of cancer cell dependencies, it has been recently discovered that loss of the enzyme methylthioadenosine phosphorylase (MTAP) confers a selective dependence on protein arginine methyltransferase 5 (PRMT5) and its binding partner WDR77. MTAP is frequently lost due to its proximity to the commonly deleted tumor suppressor gene, CDKN2A. Cells harboring MTAP deletions possess increased intracellular concentrations of methylthioadenosine (MTA, the metabolite cleaved by MTAP). Furthermore, MTA specifically inhibits PRMT5 enzymatic activity. Administration of either MTA or a small-molecule PRMT5 inhibitor shows a preferential impairment of cell viability for MTAP-null cancer cell lines compared to isogenic MTAP-expressing counterparts. Together, these findings reveal PRMT5 as a potential vulnerability across multiple cancer lineages augmented by a common “passenger” genomic alteration.

### Role of PRMT5 in Hemoglobinopathies

The developmental switch in human globin gene subtype from fetal to adult that begins at birth heralds the onset of the hemoglobinopathies, b-thalassemia and sickle cell disease (SCD). The observation that increased adult globin gene expression (in the setting of hereditary persistence of fetal hemoglobin [HPFH] mutations) significantly ameliorates the clinical severity of thalassemia and SCD has prompted the search for therapeutic strategies to reverse gamma-globin gene silencing. Central to silencing of the gamma-genes is DNA methylation, which marks critical CpG dinucleotides flanking the gene transcriptional start site in adult bone marrow erythroid cells. It has been shown that these marks are established as a consequence of recruitment of the DNA methyltransferase, DNMT3A to the gamma-promoter by the protein arginine methyltransferase PRMT5. Zhao et al. Nat Struct Mol Biol. 2009 16, 304. PRMT5-mediated methylation of histone H4R3 recruits DNMT3A, coupling histone and DNA methylation in gene silencing.

PRMT5 induces the repressive histone mark, H4R3me2s, which serves as a template for direct binding of DNMT3A, and subsequent DNA methylation. Loss of PRMT5 binding or its enzymatic activity leads to demethylation of the CpG dinucleotides and gene activation. In addition to the H4R3me2s mark and DNA methylation, PRMT5 binding to the gamma-promoter, and its enzymatic activity are essential for assembly of a multiprotein complex on the gamma-promoter, which induces a range of coordinated repressive epigenetic marks. Disruption of this complex leads to reactivation of gamma gene expression. These studies provide the basis for developing PRMT5 inhibitors as targeted therapies for thalassemia and SCD.

### SUMMARY

The disclosure is directed to pharmaceutically acceptable salts of (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol, i.e., the compound of Formula I:

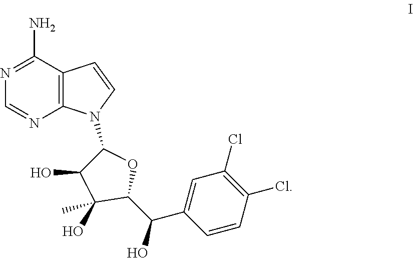 [SMILES: C[C@@]1(O)[C@@H]([C@H](O)c2ccc(Cl)c(Cl)c2)O[C@@H](n2ccc3c(N)ncnc32)[C@@H]1O]

The disclosure is also directed to maleate, hydrochloride, oxalate, phosphate, and bisulfate salts of Formula I.

Crystalline forms of such salts, as well as pharmaceutical compositions containing such salts and methods of use of such salts are also described.

The disclosure is also directed to crystalline forms of the compound of Formula I, as well as pharmaceutical compositions containing such forms and methods of use of such forms are also described.

### BRIEF DESCRIPTION OF THE DRAWINGS

FIG. 1 shows an XRPD of a maleate salt having Formula IA.

FIG. 2 shows an XRPD of a maleate salt having Formula IA.

FIG. 3 shows a DSC thermogram of a maleate salt having Formula IA.

FIG. 4 shows a TGA profile of a maleate salt having Formula IA.

FIG. 5 shows a TGA profile and DSC thermogram of a maleate salt having Formula IA.

FIG. 6 shows an XRPD of a hydrochloride salt having Formula IB.

FIG. 7 shows an XRPD of a hydrochloride salt having Formula IB.

FIG. 8 shows an XRPD of a hydrochloride salt having Formula IB.

FIG. 9 shows a DSC thermogram of a hydrochloride salt having Formula IB.

FIG. 10 shows a TGA profile of a hydrochloride salt having Formula IB.

FIG. 11 shows a TGA profile and DSC thermogram of a hydrochloride salt having Formula IB.

FIG. 12 shows an XRPD of an oxalate salt having Formula IC.

FIG. 13 shows an XRPD of a phosphate salt having Formula ID.

FIG. 14 shows an XRPD of a maleate salt having Formula IA.

FIG. 15 shows a DSC thermogram of a maleate salt having Formula IA.

FIG. 16 shows a TGA profile of a maleate salt having Formula IA.

FIG. 17 shows an XRPD of a hydrochloride salt having Formula IB.

FIG. 18 shows a DSC thermogram of a hydrochloride salt having Formula IB.

FIG. 19 shows a TGA profile of a hydrochloride salt having Formula IB.

FIG. 20 shows an XRPD of Formula IB, Form I.

FIG. 21 shows a DSC thermogram of Formula IB, Form I.

FIG. 22 shows a TGA profile of Formula IB, Form I.

FIG. 23 shows a DVS profile of Formula IB, Form I.

FIG. 24 shows a comparison of the XRPD of Formula IB, Form I, before (top) and after (bottom) DVS.

FIG. 25 shows the ¹H NMR (400 MHz; DMSO-d₆) of Formula IB, Form I.

FIG. 26 shows XRPD shows an XRPD of Formula IB, Form II.

FIG. 27 shows a DSC thermogram of Formula IB, Form II.

FIG. 28 shows a TGA profile of Formula IB, Form II.

FIG. 29 shows the ¹H NMR (400 MHz; MeOH-d₄) of Formula IB, Form II.

FIG. 30 shows a DVS profile of of Formula IB, Form II.

FIG. 31 shows a comparison of the XRPD of Formula IB, Form II, before (top) and after (bottom) DVS.

FIG. 32 shows an XRPD of Formula IB, Form III.

FIG. 33 shows a DSC thermogram of Formula IB, Form III.

FIG. 34 shows a TGA profile of Formula IB, Form III.

FIG. 35 shows the ¹H NMR (400 MHz; DMSO-d₆) of Formula IB, Form III.

FIG. 36 shows a DVS profile of Formula IB, Form III.

FIG. 37 shows a comparison of the XRPD of Formula IB, Form III, before (top) and after (bottom) DVS.

FIG. 38 shows an XRPD of Formula IB, Form IV.

FIG. 39 shows a DSC thermogram for Formula IB, Form IV.

FIG. 40 shows a TGA profile for Formula IB, Form IV.

FIG. 41 shows an ¹H NMR (400 MHz; DMSO-d₆) of Formula IB, Form IV.

FIG. 42 shows an XRPD of a crystalline form of Formula IB.

FIG. 43 shows a DSC thermogram of a crystalline form of Formula IB.

FIG. 44 shows a TGA profile of a crystalline form of Formula IB.

FIG. 45 shows an XRPD of a phosphate salt having Formula ID.

FIG. 46 shows a DSC thermogram of a phosphate salt having Formula ID.

FIG. 47 shows a TGA profile of a phosphate salt having Formula ID.

FIG. 48 shows an XRPD of a crystalline form of the compound having Formula I, Form I.

FIG. 49 shows a DSC thermogram of a crystalline form of the compound having Formula I, Form I.

FIG. 50 shows a TGA profile for Formula I, Form I.

FIG. 51 shows an ¹H NMR (400 MHz; MeOH-d₄) for Formula I, Form I.

FIG. 52 shows a DVS profile for Formula I, Form I.

FIG. 53 shows a comparison of the XRPD before (top) and after (bottom) DVS for Formula I, Form I.

FIG. 54 shows a XRPD of Formula I, Form II.

FIG. 55 shows a DSC thermogram for Formula I, Form II.

FIG. 56 shows an XRPD of Formula I, Form III.

FIG. 57 shows a DSC thermogram for Formula I, Form III.

FIG. 58 shows a XRPD of Formula I, Form II.

FIG. 59 shows a DSC thermogram for Formula I, Form II.

FIG. 60 shows a XRPD of Formula I, Form II.

FIG. 61 shows a DSC thermogram for Formula I, Form II.

FIG. 62 shows a XRPD of Formula I, Form II.

FIG. 63 shows a DSC thermogram for Formula I, Form II.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, phosphate, sulfate, bisulfate, and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace pharmaceutically acceptable salts of compounds of Formula I as described herein, as well as their subgenera, which expression includes the stereoisomers (e.g., enantiomers, diastereomers) and constitutional isomers (e.g., tautomers) where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

In some aspects, the disclosure is directed to pharmaceutically acceptable salts of the compound of Formula I:

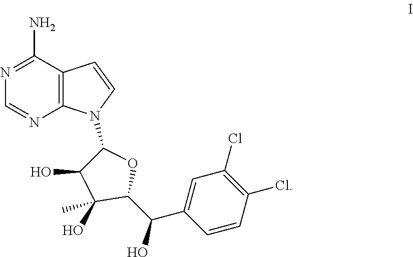 [SMILES: C[C@@]1(O)[C@@H]([C@H](O)c2ccc(Cl)c(Cl)c2)O[C@@H](n2ccc3c(N)ncnc32)[C@@H]1O]

In some embodiments, the pharmaceutically acceptable salt of the compound of Formula I is the maleate salt, which has the formula IA:

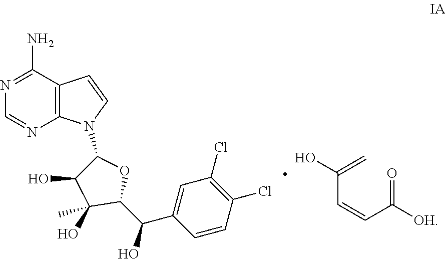

In some embodiments, the pharmaceutically acceptable salt of the compound of Formula I is the hydrochloride salt, which has the formula IB:

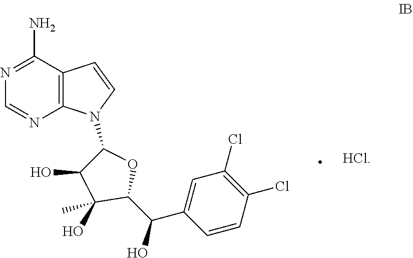 [SMILES: C[C@@]1(O)[C@@H]([C@H](O)c2ccc(Cl)c(Cl)c2)O[C@@H](n2ccc3c(N)ncnc32)[C@@H]1O ; "HCl."]

In some embodiments, the pharmaceutically acceptable salt of the compound of Formula I is the oxalate salt, which has the formula IC:

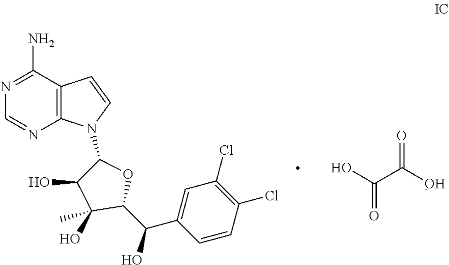 [SMILES: C[C@@]1(O)[C@@H]([C@H](O)c2ccc(Cl)c(Cl)c2)O[C@@H](n2ccc3c(N)ncnc32)[C@@H]1O ; O=C(O)C(=O)O]

In some embodiments, the pharmaceutically acceptable salt of the compound of Formula I is the phosphate salt, which has the formula ID:

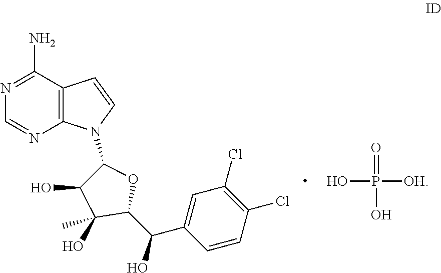 [SMILES: C[C@@]1(O)[C@@H]([C@H](O)c2ccc(Cl)c(Cl)c2)O[C@@H](n2ccc3c(N)ncnc32)[C@@H]1O ; O=P(O)(O)O]

In some embodiments, the pharmaceutically acceptable salt of the compound of Formula I is the bisulfate salt, which has the formula IE:

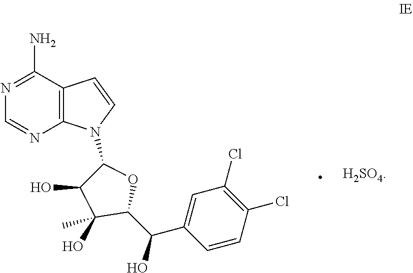 [SMILES: C[C@@]1(O)[C@@H]([C@H](O)c2ccc(Cl)c(Cl)c2)O[C@@H](n2ccc3c(N)ncnc32)[C@@H]1O ; "H2SO4."]

In some aspects, the disclosure is directed to crystalline forms of pharmaceutically acceptable salts of Formula I.

In some embodiments, the disclosure is directed to crystalline forms of the salts of Formula IA, IB, IC, ID, or IE.

In other aspects, the disclosure is directed to crystalline forms of the compound of Formula I.

The crystalline forms of the salts of Formula IA, IB, IC, ID, or IE, and the crystalline forms of Formula I, according to the present disclosure may have advantageous properties, including, one or more of chemical or polymorphic purity, flowability, solubility, dissolution rate, bioavailability, morphology, or crystal habit, stability—e.g., chemical stability, thermal stability, and mechanical stability with respect to polymorphic conversion, storage stability; hygroscopicity, low content of residual solvents and advantageous processing and handling characteristics such as compressibility, or bulk density.

A crystal form may be referred to herein as being characterized by graphical data “as shown in” a Figure. Such data include, for example, powder X-ray diffractograms (XRPD), Differential Scanning Calorimetry (DSC) thermograms, or thermogravimetric analysis (TGA) profiles. As is known in the art, the graphical data potentially provides additional technical information to further define the respective solid state form which can not necessarily be described by reference to numerical values or peak positions alone. Thus, the term “substantially as shown in” when referring to graphical data in a Figure herein means a pattern that is not necessarily identical to those depicted herein, but that falls within the limits of experimental error or deviations, when considered by one of ordinary skill in the art. The skilled person would readily be able to compare the graphical data in the Figures herein with graphical data generated for an unknown crystal form and confirm whether the two sets of graphical data are characterizing the same crystal form or two different crystal forms.

A solid, crystalline form may be referred to herein as “polymorphically pure” or as “substantially free of any other form.” As used herein in this context, the expression “substantially free of any other forms” will be understood to mean that the solid form contains about 20% or less, about 10% or less, about 5% or less, about 2% or less, about 1% or less, or 0% of any other forms of the subject compound as measured, for example, by XRPD. For example, a solid form of Formula IA described herein as substantially free of any other solid forms would be understood to contain greater than about 80% (w/w), greater than about 90% (w/w), greater than about 95% (w/w), greater than about 98% (w/w), greater than about 99% (w/w), or about 100% of the subject solid form of Formula IA Accordingly, in some embodiments of the disclosure, the described solid forms of Formula IA may contain from about 1% to about 20% (w/w), from about 5% to about 20% (w/w), or from about 5% to about 10% (w/w) of one or more other solid forms of Formula IA.

As used herein, unless stated otherwise, XRPD peaks reported herein are measured using CuK_{α} radiation, λ=1.54 Å.

The modifier “about” should be considered as disclosing the range defined by the absolute values of the two endpoints. For example, the expression “from about 2 to about 4” also discloses the range “from 2 to 4.” When used to modify a single number, the term “about” refers to plus or minus 10% of the indicated number and includes the indicated number. For example, “about 10%” indicates a range of 9% to 11%, and “about 1” means from 0.9-1.1.

### Formula IA (Formula I Maleate Salt)

In some aspects, the disclosure is directed to a crystalline form of the maleate salt of Formula I, i.e., Formula IA. In some embodiments, the crystalline form of Formula IA is substantially free of any other solid form of Formula IA.

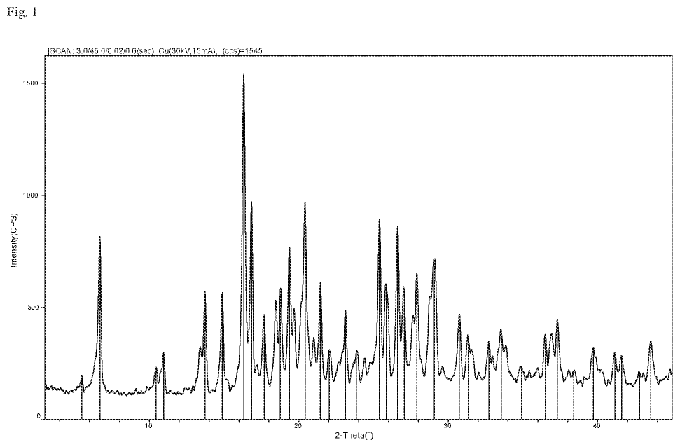

In some embodiments, the crystalline form of Formula IA exhibits an XRPD substantially as shown in FIG. 1. The XRPD of crystalline form of Formula IA shown in FIG. 1 comprises reflection angles (degrees 2-theta±0.2 degrees 2-theta), line spacings (d values), and relative intensities as shown in Table 1:

TABLE 1
XRPD Data for crystalline form
of Formula IA shown in Fig. 1

| Angle | | |
|---|---|---|
| (degrees 2- | | |
| theta ± 0.2 | | |
| degrees 2- | d Value | Relative |
| theta) | (Å) | Intensity |
| 5.479 | 16.1157 | 5 |
| 6.68 | 13.2205 | 50.3 |
| 10.441 | 8.4659 | 8.5 |
| 10.959 | 8.0664 | 13.4 |
| 13.72 | 6.4489 | 32.4 |
| 14.879 | 5.9489 | 31.8 |
| 16.319 | 5.4271 | 100 |
| 16.839 | 5.2606 | 59.6 |
| 17.679 | 5.0126 | 20.9 |
| 18.762 | 4.7256 | 26.1 |
| 19.379 | 4.5765 | 39.8 |
| 20.419 | 4.3457 | 53.7 |
| 21.441 | 4.1408 | 28.4 |
| 22.04 | 4.0296 | 9.1 |
| 23.12 | 3.8438 | 22.5 |
| 23.862 | 3.726 | 9.2 |
| 25.4 | 3.5037 | 51 |
| 25.859 | 3.4426 | 27.4 |
| 26.619 | 3.3459 | 46.7 |
| 27.057 | 3.2928 | 27 |
| 27.9 | 3.1952 | 31.8 |
| 29.08 | 3.0681 | 38.2 |
| 30.741 | 2.9061 | 21.1 |
| 31.358 | 2.8503 | 12.6 |
| 32.738 | 2.7332 | 11 |
| 33.539 | 2.6697 | 16.3 |
| 34.917 | 2.5675 | 4.3 |
| 36.499 | 2.4597 | 14.8 |
| 37.3 | 2.4087 | 19.4 |
| 38.401 | 2.3422 | 3.8 |
| 39.72 | 2.2674 | 12 |
| 41.16 | 2.1913 | 10.5 |
| 41.618 | 2.1683 | 9.6 |
| 42.817 | 2.1103 | 3.8 |
| 43.56 | 2.076 | 13.8 |

In some embodiments of the present disclosure, the crystalline form of Formula IA is characterized by an XRPD pattern comprising a peak at one of the angles listed in Table 1. In other aspects, the crystalline form of Formula IA is characterized by an XRPD pattern comprising more than one peak at one of the angles listed in Table 1 above. In other aspects, the crystalline form of Formula IA is characterized by an XRPD pattern comprising two peaks selected from the angles listed in Table 1 above. In other aspects, the crystalline form of Formula IA is characterized by an XRPD pattern comprising three peaks selected from the angles listed in Table 1 above. In other aspects, the crystalline form of Formula IA is characterized by an XRPD pattern comprising four peaks selected from the angles listed in Table 1 above. In other aspects, the crystalline form of Formula IA is characterized by an XRPD pattern comprising five peaks selected from the angles listed in Table 1 above. In other aspects, the crystalline form of Formula IA is characterized by an XRPD pattern comprising six peaks selected from the angles listed in Table 1 above. In other aspects, the crystalline form of Formula IA is characterized by an XRPD pattern comprising seven peaks selected from the angles listed in Table 1 above. In other aspects, the crystalline form of Formula IA is characterized by an XRPD pattern comprising eight peaks selected from the angles listed in Table 1 above. In other aspects, the crystalline form of Formula IA is characterized by an XRPD pattern comprising nine peaks selected from the angles listed in Table 1 above. In other aspects, the crystalline form of Formula IA is characterized by an XRPD pattern comprising ten peaks selected from the angles listed in Table 1 above. In other aspects, the crystalline form of Formula IA is characterized by an XRPD pattern comprising more than ten peaks selected from the angles listed in Table 1 above.

In some embodiments, the crystalline form of Formula IA is characterized by an XRPD pattern comprising a peak at 16.3 degrees±0.2 degrees 2-theta. In other embodiments, the crystalline form of Formula IA is characterized by an XRPD pattern comprising peaks at 6.7, 11.0, and 16.3 degrees±0.2 degrees 2-theta. In other embodiments, the crystalline form of Formula IA is characterized by an XRPD pattern comprising peaks at 6.7, 16.3, 20.4, and 30.7 degrees±0.2 degree 2-theta. In other embodiments, the crystalline form of Formula IA is characterized by an XRPD pattern comprising peaks at 6.7, 14.9, 16.3, and 20.4 degrees±0.2 degree 2-theta. In other embodiments, the crystalline form of Formula IA is characterized by an XRPD pattern comprising peaks at 6.7, 11.0, 14.9, 16.3, 16.8, 20.4, 25.4 degrees±0.2 degree 2-theta. In yet other embodiments, the crystalline form of Formula IA is characterized by an XRPD pattern comprising peaks at 6.7, 16.3, 20.4, 25.4, and 30.7 degrees±0.2 degree 2-theta. In yet other embodiments, the crystalline form of Formula IA is characterized by an XRPD pattern comprising peaks at 6.7, 11.0, 14.9, 16.3, 16.8, 20.4, 25.4, 25.9, 27.9, 29.1, and 30.7 degrees±0.2 degree 2-theta.

In some embodiments of the present disclosure, the crystalline form of Formula IA is characterized by an XRPD pattern comprising peaks at three or more of 6.7, 11.0, 14.9, 16.3, 16.8, 20.4, 25.4, 25.9, 27.9, 29.1, and 30.7 degrees±0.2 degrees 2-theta. In some embodiments of the present disclosure, the crystalline form of Formula IA is characterized by an XRPD pattern comprising peaks at four or more of 6.7, 11.0, 14.9, 16.3, 16.8, 20.4, 25.4, 25.9, 27.9, 29.1, and 30.7 degrees±0.2 degrees 2-theta. In some embodiments of the present disclosure, the crystalline form of Formula IA is characterized by an XRPD pattern comprising peaks at five or more of 6.7, 11.0, 14.9, 16.3, 16.8, 20.4, 25.4, 25.9, 27.9, 29.1, and 30.7 degrees±0.2 degrees 2-theta. In some embodiments of the present disclosure, the crystalline form of Formula IA is characterized by an XRPD pattern comprising peaks at six or more of 6.7, 11.0, 14.9, 16.3, 16.8, 20.4, 25.4, 25.9, 27.9, 29.1, and 30.7 degrees±0.2 degrees 2-theta. In some embodiments of the present disclosure, the crystalline form of Formula IA is characterized by an XRPD pattern comprising peaks at seven or more of 6.7, 11.0, 14.9, 16.3, 16.8, 20.4, 25.4, 25.9, 27.9, 29.1, and 30.7 degrees±0.2 degrees 2-theta.

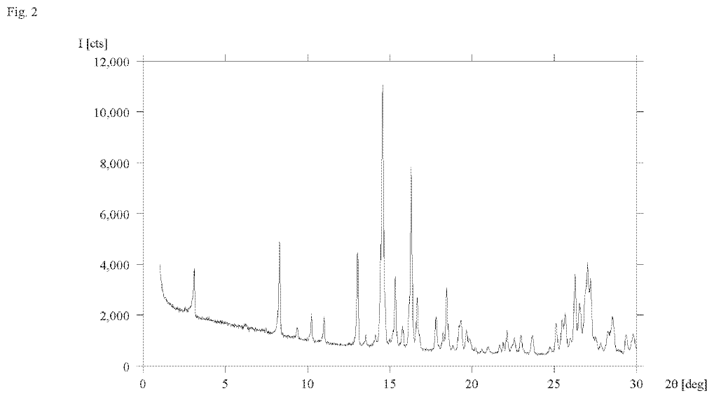

In some embodiments, the crystalline form of Formula IA exhibits an XRPD substantially as shown in FIG. 2. The XRPD of crystalline form of Formula IA shown in FIG. 2 comprises reflection angles (degrees 2-theta±0.2 degrees 2-theta), line spacings (d values), and relative intensities as shown in Table 2:

TABLE 2
XRPD Data for crystalline form
of Formula IA shown in Fig. 2

| Angle | | |
|---|---|---|
| (degrees 2- | | |
| theta ± 0.2 | | |
| degrees 2- | d Value | Relative |
| theta) | (Å) | Intensity |
| 3.10 | 28.439 | 34 |
| 8.29 | 10.656 | 45 |
| 9.36 | 9.443 | 13 |
| 10.23 | 8.639 | 18 |
| 11.00 | 8.038 | 18 |
| 13.03 | 6.789 | 40 |
| 13.53 | 6.538 | 11 |
| 14.12 | 6.266 | 11 |
| 14.56 | 6.080 | 100 |
| 15.33 | 5.777 | 32 |
| 15.77 | 5.615 | 14 |
| 16.30 | 5.434 | 70 |
| 16.66 | 5.317 | 24 |
| 17.81 | 4.977 | 18 |
| 18.22 | 4.865 | 12 |
| 18.45 | 4.805 | 28 |
| 18.83 | 4.710 | 7 |
| 19.32 | 4.592 | 16 |
| 19.66 | 4.511 | 13 |
| 19.84 | 4.471 | 10 |
| 20.19 | 4.394 | 7 |
| 20.60 | 4.308 | 6 |
| 20.96 | 4.234 | 7 |
| 21.67 | 4.098 | 8 |
| 21.90 | 4.055 | 8 |
| 22.12 | 4.015 | 13 |
| 22.57 | 3.936 | 10 |
| 22.96 | 3.870 | 11 |
| 23.66 | 3.757 | 11 |
| 24.73 | 3.597 | 7 |
| 25.11 | 3.543 | 15 |
| 25.48 | 3.493 | 17 |
| 25.66 | 3.469 | 19 |
| 25.97 | 3.428 | 10 |
| 26.26 | 3.392 | 33 |
| 26.53 | 3.356 | 23 |
| 27.01 | 3.298 | 36 |
| 27.19 | 3.277 | 31 |
| 27.52 | 3.239 | 10 |
| 27.82 | 3.204 | 8 |
| 28.27 | 3.155 | 12 |
| 28.55 | 3.124 | 17 |
| 29.36 | 3.040 | 11 |
| 29.79 | 2.997 | 11 |

In some embodiments of the present disclosure, the crystalline form of Formula IA is characterized by an XRPD pattern comprising a peak at one of the angles listed in Table 2. In other aspects, the crystalline form of Formula IA is characterized by an XRPD pattern comprising more than one peak at one of the angles listed in Table 2 above. In other aspects, the crystalline form of Formula IA is characterized by an XRPD pattern comprising two peaks selected from the angles listed in Table 2 above. In other aspects, the crystalline form of Formula IA is characterized by an XRPD pattern comprising three peaks selected from the angles listed in Table 2 above. In other aspects, the crystalline form of Formula IA is characterized by an XRPD pattern comprising four peaks selected from the angles listed in Table 2 above. In other aspects, the crystalline form of Formula IA is characterized by an XRPD pattern comprising five peaks selected from the angles listed in Table 2 above. In other aspects, the crystalline form of Formula IA is characterized by an XRPD pattern comprising six peaks selected from the angles listed in Table 2 above. In other aspects, the crystalline form of Formula IA is characterized by an XRPD pattern comprising seven peaks selected from the angles listed in Table 2 above. In other aspects, the crystalline form of Formula IA is characterized by an XRPD pattern comprising eight peaks selected from the angles listed in Table 2 above. In other aspects, the crystalline form of Formula IA is characterized by an XRPD pattern comprising nine peaks selected from the angles listed in Table 2 above. In other aspects, the crystalline form of Formula IA is characterized by an XRPD pattern comprising ten peaks selected from the angles listed in Table 2 above. In other aspects, the crystalline form of Formula IA is characterized by an XRPD pattern comprising more than ten peaks selected from the angles listed in Table 2 above.

In some embodiments, the crystalline form of Formula IA is characterized by an XRPD pattern comprising a peak at 14.6 degrees±0.2 degrees 2-theta. In other embodiments, the crystalline form of Formula IA is characterized by an XRPD pattern comprising peaks at 13.0, 14.6, and 16.3 degrees±0.2 degrees 2-theta. In other embodiments, the crystalline form of Formula IA is characterized by an XRPD pattern comprising peaks at 8.3, 13.0, 14.6, 16.3, 26.3, and 27.0 degrees±0.2 degree 2-theta. In other embodiments, the crystalline form of Formula IA is characterized by an XRPD pattern comprising peaks at 8.3, 13.0, 14.6, 15.3, 16.3, 16.7, 27.0, and 27.2 degrees±0.2 degree 2-theta. In other embodiments, the crystalline form of Formula IA is characterized by an XRPD pattern comprising peaks at 3.1, 8.3, 13.0, 14.6, 15.3, and 16.3 degrees±0.2 degree 2-theta. In yet other embodiments, the crystalline form of Formula IA is characterized by an XRPD pattern comprising peaks at 14.6, 15.3, 16.3, 16.7, 18.4, 26.3, 27.0, and 27.2 degrees±0.2 degree 2-theta. In yet other embodiments, the crystalline form of Formula IA is characterized by an XRPD pattern comprising peaks at 3.1, 8.3, 13.0, 14.6, 15.3, 16.3, 16.7, 18.4, 26.3, 26.5, 27.0, and 27.2 degrees±0.2 degree 2-theta.

In some embodiments of the present disclosure, the crystalline form of Formula IA is characterized by an XRPD pattern comprising peaks at three or more of 3.1, 8.3, 13.0, 14.6, 15.3, 16.3, 16.7, 18.4, 26.3, 26.5, 27.0, and 27.2 degrees±0.2 degrees 2-theta. In some embodiments of the present disclosure, the crystalline form of Formula IA is characterized by an XRPD pattern comprising peaks at four or more of 3.1, 8.3, 13.0, 14.6, 15.3, 16.3, 16.7, 18.4, 26.3, 26.5, 27.0, and 27.2 degrees±0.2 degrees 2-theta. In some embodiments of the present disclosure, the crystalline form of Formula IA is characterized by an XRPD pattern comprising peaks at five or more of 3.1, 8.3, 13.0, 14.6, 15.3, 16.3, 16.7, 18.4, 26.3, 26.5, 27.0, and 27.2 degrees±0.2 degrees 2-theta. In some embodiments of the present disclosure, the crystalline form of Formula IA is characterized by an XRPD pattern comprising peaks at six or more of 3.1, 8.3, 13.0, 14.6, 15.3, 16.3, 16.7, 18.4, 26.3, 26.5, 27.0, and 27.2 degrees±0.2 degrees 2-theta. In some embodiments of the present disclosure, the crystalline form of Formula IA is characterized by an XRPD pattern comprising peaks at seven or more of 3.1, 8.3, 13.0, 14.6, 15.3, 16.3, 16.7, 18.4, 26.3, 26.5, 27.0, and 27.2 degrees±0.2 degrees 2-theta.

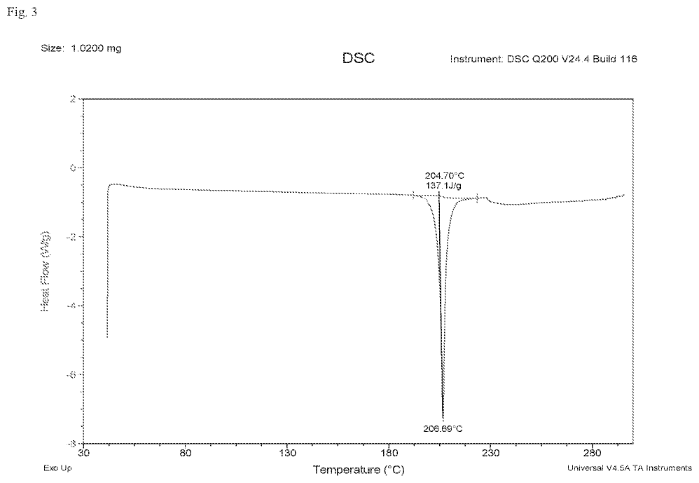

In some embodiments, the crystalline form of Formula IA can be characterized by a DSC thermogram substantially as shown in FIG. 3. As FIG. 3 shows, the crystalline form of Formula IA produced an endothermic peak at 206.69° C., with a peak onset temperature of 204.70° C., and an enthalpy of melting of 137.1 J/g, when heated at a rate of 10° C./min. In some embodiments of the present disclosure, the crystalline form of Formula IA is characterized by a DSC thermogram comprising an endothermic peak at about 207° C. In other embodiments of the present disclosure, the crystalline form of Formula IA is characterized by a DSC enthalpy of melting of about 137 J/g.

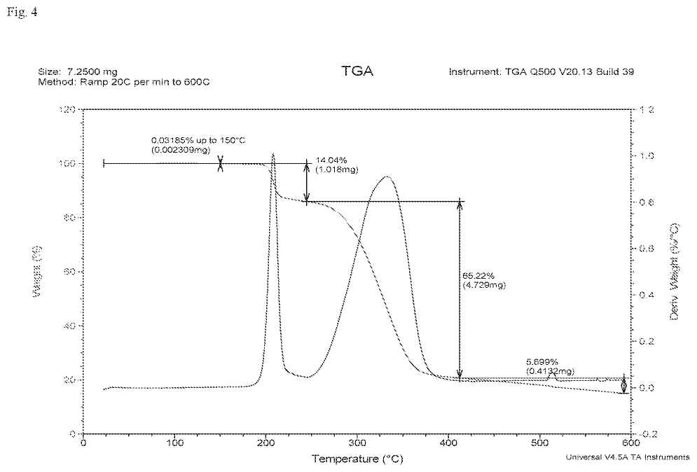

In some embodiments, the crystalline form of Formula IA can be characterized by a TGA profile substantially as shown in FIG. 4 when heated at a rate of 20° C./min. As FIG. 4 shows, the crystalline form of Formula IA lost about 0.03% of its weight upon heating to about 150° C.

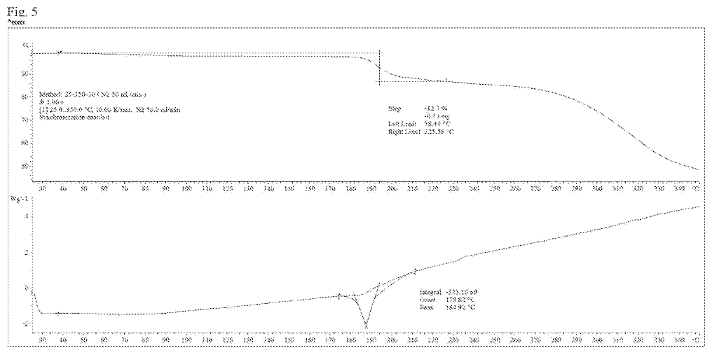

In some embodiments, the crystalline form of Formula IA can be characterized by a DSC thermogram and TGA profile substantially as shown in FIG. 5. As FIG. 5 shows, the crystalline form of Formula IA produced an endothermic peak at 184.92° C., with a peak onset temperature of 179.82° C. when heated at a rate of 10 K/min.

In some embodiments of the present disclosure, the crystalline form of Formula IA is characterized by a DSC thermogram comprising an endothermic peak at about 185° C. when heated at a rate of 10 K/min. As FIG. 5 shows, the crystalline form of Formula IA lost about 12.3% of its weight upon heating to about 210° C.

In some embodiments of the present disclosure, the crystalline form of Formula IA is characterized by an XRPD pattern comprising peaks at 6.7, 14.9, 16.3, and 20.4 degrees±0.2 degrees 2-theta, and a DSC thermogram comprising an endothermic peak at about 207° C. when heated at a rate of 10° C./min.

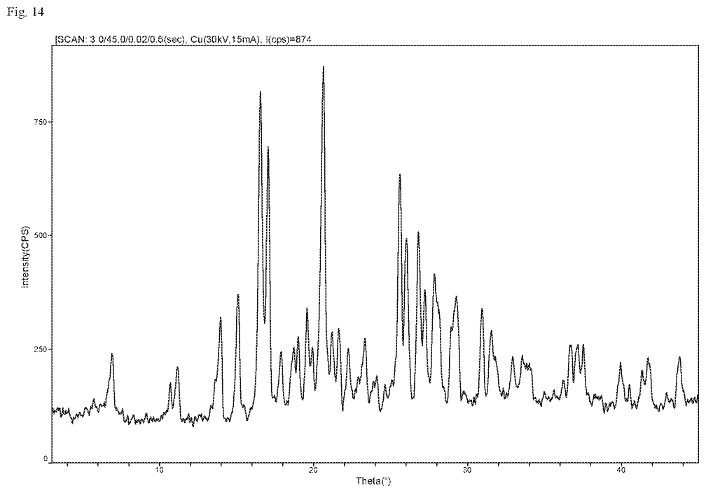

In some embodiments, the crystalline form of Formula IA exhibits an XRPD substantially as shown in FIG. 14.

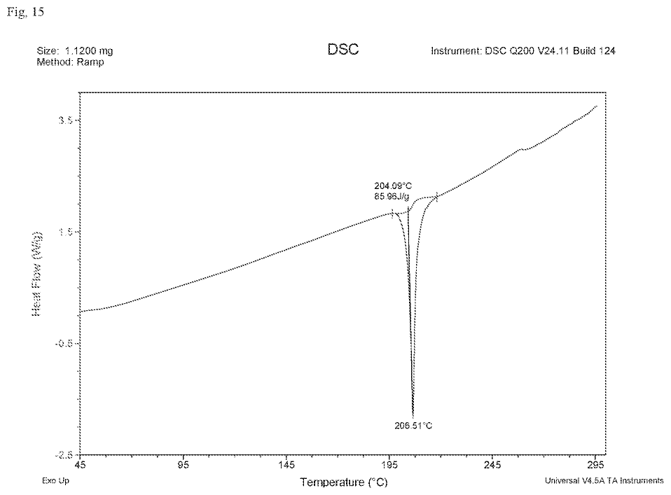

In some embodiments, the crystalline form of Formula IA exhibits a DSC thermogram substantially as shown in FIG. 15.

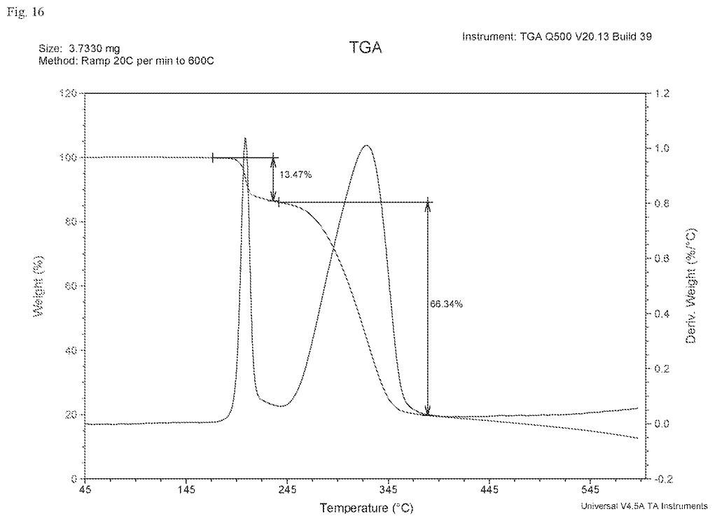

In some embodiments, the crystalline form of Formula IA exhibits a TGA substantially as shown in FIG. 16.

### Formula IB (Formula I HCl Salt)

In some aspects, the disclosure is directed to a crystalline form of the hydrochloride salt, i.e., Formula IB. In some embodiments, the crystalline form of Formula IB is substantially free of any other solid form of Formula IB.

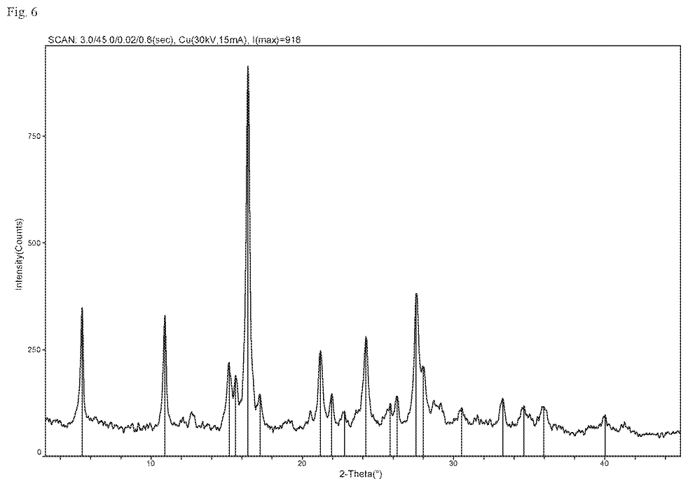

In some embodiments, a crystalline form of Formula IB exhibits an XRPD substantially as shown in FIG. 6. The XRPD of the crystalline form of Formula IB shown in FIG. 6 comprises reflection angles (degrees 2-theta±0.2 degrees 2-theta), line spacings (d values), and relative intensities as shown in Table 3:

TABLE 3
XRPD Data for crystalline form
of Formula IB shown in Fig. 6

| Angle | | |
|---|---|---|
| (degrees 2- | | |
| theta ± 0.2 | | |
| degrees 2- | d Value | Relative |
| theta) | (Å) | Intensity |
| 5.439 | 16.2337 | 33.7 |
| 10.901 | 8.1096 | 32.7 |
| 15.159 | 5.8399 | 19.7 |
| 15.58 | 5.6829 | 9.3 |
| 16.4 | 5.4005 | 100 |
| 17.2 | 5.1512 | 5.1 |
| 21.2 | 4.1874 | 21.1 |
| 21.941 | 4.0477 | 7.5 |
| 22.798 | 3.8974 | 4 |
| 24.2 | 3.6746 | 26 |
| 25.802 | 3.4501 | 5.8 |
| 26.26 | 3.3909 | 7.7 |
| 27.521 | 3.2384 | 36.3 |
| 28 | 3.184 | 13.8 |
| 30.521 | 2.9265 | 4.6 |
| 33.259 | 2.6916 | 8 |
| 34.642 | 2.5873 | 5.7 |
| 35.96 | 2.4954 | 5.8 |
| 40.019 | 2.2511 | 4.1 |

In some embodiments of the present disclosure, the crystalline form of Formula IB is characterized by an XRPD pattern comprising a peak at one of the angles listed in Table 3. In other aspects, the crystalline form of Formula IB is characterized by an XRPD pattern comprising more than one peak at one of the angles listed in Table 3 above. In other aspects, the crystalline form of Formula IB is characterized by an XRPD pattern comprising two peaks selected from the angles listed in Table 3 above. In other aspects, the crystalline form of Formula IB is characterized by an XRPD pattern comprising three peaks selected from the angles listed in Table 3 above. In other aspects, the crystalline form of Formula IB is characterized by an XRPD pattern comprising four peaks selected from the angles listed in Table 3 above. In other aspects, the crystalline form of Formula IB is characterized by an XRPD pattern comprising five peaks selected from the angles listed in Table 3 above. In other aspects, the crystalline form of Formula IB is characterized by an XRPD pattern comprising six peaks selected from the angles listed in Table 3 above. In other aspects, the crystalline form of Formula IB is characterized by an XRPD pattern comprising seven peaks selected from the angles listed in Table 3 above. In other aspects, the crystalline form of Formula IB is characterized by an XRPD pattern comprising eight peaks selected from the angles listed in Table 3 above. In other aspects, the crystalline form of Formula IB is characterized by an XRPD pattern comprising nine peaks selected from the angles listed in Table 3 above. In other aspects, the crystalline form of Formula IB is characterized by an XRPD pattern comprising ten peaks selected from the angles listed in Table 3 above. In other aspects, the crystalline form of Formula IB is characterized by an XRPD pattern comprising more than ten peaks selected from the angles listed in Table 3 above.

In some embodiments, the crystalline form of Formula IB is characterized by an XRPD pattern comprising a peak at 5.4 degrees±0.2 degrees 2-theta. In other embodiments, the crystalline form of Formula IB is characterized by an XRPD pattern comprising peaks at 5.4, 10.9, and 16.4 degrees±0.2 degrees 2-theta. In other embodiments, the crystalline form of Formula IB is characterized by an XRPD pattern comprising peaks at 5.4, 10.9, 21.2, and 24.2 degrees±0.2 degree 2-theta. In yet other embodiments, the crystalline form of Formula IB is characterized by an XRPD pattern comprising peaks at 5.4, 10.9, 16.4, 21.2, and 24.2 degrees±0.2 degree 2-theta. In yet other embodiments, the crystalline form of Formula IB is characterized by an XRPD pattern comprising peaks at 5.4, 10.9, 16.4, 21.2, 24.2, and 27.5 degrees±0.2 degree 2-theta.

In some embodiments of the present disclosure, the crystalline form of Formula IB is characterized by an XRPD pattern comprising peaks at three or more of 5.4, 10.9, 16.4, 21.2, 24.2, and 27.5 degrees±0.2 degrees 2-theta. In some embodiments of the present disclosure, the crystalline form of Formula IB is characterized by an XRPD pattern comprising peaks at four or more of 5.4, 10.9, 16.4, 21.2, 24.2, and 27.5 degrees±0.2 degrees 2-theta. In some embodiments of the present disclosure, the crystalline form of Formula IB is characterized by an XRPD pattern comprising peaks at five or more of 5.4, 10.9, 16.4, 21.2, 24.2, and 27.5 degrees±0.2 degrees 2-theta.

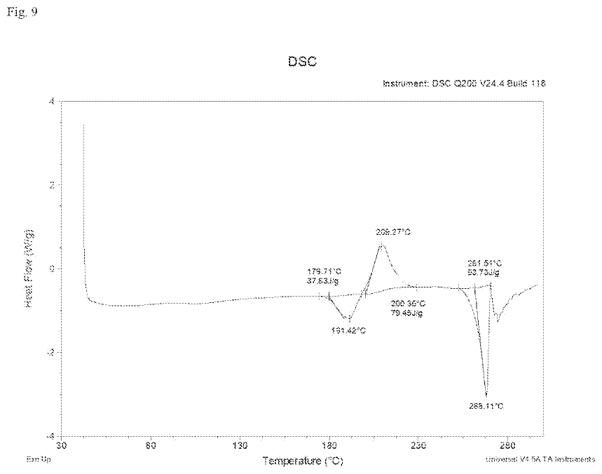

In some embodiments, the crystalline form of Formula IB can be characterized by a DSC thermogram substantially as shown in FIG. 9. As FIG. 9 shows, the crystalline form of Formula IB produced an endothermic peak at 191.42° C. (179.71° C. onset; 37.63 J/g), followed by an exothermic peak at 209.27° C. (200.36° C. onset; 79.45 J/g), followed by another endothermic peak at 268.11° C. (261.51° C. onset; 93.73 J/g), when heated at 10° C./min. In some embodiments of the present disclosure, the crystalline form of Formula IB is characterized by a DSC thermogram comprising an endothermic peak at about 191° C. when heated at a rate of 10° C./min. In other embodiments of the present disclosure, the crystalline form of Formula IB is characterized by a DSC thermogram comprising an endothermic peak at about 268° C. when heated at a rate of 10° C./min.

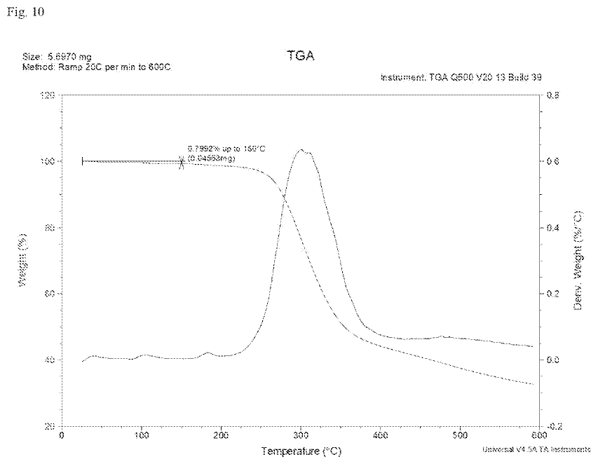

In some embodiments, the crystalline form of Formula IB can be characterized by a TGA profile substantially as shown in FIG. 10 when heated at a rate of 20° C./min. As FIG. 10 shows, the crystalline form of Formula IB lost about 0.8% of its weight upon heating to about 150° C.

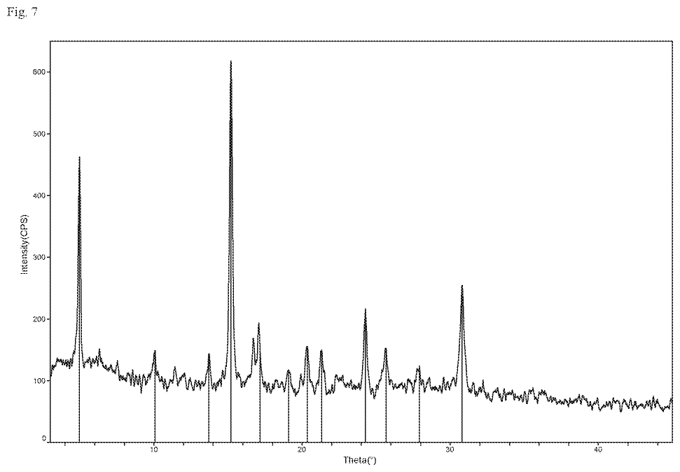

In some embodiments, a crystalline form of Formula IB exhibits an XRPD substantially as shown in FIG. 7. The XRPD of the crystalline form of Formula IB shown in FIG. 7 comprises reflection angles (degrees 2-theta±0.2 degrees 2-theta), line spacings (d values), and relative intensities as shown in Table 4:

TABLE 4
XRPD Data for crystalline form
of Formula IB shown in Fig. 7

| Angle | | |
|---|---|---|
| (degrees 2- | | |
| theta ± 0.2 | | |
| degrees 2- | d Value | Relative |
| theta) | (Å) | Intensity |
| 4.979 | 17.732 | 64.3 |
| 10.06 | 8.7856 | 12 |
| 13.72 | 6.4491 | 11.1 |
| 15.2 | 5.8241 | 100 |
| 17.153 | 5.165 | 12.4 |
| 19.099 | 4.643 | 7.3 |
| 20.359 | 4.3585 | 14.5 |
| 21.319 | 4.1642 | 13.3 |
| 24.28 | 3.6628 | 25.6 |
| 25.659 | 3.4689 | 14.5 |
| 27.919 | 3.1931 | 7.9 |
| 30.8 | 2.9006 | 33.1 |

In some embodiments of the present disclosure, the crystalline form of Formula IB is characterized by an XRPD pattern comprising a peak at one of the angles listed in Table 4. In other aspects, the crystalline form of Formula IB is characterized by an XRPD pattern comprising more than one peak at one of the angles listed in Table 4 above. In other aspects, the crystalline form of Formula IB is characterized by an XRPD pattern comprising two peaks selected from the angles listed in Table 4 above. In other aspects, the crystalline form of Formula IB is characterized by an XRPD pattern comprising three peaks selected from the angles listed in Table 4 above. In other aspects, the crystalline form of Formula IB is characterized by an XRPD pattern comprising four peaks selected from the angles listed in Table 4 above. In other aspects, the crystalline form of Formula IB is characterized by an XRPD pattern comprising five peaks selected from the angles listed in Table 4 above. In other aspects, the crystalline form of Formula IB is characterized by an XRPD pattern comprising six peaks selected from the angles listed in Table 4 above. In other aspects, the crystalline form of Formula IB is characterized by an XRPD pattern comprising seven peaks selected from the angles listed in Table 4 above. In other aspects, the crystalline form of Formula IB is characterized by an XRPD pattern comprising eight peaks selected from the angles listed in Table 4 above. In other aspects, the crystalline form of Formula IB is characterized by an XRPD pattern comprising nine peaks selected from the angles listed in Table 4 above. In other aspects, the crystalline form of Formula IB is characterized by an XRPD pattern comprising ten peaks selected from the angles listed in Table 4 above. In other aspects, the crystalline form of Formula IB is characterized by an XRPD pattern comprising more than ten peaks selected from the angles listed in Table 4 above.

In other embodiments, the crystalline form of Formula IB is characterized by an XRPD pattern comprising a peak at 5.0 degrees±0.2 degrees 2-theta. In other embodiments, the crystalline form of Formula IB is characterized by an XRPD pattern comprising peaks at 5.0, 15.2, and 24.3 degrees±0.2 degrees 2-theta. In other embodiments, the crystalline form of Formula IB is characterized by an XRPD pattern comprising peaks at 5.0, 15.2, 24.3, and 30.8 degrees±0.2 degree 2-theta. In yet other embodiments, the crystalline form of Formula IB is characterized by an XRPD pattern comprising peaks at 5.0, 10.1, 13.7, 15.2, 17.1, 24.3, and 30.8 degrees±0.2 degree 2-theta. In yet other embodiments, the crystalline form of Formula IB is characterized by an XRPD pattern comprising peaks at 17.1, 24.3, and 30.8 degrees±0.2 degree 2-theta.

In some embodiments of the present disclosure, the crystalline form of Formula IB is characterized by an XRPD pattern comprising peaks at three or more of 5.0, 10.1, 13.7, 15.2, 17.1, 24.3, and 30.8 degrees±0.2 degrees 2-theta. In some embodiments of the present disclosure, the crystalline form of Formula IB is characterized by an XRPD pattern comprising peaks at four or more of 5.0, 10.1, 13.7, 15.2, 17.1, 24.3, and 30.8 degrees±0.2 degrees 2-theta. In some embodiments of the present disclosure, the crystalline form of Formula IB is characterized by an XRPD pattern comprising peaks at five or more of 5.0, 10.1, 13.7, 15.2, 17.1, 24.3, and 30.8 degrees±0.2 degrees 2-theta.

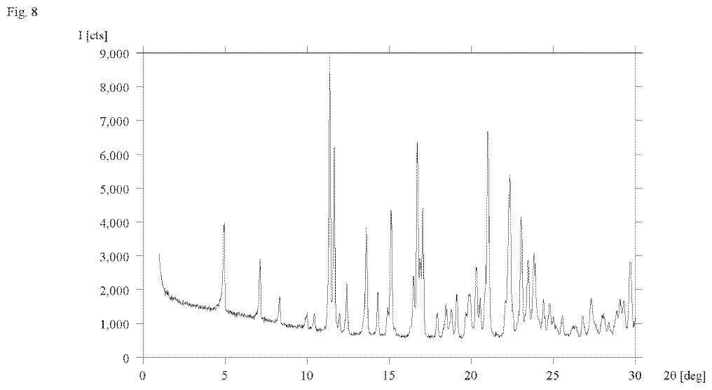

In some embodiments, a crystalline form of Formula IB exhibits an XRPD substantially as shown in FIG. 8. The XRPD of crystalline form of Formula IB shown in FIG. 8 comprises reflection angles (degrees 2-theta±0.2 degrees 2-theta), line spacings (d values), and relative intensities as shown in Table 5:

TABLE 5
XRPD Data for crystalline form
of Formula IB shown in Fig. 8

| Angle | | |
|---|---|---|
| (degrees 2- | | |
| theta ± 0.2 | | |
| degrees 2- | d Value | Relative |
| theta) | (Å) | Intensity |
| 4.94 | 17.875 | 44 |
| 7.13 | 12.385 | 32 |
| 8.32 | 10.614 | 20 |
| 10.01 | 8.829 | 15 |
| 10.45 | 8.461 | 15 |
| 11.39 | 7.765 | 100 |
| 11.65 | 7.587 | 70 |
| 11.97 | 7.388 | 15 |
| 12.42 | 7.120 | 25 |
| 13.61 | 6.499 | 43 |
| 14.31 | 6.185 | 22 |
| 14.92 | 5.934 | 15 |
| 15.13 | 5.852 | 49 |
| 16.49 | 5.371 | 27 |
| 16.72 | 5.299 | 71 |
| 16.88 | 5.248 | 33 |
| 17.04 | 5.198 | 49 |
| 17.94 | 4.941 | 15 |
| 18.48 | 4.798 | 17 |
| 18.80 | 4.717 | 15 |
| 19.12 | 4.637 | 21 |
| 19.67 | 4.509 | 15 |
| 19.89 | 4.459 | 21 |
| 20.31 | 4.368 | 30 |
| 20.54 | 4.320 | 19 |
| 21.02 | 4.223 | 75 |
| 22.35 | 3.974 | 60 |
| 23.05 | 3.856 | 47 |
| 23.47 | 3.788 | 32 |
| 23.84 | 3.730 | 35 |
| 24.40 | 3.645 | 19 |
| 24.78 | 3.590 | 18 |
| 25.02 | 3.557 | 13 |
| 25.55 | 3.483 | 14 |
| 26.80 | 3.324 | 14 |
| 27.32 | 3.262 | 19 |
| 28.39 | 3.141 | 12 |
| 28.87 | 3.090 | 15 |
| 29.07 | 3.070 | 19 |
| 29.30 | 3.045 | 19 |
| 29.69 | 3.006 | 32 |

In some embodiments of the present disclosure, the crystalline form of Formula IB is characterized by an XRPD pattern comprising a peak at one of the angles listed in Table 5. In other aspects, the crystalline form of Formula IB is characterized by an XRPD pattern comprising more than one peak at one of the angles listed in Table 5 above. In other aspects, the crystalline form of Formula IB is characterized by an XRPD pattern comprising two peaks selected from the angles listed in Table 5 above. In other aspects, the crystalline form of Formula IB is characterized by an XRPD pattern comprising three peaks selected from the angles listed in Table 5 above. In other aspects, the crystalline form of Formula IB is characterized by an XRPD pattern comprising four peaks selected from the angles listed in Table 5 above. In other aspects, the crystalline form of Formula IB is characterized by an XRPD pattern comprising five peaks selected from the angles listed in Table 5 above. In other aspects, the crystalline form of Formula IB is characterized by an XRPD pattern comprising six peaks selected from the angles listed in Table 5 above. In other aspects, the crystalline form of Formula IB is characterized by an XRPD pattern comprising seven peaks selected from the angles listed in Table 5 above. In other aspects, the crystalline form of Formula IB is characterized by an XRPD pattern comprising eight peaks selected from the angles listed in Table 5 above. In other aspects, the crystalline form of Formula IB is characterized by an XRPD pattern comprising nine peaks selected from the angles listed in Table 5 above. In other aspects, the crystalline form of Formula IB is characterized by an XRPD pattern comprising ten peaks selected from the angles listed in Table 5 above. In other aspects, the crystalline form of Formula IB is characterized by an XRPD pattern comprising more than ten peaks selected from the angles listed in Table 5 above.

In some embodiments, the crystalline form of Formula IB is characterized by an XRPD pattern comprising a peak at 11.4 degrees±0.2 degrees 2-theta. In other embodiments, the crystalline form of Formula IB is characterized by an XRPD pattern comprising peaks at 11.4, 11.6, 15.1, and 16.7 degrees±0.2 degrees 2-theta. In other embodiments, the crystalline form of Formula IB is characterized by an XRPD pattern comprising peaks at 4.9, 11.4, 11.6, and 15.1 degrees±0.2 degree 2-theta. In other embodiments, the crystalline form of Formula IB is characterized by an XRPD pattern comprising peaks at 4.9, 11.4, 11.6, 15.1, and 16.7 degrees±0.2 degree 2-theta. In other embodiments, the crystalline form of Formula IB is characterized by an XRPD pattern comprising peaks at 4.9, 11.4, 11.6, 15.1, 16.7, and 21.0 degrees±0.2 degree 2-theta. In yet other embodiments, the crystalline form of Formula IB is characterized by an XRPD pattern comprising peaks at 4.9, 11.4, 11.6, 15.1, 16.7, 21.0, and 22.4 degrees±0.2 degree 2-theta. In yet other embodiments, the crystalline form of Formula IB is characterized by an XRPD pattern comprising peaks at 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degree 2-theta.

In some embodiments of the present disclosure, the crystalline form of Formula IB is characterized by an XRPD pattern comprising peaks at three or more of 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degrees 2-theta. In some embodiments of the present disclosure, the crystalline form of Formula IB is characterized by an XRPD pattern comprising peaks at four or more of 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degrees 2-theta. In some embodiments of the present disclosure, the crystalline form of Formula IB is characterized by an XRPD pattern comprising peaks at five or more of 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degrees 2-theta. In some embodiments of the present disclosure, the crystalline form of Formula IB is characterized by an XRPD pattern comprising peaks at six or more of 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degrees 2-theta. In some embodiments of the present disclosure, the crystalline form of Formula IB is characterized by an XRPD pattern comprising peaks at seven or more of 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degrees 2-theta.

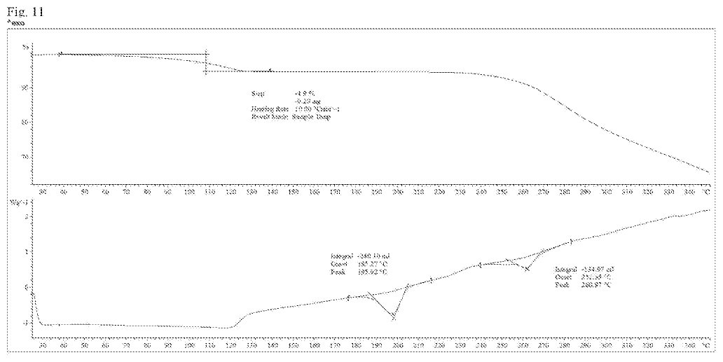

In some embodiments, the crystalline form of Formula IB can be characterized by a DSC thermogram and TGA profile substantially as shown in FIG. 11. As FIG. 11 shows, the crystalline form of Formula IB produced an endothermic peak at 195.92° C., with a peak onset temperature of 185.27° C., followed by an endothermic peak at 260.97° C. with a peak onset of 252.35° C., when heated at a rate of 10° C./min. In some embodiments of the present disclosure, the crystalline form of Formula IB is characterized by a DSC thermogram comprising an endothermic peak at about 196° C. when heated at a rate of 10° C./min. In other embodiments of the present disclosure, the crystalline form of Formula IB is characterized by a DSC thermogram comprising an endothermic peak at about 261° C. when heated at a rate of 10° C./min. As FIG. 11 shows, the crystalline form of Formula IB lost about 4.9% of its weight upon heating to about 150° C.

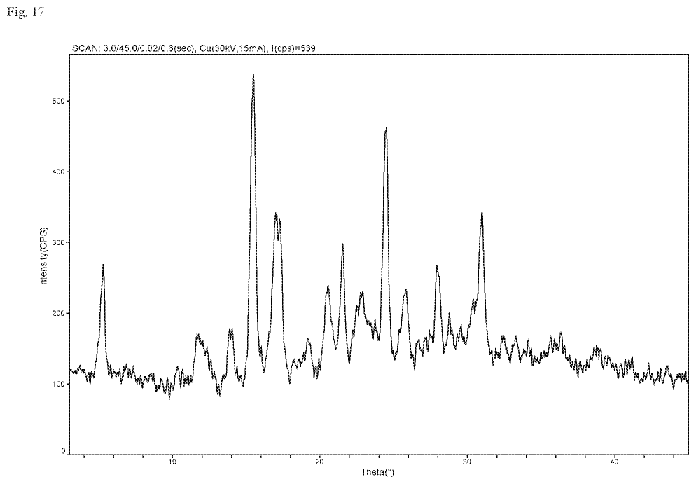

In some embodiments, a crystalline form of Formula IB exhibits an XRPD substantially as shown in FIG. 17. The XRPD of crystalline form of Formula IB shown in FIG. 17 comprises reflection angles (degrees 2-theta±0.2 degrees 2-theta), line spacings (d values), and relative intensities as shown in Table 5A:

TABLE 5A
XRPD Data for crystalline form
of Formula IB shown in Fig. 17

| Angle | | |
|---|---|---|
| (degrees 2- | | |
| theta ± 0.2 | | |
| degrees 2- | d Value | Relative |
| theta) | (Å) | Intensity |
| 5.301 | 16.6585 | 38.4 |
| 11.779 | 7.5067 | 18.3 |
| 14.04 | 6.3026 | 20.4 |
| 15.48 | 5.7193 | 100 |
| 17.261 | 5.1331 | 52.3 |
| 19.238 | 4.6098 | 12.7 |
| 20.56 | 4.3164 | 28.5 |
| 21.541 | 4.122 | 39.1 |
| 22.9 | 3.8803 | 19.9 |
| 24.5 | 3.6304 | 72.5 |
| 25.82 | 3.4476 | 24.1 |
| 27.999 | 3.1841 | 26.6 |
| 28.797 | 3.0977 | 10.9 |
| 30.98 | 2.8841 | 47 |
| 33.279 | 2.69 | 9.7 |

In some embodiments of the present disclosure, the crystalline form of Formula IB is characterized by an XRPD pattern comprising a peak at one of the angles listed in Table 5A. In other aspects, the crystalline form of Formula IB is characterized by an XRPD pattern comprising more than one peak at one of the angles listed in Table 5A above. In other aspects, the crystalline form of Formula IB is characterized by an XRPD pattern comprising two peaks selected from the angles listed in Table 5A above. In other aspects, the crystalline form of Formula IB is characterized by an XRPD pattern comprising three peaks selected from the angles listed in Table 5A above. In other aspects, the crystalline form of Formula IB is characterized by an XRPD pattern comprising four peaks selected from the angles listed in Table 5A above. In other aspects, the crystalline form of Formula IB is characterized by an XRPD pattern comprising five peaks selected from the angles listed in Table 5A above. In other aspects, the crystalline form of Formula IB is characterized by an XRPD pattern comprising six peaks selected from the angles listed in Table 5A above. In other aspects, the crystalline form of Formula IB is characterized by an XRPD pattern comprising seven peaks selected from the angles listed in Table 5A above. In other aspects, the crystalline form of Formula IB is characterized by an XRPD pattern comprising eight peaks selected from the angles listed in Table 5A above. In other aspects, the crystalline form of Formula IB is characterized by an XRPD pattern comprising nine peaks selected from the angles listed in Table 5A above. In other aspects, the crystalline form of Formula IB is characterized by an XRPD pattern comprising ten peaks selected from the angles listed in Table 5A above. In other aspects, the crystalline form of Formula IB is characterized by an XRPD pattern comprising more than ten peaks selected from the angles listed in Table 5A above.

In some embodiments, the crystalline form of Formula IB is characterized by an XRPD pattern comprising a peak at 5.3 and 15.5 degrees±0.2 degrees 2-theta. In other embodiments, the crystalline form of Formula IB is characterized by an XRPD pattern comprising peaks at 15.5 and 31.0 degrees±0.2 degrees 2-theta. In other embodiments, the crystalline form of Formula IB is characterized by an XRPD pattern comprising peaks at 15.5 and 24.5 degrees±0.2 degree 2-theta. In other embodiments, the crystalline form of Formula IB is characterized by an XRPD pattern comprising peaks at 15.5, 24.5, and 31.0 degrees±0.2 degree 2-theta. In other embodiments, the crystalline form of Formula IB is characterized by an XRPD pattern comprising peaks at 5.3, 15.5, 17.3, 24.5, and 31.0 degrees±0.2 degree 2-theta. In yet other embodiments, the crystalline form of Formula IB is characterized by an XRPD pattern comprising peaks at 5.3, 15.5, 17.3, 24.5, 28.0, and 31.0 degrees±0.2 degree 2-theta. In yet other embodiments, the crystalline form of Formula IB is characterized by an XRPD pattern comprising peaks at 5.3, 15.5, 17.3, 21.5, 24.5, 28.0, and 31.0 degrees±0.2 degree 2-theta.

In some embodiments of the present disclosure, the crystalline form of Formula IB is characterized by an XRPD pattern comprising peaks at three or more of 5.3, 15.5, 17.3, 21.5, 24.5, 28.0, and 31.0 degrees±0.2 degrees 2-theta. In some embodiments of the present disclosure, the crystalline form of Formula IB is characterized by an XRPD pattern comprising peaks at four or more of 5.3, 15.5, 17.3, 21.5, 24.5, 28.0, and 31.0 degrees±0.2 degrees 2-theta. In some embodiments of the present disclosure, the crystalline form of Formula IB is characterized by an XRPD pattern comprising peaks at five or more of 5.3, 15.5, 17.3, 21.5, 24.5, 28.0, and 31.0 degrees±0.2 degrees 2-theta. In some embodiments of the present disclosure, the crystalline form of Formula IB is characterized by an XRPD pattern comprising peaks at six or more of 5.3, 15.5, 17.3, 21.5, 24.5, 28.0, and 31.0 degrees±0.2 degrees 2-theta.

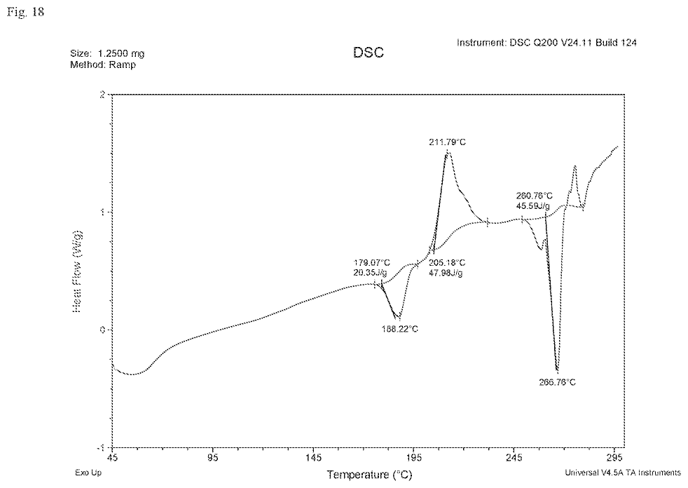

In some embodiments, the crystalline form of Formula IB can be characterized by a DSC thermogram substantially as shown in FIG. 18. As FIG. 18 shows, the crystalline form of Formula IB produced an endothermic peak at 188.22° C. (179.07° C. onset; 20.35 J/g), followed by an exothermic peak at 211.79° C. (205.18° C. onset; 47.98 J/g), followed by another endothermic peak at 266.76° C. (260.76° C. onset; 45.59 J/g), when heated at 10° C./min. In some embodiments of the present disclosure, the crystalline form of Formula IB is characterized by a DSC thermogram comprising an endothermic peak at about 188° C. when heated at a rate of 10° C./min. In other embodiments of the present disclosure, the crystalline form of Formula IB is characterized by a DSC thermogram comprising an endothermic peak at about 267° C. when heated at a rate of 10° C./min.

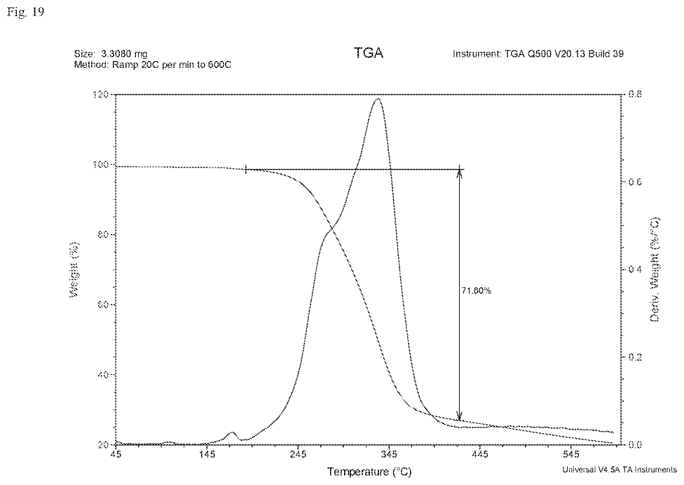

In some embodiments, the crystalline form of Formula IB can be characterized by a TGA profile substantially as shown in FIG. 19 when heated at a rate of 20° C./min.

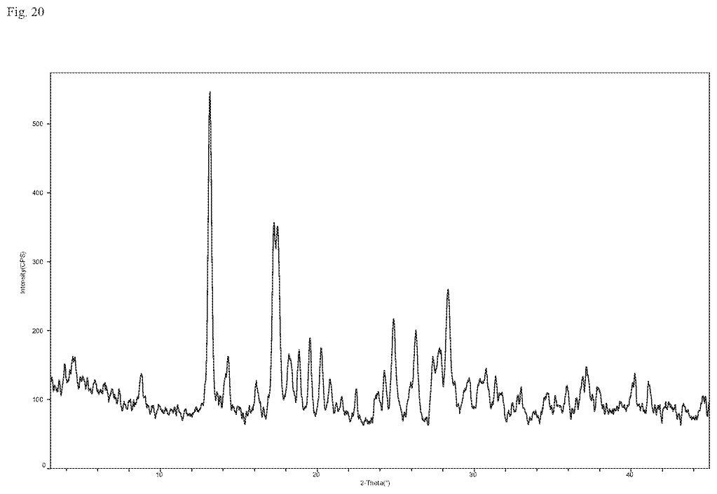

In some embodiments, the crystalline form of Formula IB (Form I) exhibits an XRPD substantially as shown in FIG. 20. The XRPD of Formula IB, Form I, shown in FIG. 20 comprises reflection angles (degrees 2-theta±0.2 degrees 2-theta), line spacings (d values), and relative intensities as shown in Table 5B:

TABLE 5B
XRPD Data for crystalline form
of Formula IB, Form I, shown in Fig. 20

| Angle | | |
|---|---|---|
| (degrees 2- | | |
| theta ± 0.2 | | |
| degrees 2- | d Value | Relative |
| theta) | (Å) | Intensity |
| 4.558 | 19.3693 | 11.3 |
| 8.819 | 10.0189 | 12.2 |
| 13.159 | 6.7223 | 100 |
| 14.302 | 6.1879 | 17.1 |
| 16.138 | 5.4877 | 10.9 |
| 17.46 | 5.075 | 57.6 |
| 18.239 | 4.86 | 17.1 |
| 18.84 | 4.7063 | 16.7 |
| 19.539 | 4.5394 | 23.3 |
| 20.241 | 4.3837 | 21 |
| 20.896 | 4.2477 | 9.6 |
| 22.48 | 3.9517 | 10.7 |
| 24.28 | 3.6628 | 16.1 |
| 24.86 | 3.5785 | 31.7 |
| 26.28 | 3.3883 | 28.9 |
| 27.741 | 3.2132 | 21.6 |
| 28.34 | 3.1466 | 39.4 |
| 29.68 | 3.0075 | 10.1 |
| 30.76 | 2.9043 | 13.9 |
| 31.78 | 2.8134 | 7.7 |
| 32.999 | 2.7122 | 11.3 |
| 34.64 | 2.5874 | 8.8 |
| 35.919 | 2.4981 | 9.9 |
| 37.142 | 2.4186 | 15.4 |
| 40.24 | 2.2392 | 12.4 |
| 41.178 | 2.1904 | 9.2 |

In some embodiments of the present disclosure, the crystalline form of Formula IB, Form I, is characterized by an XRPD pattern comprising a peak at one of the angles listed in Table 5B. In other aspects, the crystalline form of Formula IB, Form I, is characterized by an XRPD pattern comprising more than one peak at one of the angles listed in Table 5B above. In other aspects, the crystalline form of Formula IB, Form I, is characterized by an XRPD pattern comprising two peaks selected from the angles listed in Table 5B above. In other aspects, the crystalline form of Formula IB, Form I, is characterized by an XRPD pattern comprising three peaks selected from the angles listed in Table 5B above. In other aspects, the crystalline form of Formula IB, Form I, is characterized by an XRPD pattern comprising four peaks selected from the angles listed in Table 5B above. In other aspects, the crystalline form of Formula IB, Form I, is characterized by an XRPD pattern comprising five peaks selected from the angles listed in Table 5B above. In other aspects, the crystalline form of Formula IB, Form I, is characterized by an XRPD pattern comprising six peaks selected from the angles listed in Table 5B above. In other aspects, the crystalline form of Formula IB, Form I, is characterized by an XRPD pattern comprising seven peaks selected from the angles listed in Table 5B above. In other aspects, the crystalline form of Formula IB, Form I, is characterized by an XRPD pattern comprising eight peaks selected from the angles listed in Table 5B above. In other aspects, the crystalline form of Formula IB, Form I, is characterized by an XRPD pattern comprising nine peaks selected from the angles listed in Table 5B above. In other aspects, the crystalline form of Formula IB, Form I, is characterized by an XRPD pattern comprising ten peaks selected from the angles listed in Table 5B above. In other aspects, the crystalline form of Formula IB, Form I, is characterized by an XRPD pattern comprising more than ten peaks selected from the angles listed in Table 5B above.

In some embodiments, the crystalline form of Formula IB, Form I, is characterized by an XRPD pattern comprising peaks at 13.2 and 17.5 degrees±0.2 degree 2-theta. In other embodiments, the crystalline form of Formula IB, Form I, is characterized by an XRPD pattern comprising peaks at 13.2, 17.5, 26.3, and 28.3 degrees±0.2 degree 2-theta. In other embodiments, the crystalline form of Formula IB, Form I, is characterized by an XRPD pattern comprising peaks at 13.2, 17.5, 18.8, 19.5, and 20.2 degrees±0.2 degree 2-theta. In yet other embodiments, the crystalline form of Formula IB, Form I, is characterized by an XRPD pattern comprising peaks at 13.2, 17.5, 24.9, 26.3, and 28.3 degrees±0.2 degree 2-theta. In yet other embodiments, the crystalline form of Formula IB, Form I, is characterized by an XRPD pattern comprising peaks at 13.2, 17.5, 18.8, 19.5, 20.2, 24.9, 26.3, and 28.3 degrees±0.2 degree 2-theta.

In some embodiments of the present disclosure, the crystalline form of Formula IB, Form I, is characterized by an XRPD pattern comprising peaks at three or more of 13.2, 17.5, 18.8, 19.5, 20.2, 24.9, 26.3, and 28.3 degrees±0.2 degrees 2-theta. In some embodiments of the present disclosure, the crystalline form of Formula IB, Form I, is characterized by an XRPD pattern comprising peaks at four or more of 13.2, 17.5, 18.8, 19.5, 20.2, 24.9, 26.3, and 28.3 degrees±0.2 degrees 2-theta. In some embodiments of the present disclosure, the crystalline form of Formula IB, Form I, is characterized by an XRPD pattern comprising peaks at five or more of 13.2, 17.5, 18.8, 19.5, 20.2, 24.9, 26.3, and 28.3 degrees±0.2 degrees 2-theta. In some embodiments of the present disclosure, the crystalline form of Formula IB, Form I, is characterized by an XRPD pattern comprising peaks at six or more of 13.2, 17.5, 18.8, 19.5, 20.2, 24.9, 26.3, and 28.3 degrees±0.2 degrees 2-theta.

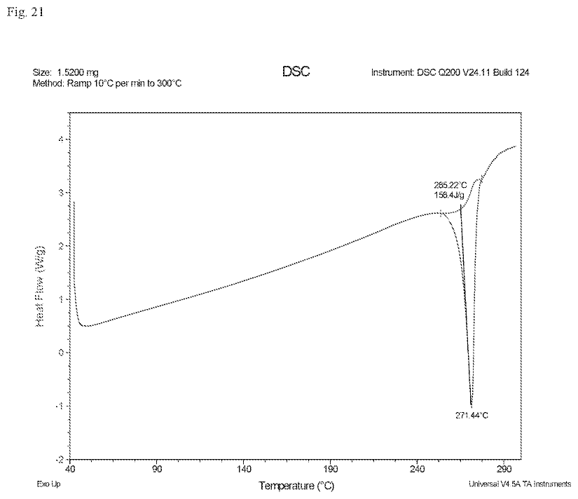

In some embodiments, the crystalline form of Formula IB can be characterized by a DSC thermogram substantially as shown in FIG. 21. As FIG. 21 shows, the crystalline form of Formula IB produced an endothermic peak at 271.44° C. (265.22° C. onset; 156.4 J/g) when heated at 10° C./min. In some embodiments of the present disclosure, the crystalline form of Formula IB is characterized by a DSC thermogram comprising an endothermic peak at about 271° C. when heated at a rate of 10° C./min.

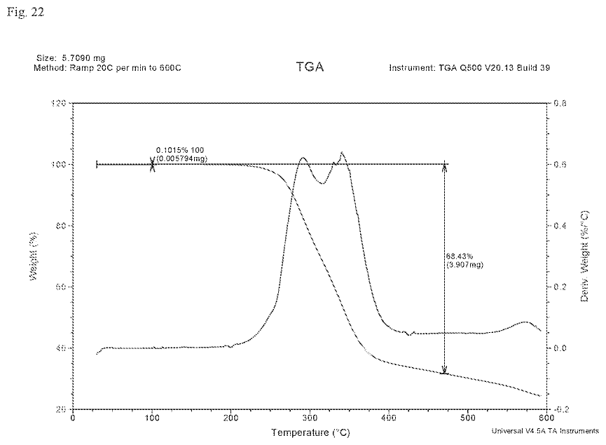

In some embodiments, the crystalline form of Formula IB, Form I can be characterized by a TGA profile substantially as shown in FIG. 22 when heated at a rate of 20° C./min.

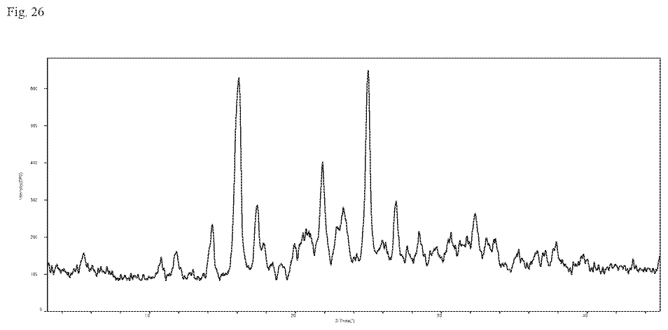

In some embodiments, the crystalline form of Formula IB (Form II) exhibits an XRPD substantially as shown in FIG. 26. The XRPD of Formula IB, Form I, shown in FIG. 26 comprises reflection angles (degrees 2-theta±0.2 degrees 2-theta), line spacings (d values), and relative intensities as shown in Table 5C:

TABLE 5C
XRPD Data for crystalline form of Formula IB,
Form II, shown in FIG. 26

| Angle | | |
|---|---|---|
| (degrees 2- | | |
| theta ± 0.2 | d Value | Relative |
| degrees 2-theta) | (Å) | Intensity |
| 5.499 | 16.0566 | 11.2 |
| 10.799 | 8.186 | 11.6 |
| 11.84 | 7.4685 | 14.2 |
| 14.3 | 6.1887 | 28.4 |
| 16.12 | 5.4938 | 100 |
| 17.38 | 5.0983 | 34.3 |
| 19.059 | 4.6528 | 7.8 |
| 19.938 | 4.4494 | 9.5 |
| 20.919 | 4.243 | 20.8 |
| 21.86 | 4.0625 | 49.6 |
| 23.359 | 3.8051 | 25.2 |
| 24.98 | 3.5617 | 97.2 |
| 26.9 | 3.3117 | 30.5 |
| 28.536 | 3.1254 | 14.4 |
| 30.659 | 2.9136 | 13.1 |
| 32.32 | 2.7676 | 22.3 |
| 33.118 | 2.7027 | 11 |
| 33.738 | 2.6545 | 12.5 |
| 35.3 | 2.5405 | 11 |
| 36.581 | 2.4544 | 8.3 |
| 37.141 | 2.4187 | 9.3 |
| 37.88 | 2.3732 | 14.4 |

In some embodiments of the present disclosure, the crystalline form of Formula IB, Form II, is characterized by an XRPD pattern comprising a peak at one of the angles listed in Table 5C. In other aspects, the crystalline form of Formula IB, Form II, is characterized by an XRPD pattern comprising more than one peak at one of the angles listed in Table 5C above. In other aspects, the crystalline form of Formula IB, Form II, is characterized by an XRPD pattern comprising two peaks selected from the angles listed in Table 5C above. In other aspects, the crystalline form of Formula IB, Form II, is characterized by an XRPD pattern comprising three peaks selected from the angles listed in Table 5C above. In other aspects, the crystalline form of Formula IB, Form II, is characterized by an XRPD pattern comprising four peaks selected from the angles listed in Table 5C above. In other aspects, the crystalline form of Formula IB, Form II, is characterized by an XRPD pattern comprising five peaks selected from the angles listed in Table 5C above. In other aspects, the crystalline form of Formula IB, Form II, is characterized by an XRPD pattern comprising six peaks selected from the angles listed in Table 5C above. In other aspects, the crystalline form of Formula IB, Form II, is characterized by an XRPD pattern comprising seven peaks selected from the angles listed in Table 5C above. In other aspects, the crystalline form of Formula IB, Form II, is characterized by an XRPD pattern comprising eight peaks selected from the angles listed in Table 5C above. In other aspects, the crystalline form of Formula IB, Form II, is characterized by an XRPD pattern comprising nine peaks selected from the angles listed in Table 5C above. In other aspects, the crystalline form of Formula IB, Form II, is characterized by an XRPD pattern comprising ten peaks selected from the angles listed in Table 5C above. In other aspects, the crystalline form of Formula IB, Form II, is characterized by an XRPD pattern comprising more than ten peaks selected from the angles listed in Table 5C above.

In some embodiments, the crystalline form of Formula IB, Form II, is characterized by an XRPD pattern comprising peaks at 16.1 and 25.0 degrees±0.2 degree 2-theta. In other embodiments, the crystalline form of Formula IB, Form II, is characterized by an XRPD pattern comprising peaks at 14.3, 16.1, 17.4, and 21.9 degrees±0.2 degree 2-theta. In other embodiments, the crystalline form of Formula IB, Form II, is characterized by an XRPD pattern comprising peaks at 14.3, 16.1, 17.4, 21.9, and 25.0 degrees±0.2 degree 2-theta. In yet other embodiments, the crystalline form of Formula IB, Form II, is characterized by an XRPD pattern comprising peaks at 14.3, 16.1, 17.4, 21.9, 25.0, and 26.9 degrees±0.2 degree 2-theta. In yet other embodiments, the crystalline form of Formula IB, Form II, is characterized by an XRPD pattern comprising peaks at 14.3, 16.1, 17.4, 21.9, 25.0, 26.9, and 32.3 degrees±0.2 degree 2-theta.

In some embodiments of the present disclosure, the crystalline form of Formula IB, Form II, is characterized by an XRPD pattern comprising peaks at three or more of 14.3, 16.1, 17.4, 21.9, 25.0, 26.9, and 32.3 degrees±0.2 degrees 2-theta. In some embodiments of the present disclosure, the crystalline form of Formula IB, Form II, is characterized by an XRPD pattern comprising peaks at four or more of 14.3, 16.1, 17.4, 21.9, 25.0, 26.9, and 32.3 degrees±0.2 degrees 2-theta. In some embodiments of the present disclosure, the crystalline form of Formula IB, Form II, is characterized by an XRPD pattern comprising peaks at five or more of 14.3, 16.1, 17.4, 21.9, 25.0, 26.9, and 32.3 degrees±0.2 degrees 2-theta. In some embodiments of the present disclosure, the crystalline form of Formula IB, Form II, is characterized by an XRPD pattern comprising peaks at six or more of 14.3, 16.1, 17.4, 21.9, 25.0, 26.9, and 32.3 degrees±0.2 degrees 2-theta.

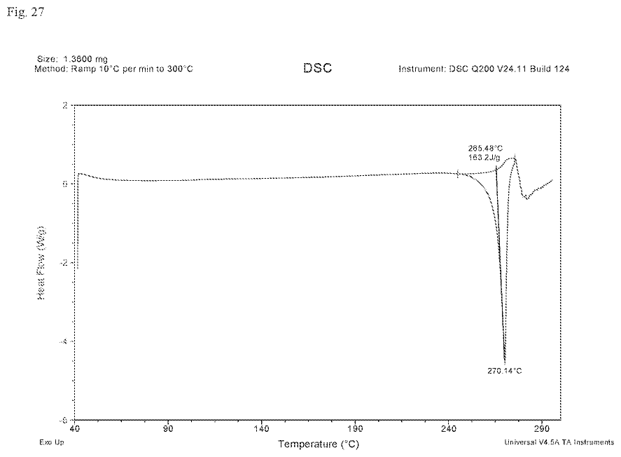

In some embodiments, the crystalline form of Formula IB, Form II, can be characterized by a DSC thermogram substantially as shown in FIG. 27. As FIG. 27 shows, the crystalline form of Formula IB, Form II, produced an endothermic peak at 270.14° C. (265.48° C. onset; 163.2 J/g) when heated at 10° C./min. In some embodiments of the present disclosure, the crystalline form of Formula IB is characterized by a DSC thermogram comprising an endothermic peak at about 270° C. when heated at a rate of 10° C./min.

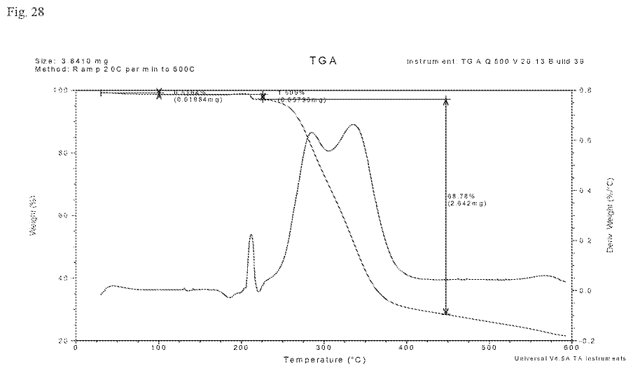

In some embodiments, the crystalline form of Formula IB, Form II can be characterized by a TGA profile substantially as shown in FIG. 28 when heated at a rate of 20° C./min.

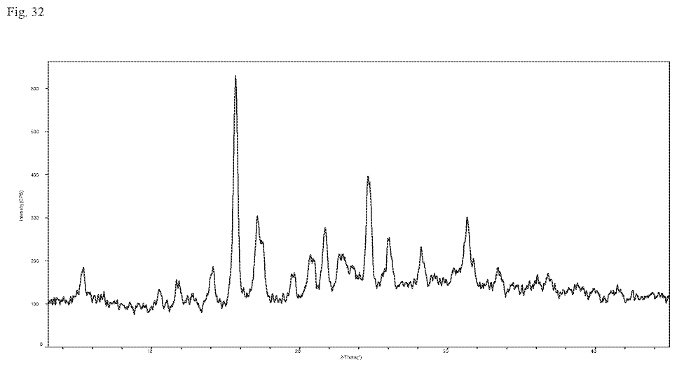

In some embodiments, a crystalline form of Formula IB exhibits an XRPD substantially as shown in FIG. 32. The XRPD of crystalline form of Formula IB, Form III shown in FIG. 32 comprises reflection angles (degrees 2-theta±0.2 degrees 2-theta), line spacings (d values), and relative intensities as shown in Table 5D:

TABLE 5D
XRPD Data for crystalline form of Formula IB,
Form III, shown in FIG. 32

| Angle | | |
|---|---|---|
| (degrees 2- | | |
| theta ± 0.2 | d Value | Relative |
| degrees 2-theta) | (Å) | Intensity |
| 5.399 | 16.3551 | 16.5 |
| 10.5 | 8.4181 | 9.9 |
| 11.859 | 7.4563 | 13.5 |
| 12.8 | 6.9105 | 7.6 |
| 14.159 | 6.2498 | 18.8 |
| 15.68 | 5.6469 | 100 |
| 17.258 | 5.134 | 31.2 |
| 19.64 | 4.5164 | 11.4 |
| 20.778 | 4.2714 | 16.9 |
| 21.739 | 4.0847 | 27.9 |
| 22.9 | 3.8802 | 14.6 |
| 23.659 | 3.7574 | 9.7 |
| 24.62 | 3.6129 | 47.1 |
| 26.059 | 3.4165 | 22.2 |
| 28.238 | 3.1577 | 16.2 |
| 31.321 | 2.8536 | 30.4 |
| 33.542 | 2.6695 | 9.3 |
| 36.097 | 2.4862 | 8.9 |
| 36.782 | 2.4415 | 8.4 |

In some embodiments of the present disclosure, the crystalline form of Formula IB, Form III is characterized by an XRPD pattern comprising a peak at one of the angles listed in Table 5D. In other aspects, the crystalline form of Formula IB, Form III is characterized by an XRPD pattern comprising more than one peak at one of the angles listed in Table 5D above. In other aspects, the crystalline form of Formula IB, Form III is characterized by an XRPD pattern comprising two peaks selected from the angles listed in Table 5D above. In other aspects, the crystalline form of Formula IB, Form III is characterized by an XRPD pattern comprising three peaks selected from the angles listed in Table 5D above. In other aspects, the crystalline form of Formula IB, Form III is characterized by an XRPD pattern comprising four peaks selected from the angles listed in Table 5D above. In other aspects, the crystalline form of Formula IB, Form III is characterized by an XRPD pattern comprising five peaks selected from the angles listed in Table 5D above. In other aspects, the crystalline form of Formula IB, Form III is characterized by an XRPD pattern comprising six peaks selected from the angles listed in Table 5D above. In other aspects, the crystalline form of Formula IB, Form III is characterized by an XRPD pattern comprising seven peaks selected from the angles listed in Table 5D above. In other aspects, the crystalline form of Formula IB, Form III is characterized by an XRPD pattern comprising eight peaks selected from the angles listed in Table 5D above. In other aspects, the crystalline form of Formula IB, Form III is characterized by an XRPD pattern comprising nine peaks selected from the angles listed in Table 5D above. In other aspects, the crystalline form of Formula IB, Form III is characterized by an XRPD pattern comprising ten peaks selected from the angles listed in Table 5D above. In other aspects, the crystalline form of Formula IB, Form III is characterized by an XRPD pattern comprising more than ten peaks selected from the angles listed in Table 5D above.

In some embodiments, the crystalline form of Formula IB, Form III is characterized by an XRPD pattern comprising peaks at 15.7, 24.6, and 31.3 degrees±0.2 degree 2-theta. In other embodiments, the crystalline form of Formula IB, Form III is characterized by an XRPD pattern comprising peaks at 15.7, 17.3, 24.6, and 31.3 degrees±0.2 degree 2-theta. In other embodiments, the crystalline form of Formula IB, Form III is characterized by an XRPD pattern comprising peaks at 15.7, 17.3, 21.7, 24.6, and 31.3 degrees±0.2 degree 2-theta. In yet other embodiments, the crystalline form of Formula IB, Form III is characterized by an XRPD pattern comprising peaks at 15.7, 17.3, 21.7, 24.6, 26.1, 28.2, and 31.3 degrees±0.2 degree 2-theta. In yet other embodiments, the crystalline form of Formula IB, Form III is characterized by an XRPD pattern comprising peaks at 5.4, 15.7, 17.3, 21.7, 24.6, 26.1, 28.2, and 31.3 degrees±0.2 degree 2-theta.

In some embodiments of the present disclosure, the crystalline form of Formula IB, Form III is characterized by an XRPD pattern comprising peaks at three or more of 5.4, 15.7, 17.3, 21.7, 24.6, 26.1, 28.2, and 31.3 degrees±0.2 degrees 2-theta. In some embodiments of the present disclosure, the crystalline form of Formula IB, Form III is characterized by an XRPD pattern comprising peaks at four or more of 5.4, 15.7, 17.3, 21.7, 24.6, 26.1, 28.2, and 31.3 degrees±0.2 degrees 2-theta. In some embodiments of the present disclosure, the crystalline form of Formula IB, Form III is characterized by an XRPD pattern comprising peaks at five or more of 5.4, 15.7, 17.3, 21.7, 24.6, 26.1, 28.2, and 31.3 degrees±0.2 degrees 2-theta. In some embodiments of the present disclosure, the crystalline form of Formula IB, Form III is characterized by an XRPD pattern comprising peaks at six or more of 5.4, 15.7, 17.3, 21.7, 24.6, 26.1, 28.2, and 31.3 degrees±0.2 degrees 2-theta.

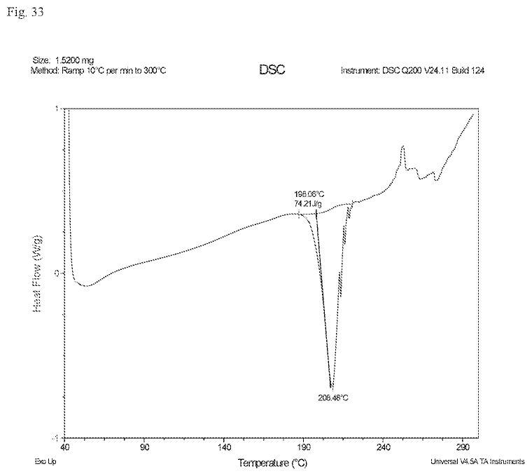

In some embodiments, the crystalline form of Formula IB, Form III, can be characterized by a DSC thermogram substantially as shown in FIG. 33. As FIG. 33 shows, the crystalline form of Formula IB, Form III, produced an endothermic peak at 208.48° C. (198.06° C. onset; 74.21 J/g) when heated at 10° C./min. In some embodiments of the present disclosure, the crystalline form of Formula IB, Form III, is characterized by a DSC thermogram comprising an endothermic peak at about 208° C. when heated at a rate of 10° C./min.

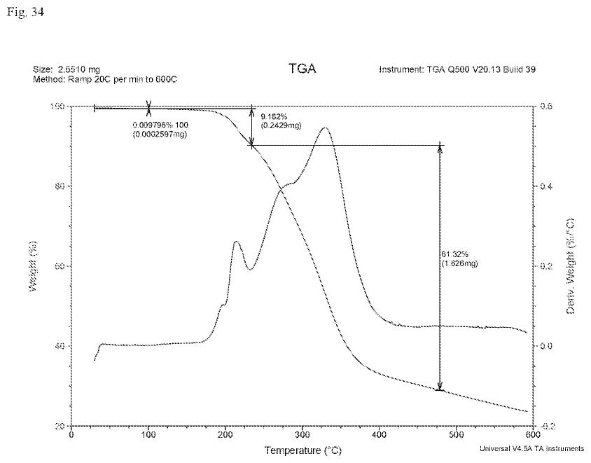

In some embodiments, the crystalline form of Formula IB, Form III can be characterized by a TGA profile substantially as shown in FIG. 34 when heated at a rate of 20° C./min.

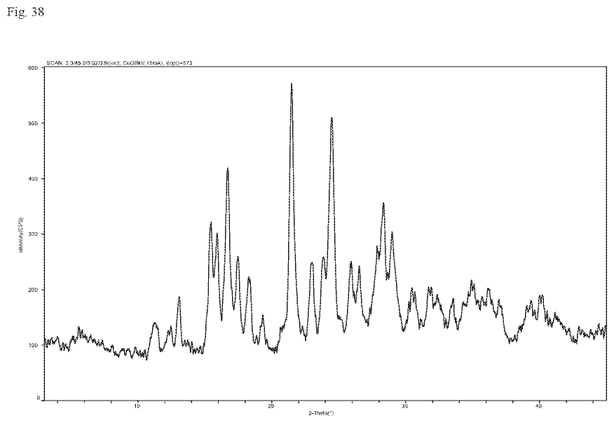

In some embodiments, a crystalline form of Formula IB exhibits an XRPD substantially as shown in FIG. 38. The XRPD of crystalline form of Formula IB, Form IV shown in FIG. 38 comprises reflection angles (degrees 2-theta±0.2 degrees 2-theta), line spacings (d values), and relative intensities as shown in Table 5E:

TABLE 5E
XRPD Data for crystalline form of Formula IB,
Form IV, shown in FIG. 38

| Angle | | |
|---|---|---|
| (degrees 2- | | |
| theta ± 0.2 | d Value | Relative |
| degrees 2-theta) | (Å) | Intensity |
| 11.319 | 7.8109 | 11.8 |
| 12.461 | 7.0977 | 9.1 |
| 13.061 | 6.7727 | 21.1 |
| 15.459 | 5.727 | 42.1 |
| 15.919 | 5.5626 | 35.9 |
| 16.7 | 5.3042 | 59.4 |
| 17.46 | 5.0749 | 28.3 |
| 18.28 | 4.8493 | 22.6 |
| 19.357 | 4.5818 | 10.1 |
| 21.48 | 4.1334 | 100 |
| 22.96 | 3.8702 | 27.9 |
| 23.84 | 3.7293 | 29.8 |
| 24.5 | 3.6304 | 81.6 |
| 25.94 | 3.432 | 26.2 |
| 26.557 | 3.3536 | 22.4 |
| 28.34 | 3.1466 | 48 |
| 28.98 | 3.0785 | 34.2 |
| 30.681 | 2.9116 | 14 |
| 31.882 | 2.8046 | 15 |
| 33.558 | 2.6682 | 11.6 |
| 34.918 | 2.5674 | 14.4 |
| 36.258 | 2.4755 | 12.1 |
| 40.181 | 2.2424 | 11.8 |

In some embodiments of the present disclosure, the crystalline form of Formula IB, Form IV is characterized by an XRPD pattern comprising a peak at one of the angles listed in Table 5E. In other aspects, the crystalline form of Formula IB, Form IV is characterized by an XRPD pattern comprising more than one peak at one of the angles listed in Table 5E above. In other aspects, the crystalline form of Formula IB, Form IV is characterized by an XRPD pattern comprising two peaks selected from the angles listed in Table 5E above. In other aspects, the crystalline form of Formula IB, Form IV is characterized by an XRPD pattern comprising three peaks selected from the angles listed in Table 5E above. In other aspects, the crystalline form of Formula IB, Form IV is characterized by an XRPD pattern comprising four peaks selected from the angles listed in Table 5E above. In other aspects, the crystalline form of Formula IB, Form IV is characterized by an XRPD pattern comprising five peaks selected from the angles listed in Table 5E above. In other aspects, the crystalline form of Formula IB, Form IV is characterized by an XRPD pattern comprising six peaks selected from the angles listed in Table 5E above. In other aspects, the crystalline form of Formula IB, Form IV is characterized by an XRPD pattern comprising seven peaks selected from the angles listed in Table 5E above. In other aspects, the crystalline form of Formula IB, Form IV is characterized by an XRPD pattern comprising eight peaks selected from the angles listed in Table 5E above. In other aspects, the crystalline form of Formula IB, Form IV is characterized by an XRPD pattern comprising nine peaks selected from the angles listed in Table 5E above. In other aspects, the crystalline form of Formula IB, Form IV is characterized by an XRPD pattern comprising ten peaks selected from the angles listed in Table 5E above. In other aspects, the crystalline form of Formula IB, Form IV is characterized by an XRPD pattern comprising more than ten peaks selected from the angles listed in Table 5E above.

In some embodiments, the crystalline form of Formula IB, Form IV is characterized by an XRPD pattern comprising peaks at 15.9, 21.5, and 24.5 degrees±0.2 degree 2-theta. In other embodiments, the crystalline form of Formula IB, Form IV is characterized by an XRPD pattern comprising peaks at 15.5, 15.9, 16.7, 17.5, and 21.5 degrees±0.2 degree 2-theta. In other embodiments, the crystalline form of Formula IB, Form IV is characterized by an XRPD pattern comprising peaks at 15.5, 15.9, 16.7, 17.5, 21.5, 23.0, and 24.5 degrees±0.2 degree 2-theta. In yet other embodiments, the crystalline form of Formula IB, Form IV is characterized by an XRPD pattern comprising peaks at 13.1, 15.5, 15.9, 16.7, 17.5, 21.5, 23.0, 24.5, and 28.3 degrees±0.2 degree 2-theta. In yet other embodiments, the crystalline form of Formula IB, Form IV is characterized by an XRPD pattern comprising peaks at 13.1, 15.5, 15.9, 16.7, 17.5, 21.5, 23.0, 24.5, 28.3, and 29.0 degrees±0.2 degree 2-theta.

In some embodiments of the present disclosure, the crystalline form of Formula IB, Form IV is characterized by an XRPD pattern comprising peaks at three or more of 13.1, 15.5, 15.9, 16.7, 17.5, 21.5, 23.0, 24.5, 28.3, and 29.0 degrees±0.2 degrees 2-theta. In some embodiments of the present disclosure, the crystalline form of Formula IB, Form IV is characterized by an XRPD pattern comprising peaks at four or more of 13.1, 15.5, 15.9, 16.7, 17.5, 21.5, 23.0, 24.5, 28.3, and 29.0 degrees±0.2 degrees 2-theta. In some embodiments of the present disclosure, the crystalline form of Formula IB, Form IV is characterized by an XRPD pattern comprising peaks at five or more of 13.1, 15.5, 15.9, 16.7, 17.5, 21.5, 23.0, 24.5, 28.3, and 29.0 degrees±0.2 degrees 2-theta. In some embodiments of the present disclosure, the crystalline form of Formula IB, Form IV is characterized by an XRPD pattern comprising peaks at six or more of 13.1, 15.5, 15.9, 16.7, 17.5, 21.5, 23.0, 24.5, 28.3, and 29.0 degrees±0.2 degrees 2-theta.

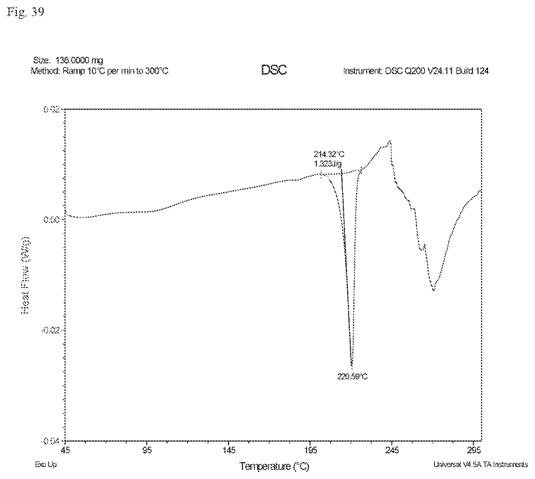

In some embodiments, the crystalline form of Formula IB, Form IV, can be characterized by a DSC thermogram substantially as shown in FIG. 39. As FIG. 39 shows, the crystalline form of Formula IB, Form IV, produced an endothermic peak at 220.59° C. (214.32° C. onset; 1.323 J/g) when heated at 10° C./min. In some embodiments of the present disclosure, the crystalline form of Formula IB, Form IV, is characterized by a DSC thermogram comprising an endothermic peak at about 221° C. when heated at a rate of 10° C./min.

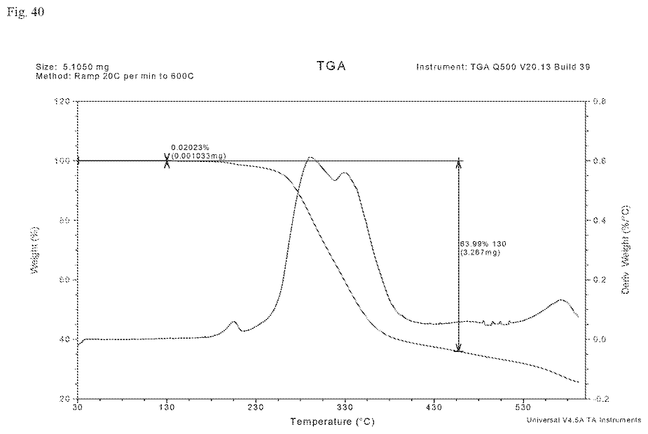

In some embodiments, the crystalline form of Formula IB, Form IV can be characterized by a TGA profile substantially as shown in FIG. 40 when heated at a rate of 20° C./min.

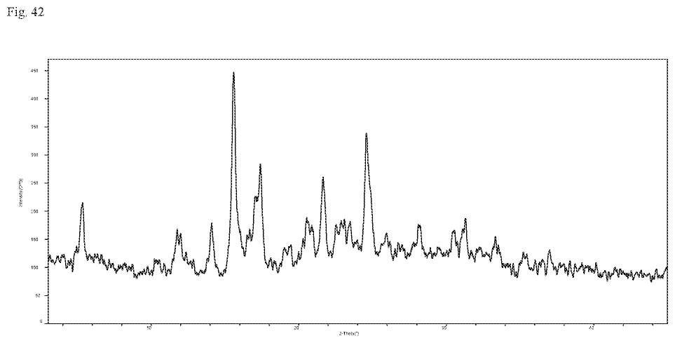

In some embodiments, a crystalline form of Formula IB exhibits an XRPD substantially as shown in FIG. 42. The XRPD of crystalline form of Formula IB shown in FIG. 42 comprises reflection angles (degrees 2-theta±0.2 degrees 2-theta), line spacings (d values), and relative intensities as shown in Table 5F:

TABLE 5F
XRPD Data for crystalline form of Formula IB
shown in FIG. 42

| Angle | | |
|---|---|---|
| (degrees 2- | | |
| theta ± 0.2 | d Value | Relative |
| degrees 2-theta) | (Å) | Intensity |
| 5.34 | 16.5357 | 33.8 |
| 11.962 | 7.3924 | 21.3 |
| 14.118 | 6.268 | 25.9 |
| 15.581 | 5.6827 | 100 |
| 17.4 | 5.0924 | 51.6 |
| 19.039 | 4.6575 | 11.4 |
| 19.46 | 4.5578 | 12 |
| 20.88 | 4.2508 | 19 |
| 21.641 | 4.1031 | 41.7 |
| 22.823 | 3.8933 | 16.3 |
| 23.083 | 3.85 | 18.1 |
| 23.48 | 3.7857 | 16 |
| 23.516 | 3.78 | 14.3 |
| 24.6 | 3.6159 | 60.6 |
| 28.219 | 3.1597 | 16.6 |
| 30.54 | 2.9247 | 15.2 |
| 31.3 | 2.8554 | 20.7 |
| 33.357 | 2.6839 | 15.2 |
| 35.459 | 2.5295 | 11.4 |
| 37.037 | 2.4252 | 10.2 |

In some embodiments of the present disclosure, the crystalline form of Formula IB is characterized by an XRPD pattern comprising a peak at one of the angles listed in Table 5F. In other aspects, the crystalline form of Formula IB is characterized by an XRPD pattern comprising more than one peak at one of the angles listed in Table 5F above. In other aspects, the crystalline form of Formula IB is characterized by an XRPD pattern comprising two peaks selected from the angles listed in Table 5F above. In other aspects, the crystalline form of Formula IB is characterized by an XRPD pattern comprising three peaks selected from the angles listed in Table 5F above. In other aspects, the crystalline form of Formula IB is characterized by an XRPD pattern comprising four peaks selected from the angles listed in Table 5F above. In other aspects, the crystalline form of Formula IB is characterized by an XRPD pattern comprising five peaks selected from the angles listed in Table 5F above. In other aspects, the crystalline form of Formula IB is characterized by an XRPD pattern comprising six peaks selected from the angles listed in Table 5F above. In other aspects, the crystalline form of Formula IB is characterized by an XRPD pattern comprising seven peaks selected from the angles listed in Table 5F above. In other aspects, the crystalline form of Formula IB is characterized by an XRPD pattern comprising eight peaks selected from the angles listed in Table 5F above. In other aspects, the crystalline form of Formula IB is characterized by an XRPD pattern comprising nine peaks selected from the angles listed in Table 5F above. In other aspects, the crystalline form of Formula IB is characterized by an XRPD pattern comprising ten peaks selected from the angles listed in Table 5F above. In other aspects, the crystalline form of Formula IB is characterized by an XRPD pattern comprising more than ten peaks selected from the angles listed in Table 5F above.

In some embodiments, the crystalline form of Formula IB is characterized by an XRPD pattern comprising peaks at 15.6 and 24.6 degrees±0.2 degree 2-theta. In other embodiments, the crystalline form of Formula IB is characterized by an XRPD pattern comprising peaks at 15.6, 17.4, and 21.6 degrees±0.2 degree 2-theta. In other embodiments, the crystalline form of Formula IB is characterized by an XRPD pattern comprising peaks at 15.6, 17.4, 21.6, and 24.6 degrees±0.2 degree 2-theta. In yet other embodiments, the crystalline form of Formula IB is characterized by an XRPD pattern comprising peaks at 14.1, 15.6, 17.4, 21.6, and 24.6 degrees±0.2 degree 2-theta. In yet other embodiments, the crystalline form of Formula IB is characterized by an XRPD pattern comprising peaks at 5.3, 14.1, 15.6, 17.4, 21.6, and 24.6 degrees±0.2 degree 2-theta.

In some embodiments of the present disclosure, the crystalline form of Formula IB is characterized by an XRPD pattern comprising peaks at two or more of 5.3, 14.1, 15.6, 17.4, 21.6, and 24.6 degrees±0.2 degrees 2-theta. In some embodiments of the present disclosure, the crystalline form of Formula IB is characterized by an XRPD pattern comprising peaks at three or more of 5.3, 14.1, 15.6, 17.4, 21.6, and 24.6 degrees±0.2 degrees 2-theta. In some embodiments of the present disclosure, the crystalline form of Formula IB is characterized by an XRPD pattern comprising peaks at four or more of 5.3, 14.1, 15.6, 17.4, 21.6, and 24.6 degrees±0.2 degrees 2-theta. In some embodiments of the present disclosure, the crystalline form of Formula IB is characterized by an XRPD pattern comprising peaks at five or more of 5.3, 14.1, 15.6, 17.4, 21.6, and 24.6 degrees±0.2 degrees 2-theta.

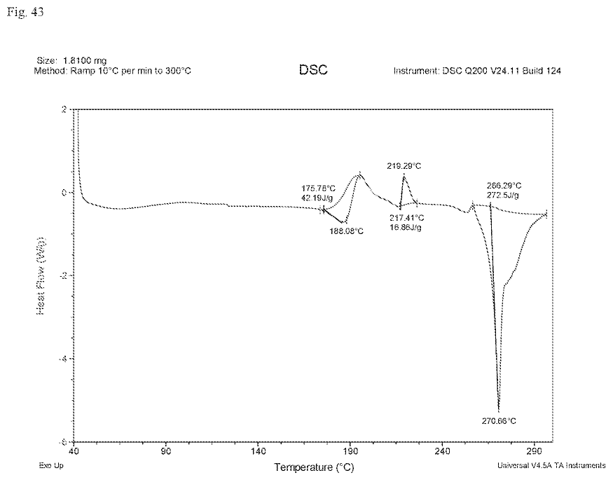

In some embodiments, the crystalline form of Formula IB, can be characterized by a DSC thermogram substantially as shown in FIG. 43. As FIG. 43 shows, the crystalline form of Formula IB, produced an endothermic peak at 188.08° C. (175.78° C. onset; 42.19 J/g), followed by an exothermic peak at 219.29° C. (217.41° C. onset; 16.86 J/g), followed by an endothermic peak at 270.66° C. (266.29° C. onset; 272.5 J/g) when heated at 10° C./min. In some embodiments of the present disclosure, the crystalline form of Formula IB, is characterized by a DSC thermogram comprising an endothermic peak at about 188° C. when heated at a rate of 10° C./min. In some embodiments of the present disclosure, the crystalline form of Formula IB, is characterized by a DSC thermogram comprising an endothermic peak at about 271° C. when heated at a rate of 10° C./min.

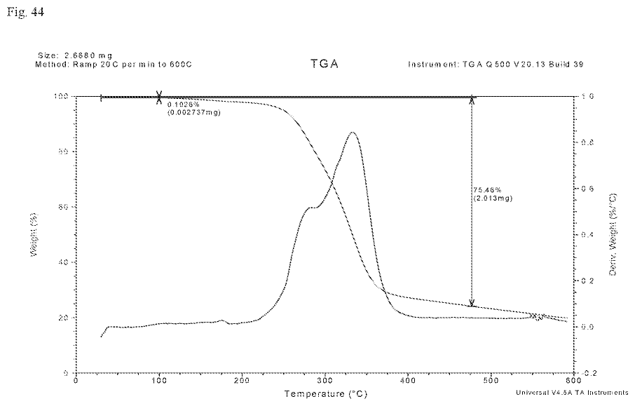

In some embodiments, the crystalline form of Formula IB can be characterized by a TGA profile substantially as shown in FIG. 44 when heated at a rate of 20° C./min.

### Formula IC—(Formula I Oxalate Salt)

In some aspects, the disclosure is directed to a crystalline form of the oxalate salt, i.e., Formula IC. In other aspects, the crystalline form of Formula IC is substantially free of any other solid form of Formula IC.

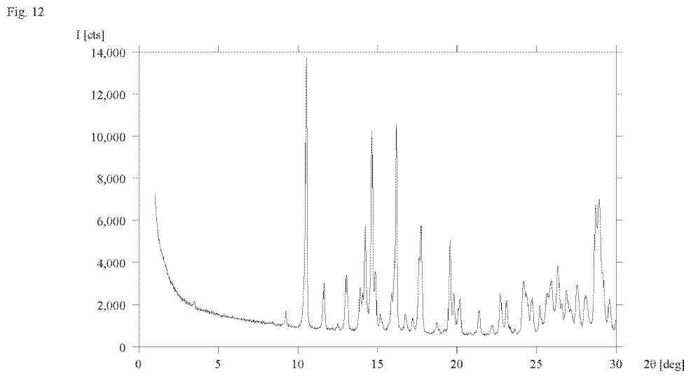

In some embodiments, the crystalline form of Formula IC exhibits an XRPD substantially as shown in FIG. 12. The XRPD of crystalline form of Formula IC shown in FIG. 12 comprises reflection angles (degrees 2-theta±0.2 degrees 2-theta), line spacings (d values), and relative intensities as shown in Table 6:

TABLE 6
XRPD Data for crystalline form of Formula IC
shown in FIG. 12

| Angle | | |
|---|---|---|
| (degrees 2- | | |
| theta ± 0.2 | d Value | Relative |
| degrees 2-theta) | (Å) | Intensity |
| 9.26 | 9.545 | 12 |
| 10.52 | 8.400 | 100 |
| 11.64 | 7.599 | 23 |
| 12.49 | 7.081 | 8 |
| 13.06 | 6.775 | 24 |
| 13.91 | 6.363 | 20 |
| 14.07 | 6.289 | 19 |
| 14.23 | 6.220 | 42 |
| 14.66 | 6.039 | 76 |
| 14.87 | 5.953 | 26 |
| 15.18 | 5.830 | 12 |
| 15.90 | 5.571 | 19 |
| 16.18 | 5.473 | 78 |
| 16.76 | 5.286 | 11 |
| 17.22 | 5.146 | 10 |
| 17.63 | 5.027 | 32 |
| 17.74 | 4.996 | 43 |
| 18.73 | 4.734 | 8 |
| 19.56 | 4.534 | 37 |
| 19.80 | 4.480 | 19 |
| 20.08 | 4.418 | 13 |
| 20.19 | 4.394 | 17 |
| 21.38 | 4.152 | 12 |
| 22.21 | 4.000 | 8 |
| 22.73 | 3.909 | 19 |
| 23.11 | 3.845 | 17 |
| 23.64 | 3.761 | 6 |
| 24.18 | 3.678 | 23 |
| 24.36 | 3.650 | 19 |
| 24.71 | 3.600 | 17 |
| 25.21 | 3.530 | 14 |
| 25.66 | 3.469 | 19 |
| 25.91 | 3.436 | 23 |
| 26.34 | 3.380 | 28 |
| 26.58 | 3.351 | 15 |
| 26.88 | 3.314 | 20 |
| 27.15 | 3.281 | 14 |
| 27.55 | 3.234 | 21 |
| 28.08 | 3.175 | 18 |
| 28.73 | 3.105 | 50 |
| 28.93 | 3.084 | 52 |
| 29.57 | 3.018 | 17 |

In some embodiments of the present disclosure, the crystalline form of Formula IC is characterized by an XRPD pattern comprising a peak at one of the angles listed in Table 6. In other aspects, the crystalline form of Formula IC is characterized by an XRPD pattern comprising more than one peak at one of the angles listed in Table 6 above. In other aspects, the crystalline form of Formula IC is characterized by an XRPD pattern comprising two peaks selected from the angles listed in Table 6 above. In other aspects, the crystalline form of Formula IC is characterized by an XRPD pattern comprising three peaks selected from the angles listed in Table 6 above. In other aspects, the crystalline form of Formula IC is characterized by an XRPD pattern comprising four peaks selected from the angles listed in Table 6 above. In other aspects, the crystalline form of Formula IC is characterized by an XRPD pattern comprising five peaks selected from the angles listed in Table 6 above. In other aspects, the crystalline form of Formula IC is characterized by an XRPD pattern comprising six peaks selected from the angles listed in Table 6 above. In other aspects, the crystalline form of Formula IC is characterized by an XRPD pattern comprising seven peaks selected from the angles listed in Table 6 above. In other aspects, the crystalline form of Formula IC is characterized by an XRPD pattern comprising eight peaks selected from the angles listed in Table 6 above. In other aspects, the crystalline form of Formula IC is characterized by an XRPD pattern comprising nine peaks selected from the angles listed in Table 6 above. In other aspects, the crystalline form of Formula IC is characterized by an XRPD pattern comprising ten peaks selected from the angles listed in Table 6 above. In other aspects, the crystalline form of Formula IC is characterized by an XRPD pattern comprising more than ten peaks selected from the angles listed in Table 6 above.

In some embodiments, the crystalline form of Formula IC is characterized by an XRPD pattern comprising a peak at 10.5 degrees±0.2 degrees 2-theta. In other embodiments, the crystalline form of Formula IC is characterized by an XRPD pattern comprising peaks at 10.5, 14.7, 16.2 degrees±0.2 degrees 2-theta. In other embodiments, the crystalline form of Formula IC is characterized by an XRPD pattern comprising peaks at 10.5, 14.7, 16.2, and 28.7 degrees±0.2 degree 2-theta. In other embodiments, the crystalline form of Formula IC is characterized by an XRPD pattern comprising peaks at 10.5, 14.7, 16.2, 17.6, 17.7, 19.6, 28.7, and 28.9 degrees±0.2 degree 2-theta. In other embodiments, the crystalline form of Formula IC is characterized by an XRPD pattern comprising peaks at 10.5, 14.2, 14.7, 28.7, and 28.9 degrees±0.2 degree 2-theta. In yet other embodiments, the crystalline form of Formula IC is characterized by an XRPD pattern comprising peaks at 10.5, 11.6, 13.1, 14.2, and 14.7 degrees±0.2 degree 2-theta. In yet other embodiments, the crystalline form of Formula IC is characterized by an XRPD pattern comprising peaks at 10.5, 11.6, 13.1, 14.2, 14.7, 14.9, 16.2, 17.6, 17.7, 19.6, 28.7, and 28.9 degrees±0.2 degree 2-theta.

In some embodiments of the present disclosure, the crystalline form of Formula IC is characterized by an XRPD pattern comprising peaks at three or more of 10.5, 11.6, 13.1, 14.2, 14.7, 14.9, 16.2, 17.6, 17.7, 19.6, 28.7, and 28.9 degrees±0.2 degrees 2-theta. In some embodiments of the present disclosure, the crystalline form of Formula IC is characterized by an XRPD pattern comprising peaks at four or more of 10.5, 11.6, 13.1, 14.2, 14.7, 14.9, 16.2, 17.6, 17.7, 19.6, 28.7, and 28.9 degrees±0.2 degrees 2-theta. In some embodiments of the present disclosure, the crystalline form of Formula IC is characterized by an XRPD pattern comprising peaks at five or more of 10.5, 11.6, 13.1, 14.2, 14.7, 14.9, 16.2, 17.6, 17.7, 19.6, 28.7, and 28.9 degrees±0.2 degrees 2-theta. In some embodiments of the present disclosure, the crystalline form of Formula IC is characterized by an XRPD pattern comprising peaks at six or more of 10.5, 11.6, 13.1, 14.2, 14.7, 14.9, 16.2, 17.6, 17.7, 19.6, 28.7, and 28.9 degrees±0.2 degrees 2-theta. In some embodiments of the present disclosure, the crystalline form of Formula IC is characterized by an XRPD pattern comprising peaks at seven or more of 10.5, 11.6, 13.1, 14.2, 14.7, 14.9, 16.2, 17.6, 17.7, 19.6, 28.7, and 28.9 degrees±0.2 degrees 2-theta.

### Formula ID—(Formula I Phosphate Salt)

In some aspects, the disclosure is directed to a crystalline form of the phosphate salt, i.e., Formula ID. In other aspects, the crystalline form of Formula ID is substantially free of any other solid form of Formula ID.

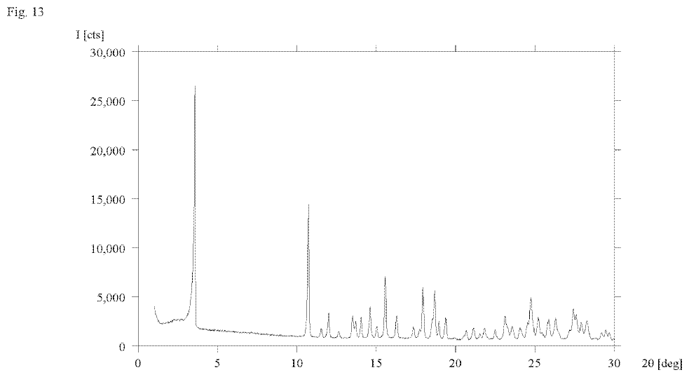

In some embodiments, the crystalline form of Formula ID exhibits an XRPD substantially as shown in FIG. 13. The XRPD of crystalline form of Formula ID shown in FIG. 13 comprises reflection angles (degrees 2-theta±0.2 degrees 2-theta), line spacings (d values), and relative intensities as shown in Table 7:

TABLE 7
XRPD Data for crystalline form of Formula ID
shown in FIG. 13

| Angle | | |
|---|---|---|
| (degrees 2- | | |
| theta ± 0.2 | d Value | Relative |
| degrees 2-theta) | (Å) | Intensity |
| 3.56 | 24.804 | 100 |
| 10.72 | 8.246 | 55 |
| 11.53 | 7.671 | 7 |
| 11.99 | 7.378 | 13 |
| 12.64 | 7.000 | 5 |
| 13.50 | 6.552 | 12 |
| 13.69 | 6.463 | 9 |
| 14.04 | 6.302 | 11 |
| 14.60 | 6.060 | 15 |
| 15.03 | 5.889 | 7 |
| 15.56 | 5.691 | 27 |
| 16.28 | 5.442 | 12 |
| 17.33 | 5.112 | 7 |
| 17.73 | 5.000 | 6 |
| 17.93 | 4.943 | 22 |
| 18.54 | 4.783 | 11 |
| 18.68 | 4.747 | 21 |
| 18.94 | 4.682 | 9 |
| 19.36 | 4.580 | 11 |
| 20.66 | 4.295 | 6 |
| 21.12 | 4.202 | 7 |
| 21.52 | 4.125 | 5 |
| 21.82 | 4.070 | 7 |
| 22.49 | 3.950 | 6 |
| 23.13 | 3.843 | 11 |
| 23.56 | 3.773 | 8 |
| 24.07 | 3.694 | 7 |
| 24.54 | 3.625 | 9 |
| 24.74 | 3.596 | 19 |
| 25.21 | 3.529 | 11 |
| 25.43 | 3.500 | 5 |
| 25.85 | 3.444 | 10 |
| 26.30 | 3.386 | 11 |
| 27.42 | 3.250 | 14 |
| 27.59 | 3.230 | 12 |
| 27.90 | 3.195 | 9 |
| 28.28 | 3.154 | 10 |
| 29.20 | 3.056 | 5 |
| 29.46 | 3.029 | 6 |
| 29.67 | 3.008 | 5 |
| 30.35 | 2.943 | 7 |

In some embodiments of the present disclosure, the crystalline form of Formula ID is characterized by an XRPD pattern comprising a peak at one of the angles listed in Table 7. In other aspects, the crystalline form of Formula ID is characterized by an XRPD pattern comprising more than one peak at one of the angles listed in Table 7 above. In other aspects, the crystalline form of Formula ID is characterized by an XRPD pattern comprising two peaks selected from the angles listed in Table 7 above. In other aspects, the crystalline form of Formula ID is characterized by an XRPD pattern comprising three peaks selected from the angles listed in Table 7 above. In other aspects, the crystalline form of Formula ID is characterized by an XRPD pattern comprising four peaks selected from the angles listed in Table 7 above. In other aspects, the crystalline form of Formula ID is characterized by an XRPD pattern comprising five peaks selected from the angles listed in Table 7 above. In other aspects, the crystalline form of Formula ID is characterized by an XRPD pattern comprising six peaks selected from the angles listed in Table 7 above. In other aspects, the crystalline form of Formula ID is characterized by an XRPD pattern comprising seven peaks selected from the angles listed in Table 7 above. In other aspects, the crystalline form of Formula ID is characterized by an XRPD pattern comprising eight peaks selected from the angles listed in Table 7 above. In other aspects, the crystalline form of Formula ID is characterized by an XRPD pattern comprising nine peaks selected from the angles listed in Table 7 above. In other aspects, the crystalline form of Formula ID is characterized by an XRPD pattern comprising ten peaks selected from the angles listed in Table 7 above. In other aspects, the crystalline form of Formula ID is characterized by an XRPD pattern comprising more than ten peaks selected from the angles listed in Table 7 above.

In some embodiments, the crystalline form of Formula ID is characterized by an XRPD pattern comprising a peak at 3.6 degrees±0.2 degrees 2-theta. In other embodiments, the crystalline form of Formula ID is characterized by an XRPD pattern comprising peaks at 3.6, and 10.7 degrees±0.2 degree 2-theta. In other embodiments, the crystalline form of Formula ID is characterized by an XRPD pattern comprising peaks at 3.6, 10.7, and 15.6 degrees±0.2 degree 2-theta. In yet other embodiments, the crystalline form of Formula ID is characterized by an XRPD pattern comprising peaks at 3.6, 10.7, 15.6, and 17.9 degrees±0.2 degree 2-theta. In yet other embodiments, the crystalline form of Formula ID is characterized by an XRPD pattern comprising peaks at 3.6, 10.7, 15.6, 17.9, and 18.7 degrees±0.2 degree 2-theta.

In some embodiments of the present disclosure, the crystalline form of Formula ID is characterized by an XRPD pattern comprising peaks at two or more of 3.6, 10.7, 15.6, 17.9, and 18.7 degrees±0.2 degrees 2-theta. In some embodiments of the present disclosure, the crystalline form of Formula ID is characterized by an XRPD pattern comprising peaks at three or more of 3.6, 10.7, 15.6, 17.9, and 18.7 degrees±0.2 degrees 2-theta. In some embodiments of the present disclosure, the crystalline form of Formula ID is characterized by an XRPD pattern comprising peaks at four or more of 3.6, 10.7, 15.6, 17.9, and 18.7 degrees±0.2 degrees 2-theta.

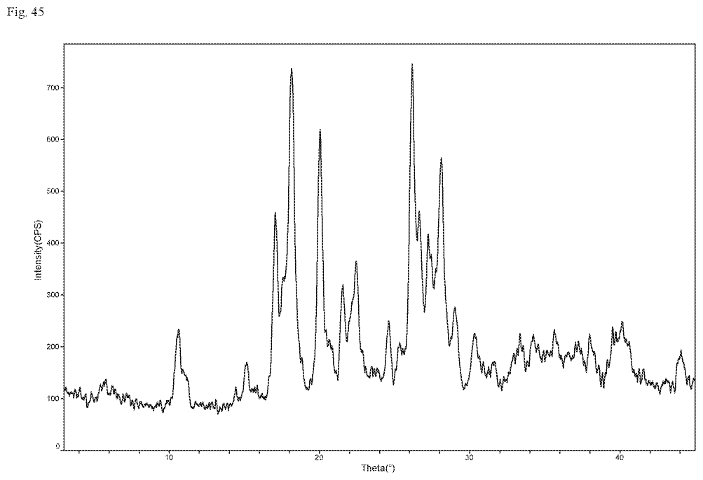

In some embodiments, the crystalline form of Formula ID exhibits an XRPD substantially as shown in FIG. 45. The XRPD of crystalline form of Formula ID shown in FIG. 45 comprises reflection angles (degrees 2-theta±0.2 degrees 2-theta), line spacings (d values), and relative intensities as shown in Table 7A:

TABLE 7A
XRPD Data for crystalline form of Formula ID
shown in FIG. 45

| Angle | | |
|---|---|---|
| (degrees 2- | | |
| theta ± 0.2 | d Value | Relative |
| degrees 2-theta) | (Å) | Intensity |
| 10.64 | 8.3078 | 25.2 |
| 15.14 | 5.847 | 12.4 |
| 17.08 | 5.1871 | 57.8 |
| 18.14 | 4.8863 | 100 |
| 20.04 | 4.4271 | 79.9 |
| 21.54 | 4.122 | 28.3 |
| 22.44 | 3.9587 | 36.2 |
| 24.62 | 3.613 | 18.7 |
| 26.18 | 3.4011 | 98.7 |
| 27.278 | 3.2666 | 32.7 |
| 28.1 | 3.1729 | 72.5 |
| 29.04 | 3.0723 | 16.5 |
| 30.3 | 2.9473 | 16 |
| 33.377 | 2.6824 | 11.1 |
| 34.277 | 2.6139 | 8.3 |
| 37.24 | 2.4125 | 9.5 |
| 38.057 | 2.3625 | 9.5 |
| 39.557 | 2.2763 | 14.7 |
| 40.14 | 2.2446 | 19.5 |
| 44.06 | 2.0536 | 11.6 |
| 44.06 | 2.0536 | 11.6 |

In some embodiments of the present disclosure, the crystalline form of Formula ID is characterized by an XRPD pattern comprising a peak at one of the angles listed in Table 7A. In other aspects, the crystalline form of Formula ID is characterized by an XRPD pattern comprising more than one peak at one of the angles listed in Table 7A above. In other aspects, the crystalline form of Formula ID is characterized by an XRPD pattern comprising two peaks selected from the angles listed in Table 7A above. In other aspects, the crystalline form of Formula ID is characterized by an XRPD pattern comprising three peaks selected from the angles listed in Table 7A above. In other aspects, the crystalline form of Formula ID is characterized by an XRPD pattern comprising four peaks selected from the angles listed in Table 7A above. In other aspects, the crystalline form of Formula ID is characterized by an XRPD pattern comprising five peaks selected from the angles listed in Table 7A above. In other aspects, the crystalline form of Formula ID is characterized by an XRPD pattern comprising six peaks selected from the angles listed in Table 7A above. In other aspects, the crystalline form of Formula ID is characterized by an XRPD pattern comprising seven peaks selected from the angles listed in Table 7A above. In other aspects, the crystalline form of Formula ID is characterized by an XRPD pattern comprising eight peaks selected from the angles listed in Table 7A above. In other aspects, the crystalline form of Formula ID is characterized by an XRPD pattern comprising nine peaks selected from the angles listed in Table 7A above. In other aspects, the crystalline form of Formula ID is characterized by an XRPD pattern comprising ten peaks selected from the angles listed in Table 7A above. In other aspects, the crystalline form of Formula ID is characterized by an XRPD pattern comprising more than ten peaks selected from the angles listed in Table 7A above.

In some embodiments, the crystalline form of Formula ID is characterized by an XRPD pattern comprising a peak at 18.1, 20.0, 26.2, and 28.1 degrees±0.2 degrees 2-theta. In other embodiments, the crystalline form of Formula ID is characterized by an XRPD pattern comprising peaks at 18.1, 20.0, 21.5, 22.4, 26.2, and 28.1 degrees±0.2 degree 2-theta. In other embodiments, the crystalline form of Formula ID is characterized by an XRPD pattern comprising peaks at 17.1, 18.1, 20.0, 26.2, and 28.1 degrees±0.2 degree 2-theta. In yet other embodiments, the crystalline form of Formula ID is characterized by an XRPD pattern comprising peaks at 10.6, 17.1, 18.1, 20.0, 26.2, and 28.1 degrees±0.2 degree 2-theta. In yet other embodiments, the crystalline form of Formula ID is characterized by an XRPD pattern comprising peaks at 10.6, 17.1, 18.1, 20.0, 21.5, 22.4, 26.2, and 28.1 degrees±0.2 degree 2-theta.

In some embodiments of the present disclosure, the crystalline form of Formula ID is characterized by an XRPD pattern comprising peaks at two or more of 10.6, 17.1, 18.1, 20.0, 21.5, 22.4, 26.2, and 28.1 degrees±0.2 degrees 2-theta. In some embodiments of the present disclosure, the crystalline form of Formula ID is characterized by an XRPD pattern comprising peaks at three or more of 10.6, 17.1, 18.1, 20.0, 21.5, 22.4, 26.2, and 28.1 degrees±0.2 degrees 2-theta. In some embodiments of the present disclosure, the crystalline form of Formula ID is characterized by an XRPD pattern comprising peaks at four or more of 10.6, 17.1, 18.1, 20.0, 21.5, 22.4, 26.2, and 28.1 degrees±0.2 degrees 2-theta. In some embodiments of the present disclosure, the crystalline form of Formula ID is characterized by an XRPD pattern comprising peaks at five or more of 10.6, 17.1, 18.1, 20.0, 21.5, 22.4, 26.2, and 28.1 degrees±0.2 degrees 2-theta. In some embodiments of the present disclosure, the crystalline form of Formula ID is characterized by an XRPD pattern comprising peaks at six or more of 10.6, 17.1, 18.1, 20.0, 21.5, 22.4, 26.2, and 28.1 degrees±0.2 degrees 2-theta.

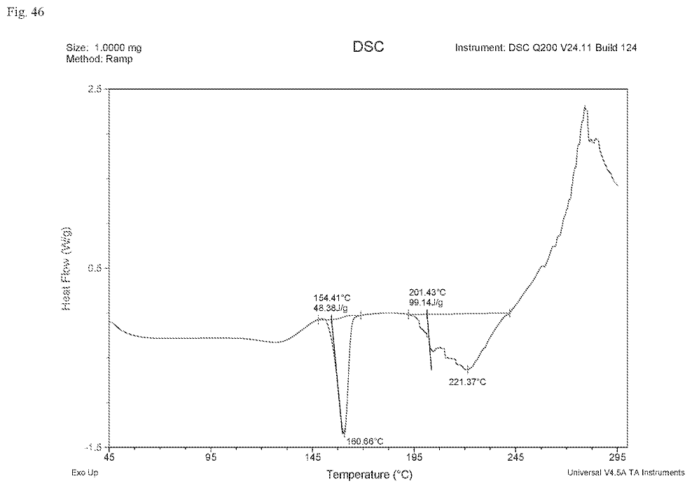

In some embodiments, the crystalline form of Formula ID can be characterized by a DSC thermogram substantially as shown in FIG. 46. As FIG. 46 shows, the crystalline form of Formula ID produced an endothermic peak at 160.66° C. (154.41° C. onset; 48.38 J/g), followed by followed by another endothermic peak at 221.37° C. (201.43° C. onset; 99.14 J/g), when heated at 10° C./min. In some embodiments of the present disclosure, the crystalline form of Formula ID is characterized by a DSC thermogram comprising an endothermic peak at about 161° C. when heated at a rate of 10° C./min. In other embodiments of the present disclosure, the crystalline form of Formula ID is characterized by a DSC thermogram comprising an endothermic peak at about 221° C. when heated at a rate of 10° C./min.

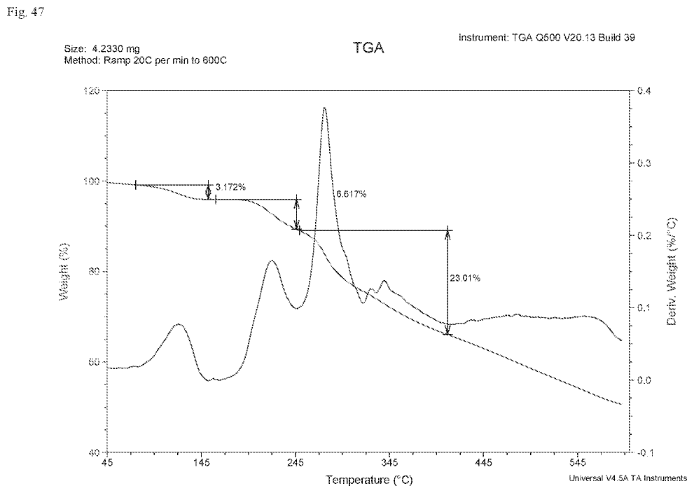

In some embodiments, the crystalline form of Formula ID can be characterized by a TGA profile substantially as shown in FIG. 47 when heated at a rate of 20° C./min. As FIG. 47 shows, the crystalline form of Formula ID lost about 3.2% of its weight upon heating to about 150° C.

### Formula IE—(Formula I Bisulate Salt)

In some aspects, the disclosure is directed to a crystalline form of the bisulfate salt, i.e., Formula IE. In other aspects, the crystalline form of Formula IE is substantially free of any other solid form of Formula IE.

### Formula I—Free Base

In some aspects, the disclosure is directed to crystalline forms of the compound of Formula I:

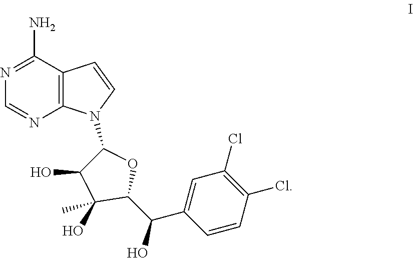 [SMILES: C[C@@]1(O)[C@@H]([C@H](O)c2ccc(Cl)c(Cl)c2)O[C@@H](n2ccc3c(N)ncnc32)[C@@H]1O]

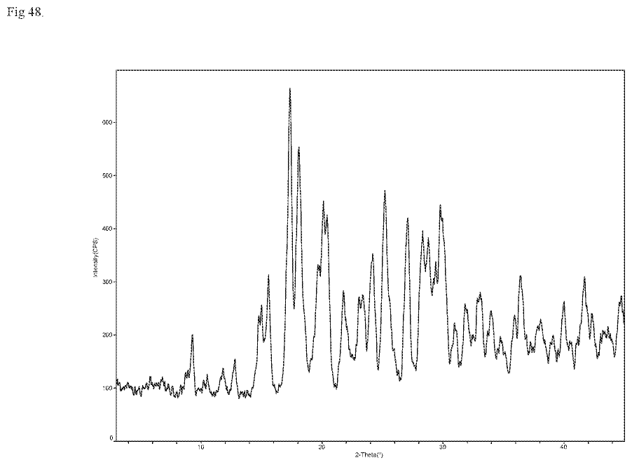

In some embodiments, the crystalline form of Formula I is crystalline Form I (Formula I, Form I). In some embodiments, the crystalline form of Formula I, Form I exhibits an XRPD substantially as shown in FIG. 48. The XRPD of crystalline form of Formula I, Form I shown in FIG. 48 comprises reflection angles (degrees 2-theta±0.2 degrees 2-theta), line spacings (d values), and relative intensities as shown in Table 7B:

TABLE 7B
XRPD Data for crystalline form of Formula I,
Form I shown in FIG. 48

| Angle | | |
|---|---|---|
| (degrees 2- | | |
| theta ± 0.2 | d Value | Relative |
| degrees 2-theta) | (Å) | Intensity |
| 9.299 | 9.5031 | 20.6 |
| 11.82 | 7.4809 | 9.6 |
| 12.8 | 6.9105 | 12.8 |
| 14.981 | 5.9089 | 29.4 |
| 15.58 | 5.683 | 40.4 |
| 17.341 | 5.1097 | 100 |
| 18.1 | 4.8971 | 78.2 |
| 19.66 | 4.5118 | 37.9 |
| 20.42 | 4.3456 | 56.9 |
| 21.818 | 4.0701 | 27.4 |
| 23.341 | 3.808 | 22.9 |
| 24.2 | 3.6747 | 38.4 |
| 25.2 | 3.5311 | 62.1 |
| 27.08 | 3.2901 | 53.1 |
| 28.32 | 3.1487 | 46.8 |
| 28.799 | 3.0974 | 34.5 |
| 29.959 | 2.9801 | 35.4 |
| 30.959 | 2.8861 | 14.3 |
| 31.839 | 2.8083 | 16.8 |
| 33.06 | 2.7073 | 22.4 |
| 33.96 | 2.6376 | 16.8 |
| 34.74 | 2.5801 | 8.8 |
| 35.9 | 2.4994 | 17.3 |
| 36.419 | 2.4649 | 29.1 |
| 38.06 | 2.3624 | 13.7 |
| 39.08 | 2.303 | 9 |
| 39.999 | 2.2522 | 21.8 |
| 41.681 | 2.1651 | 29.4 |
| 42.417 | 2.1292 | 12.8 |

In some embodiments of the present disclosure, the crystalline form of Formula I, Form I is characterized by an XRPD pattern comprising a peak at one of the angles listed in Table 7B. In other aspects, the crystalline form of Formula I, Form I is characterized by an XRPD pattern comprising more than one peak at one of the angles listed in Table 7B above. In other aspects, the crystalline form of Formula I, Form I is characterized by an XRPD pattern comprising two peaks selected from the angles listed in Table 7B above. In other aspects, the crystalline form of Formula I, Form I is characterized by an XRPD pattern comprising three peaks selected from the angles listed in Table 7B above. In other aspects, the crystalline form of Formula I, Form I is characterized by an XRPD pattern comprising four peaks selected from the angles listed in Table 7B above. In other aspects, the crystalline form of Formula I, Form I is characterized by an XRPD pattern comprising five peaks selected from the angles listed in Table 7B above. In other aspects, the crystalline form of Formula I, Form I is characterized by an XRPD pattern comprising six peaks selected from the angles listed in Table 7B above. In other aspects, the crystalline form of Formula I, Form I is characterized by an XRPD pattern comprising seven peaks selected from the angles listed in Table 7B above. In other aspects, the crystalline form of Formula I, Form I is characterized by an XRPD pattern comprising eight peaks selected from the angles listed in Table 7B above. In other aspects, the crystalline form of Formula I, Form I is characterized by an XRPD pattern comprising nine peaks selected from the angles listed in Table 7B above. In other aspects, the crystalline form of Formula I, Form I is characterized by an XRPD pattern comprising ten peaks selected from the angles listed in Table 7B above. In other aspects, the crystalline form of Formula I, Form I is characterized by an XRPD pattern comprising more than ten peaks selected from the angles listed in Table 7B above.

In some embodiments, the crystalline form of Formula I, Form I is characterized by an XRPD pattern comprising a peak at 17.3, and 18.1 degrees±0.2 degrees 2-theta. In other embodiments, the crystalline form of Formula I, Form I is characterized by an XRPD pattern comprising peaks at 17.3, 18.1, 25.2, and 27.1 degrees±0.2 degree 2-theta. In other embodiments, the crystalline form of Formula I, Form I is characterized by an XRPD pattern comprising peaks at 17.3, 18.1, 25.2, 27.1, 28.3, 28.8, and 30.0 degrees±0.2 degree 2-theta. In yet other embodiments, the crystalline form of Formula I, Form I is characterized by an XRPD pattern comprising peaks at 17.3, 18.1, 20.4, 24.2, 25.2, 27.1, 28.3, 28.8, and 30.0 degrees±0.2 degree 2-theta. In yet other embodiments, the crystalline form of Formula I, Form I is characterized by an XRPD pattern comprising peaks at 15.0, 17.3, 18.1, 20.4, 24.2, 25.2, 27.1, 28.3, 28.8, and 30.0 degrees±0.2 degree 2-theta.

In some embodiments of the present disclosure, the crystalline form of Formula I, Form I is characterized by an XRPD pattern comprising peaks at two or more of 15.0, 17.3, 18.1, 20.4, 24.2, 25.2, 27.1, 28.3, 28.8, and 30.0 degrees±0.2 degrees 2-theta. In some embodiments of the present disclosure, the crystalline form of Formula I, Form I is characterized by an XRPD pattern comprising peaks at three or more of 15.0, 17.3, 18.1, 20.4, 24.2, 25.2, 27.1, 28.3, 28.8, and 30.0 degrees±0.2 degrees 2-theta. In some embodiments of the present disclosure, the crystalline form of Formula I, Form I is characterized by an XRPD pattern comprising peaks at four or more of 15.0, 17.3, 18.1, 20.4, 24.2, 25.2, 27.1, 28.3, 28.8, and 30.0 degrees±0.2 degrees 2-theta. In some embodiments of the present disclosure, the crystalline form of Formula I, Form I is characterized by an XRPD pattern comprising peaks at five or more of 15.0, 17.3, 18.1, 20.4, 24.2, 25.2, 27.1, 28.3, 28.8, and 30.0 degrees±0.2 degrees 2-theta. In some embodiments of the present disclosure, the crystalline form of Formula I, Form I is characterized by an XRPD pattern comprising peaks at six or more of 15.0, 17.3, 18.1, 20.4, 24.2, 25.2, 27.1, 28.3, 28.8, and 30.0 degrees±0.2 degrees 2-theta.

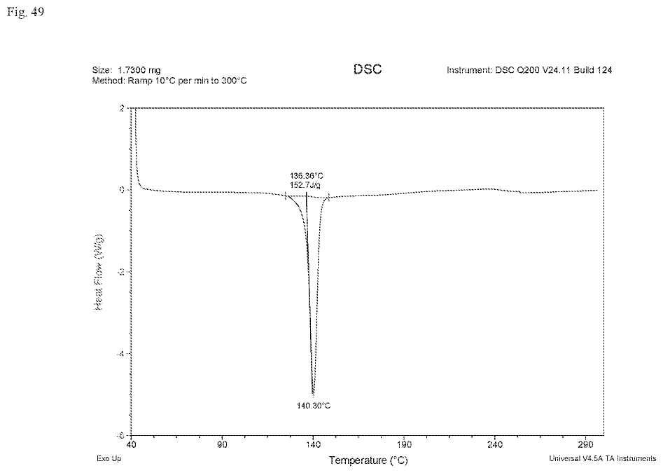

In some embodiments, the crystalline form of Formula I, Form I can be characterized by a DSC thermogram substantially as shown in FIG. 49. As FIG. 49 shows, the crystalline form of Formula I, Form I produced an endothermic peak at 140.30° C. (136.36° C. onset; 152.7 J/g) when heated at 10° C./min. In some embodiments of the present disclosure, the crystalline form of Formula I, Form I is characterized by a DSC thermogram comprising an endothermic peak at about 140° C. when heated at a rate of 10° C./min.

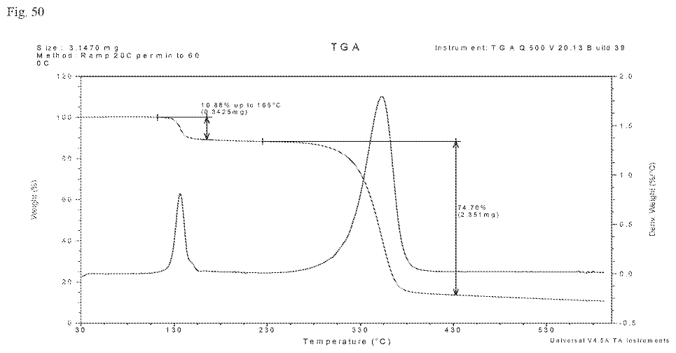

In some embodiments, Formula I, Form I can be characterized by a TGA profile substantially as shown in FIG. 50 when heated at a rate of 20° C./min. As FIG. 50 shows, the crystalline form of Formula I, Form I lost about 10.9% of its weight upon heating to about 150° C.

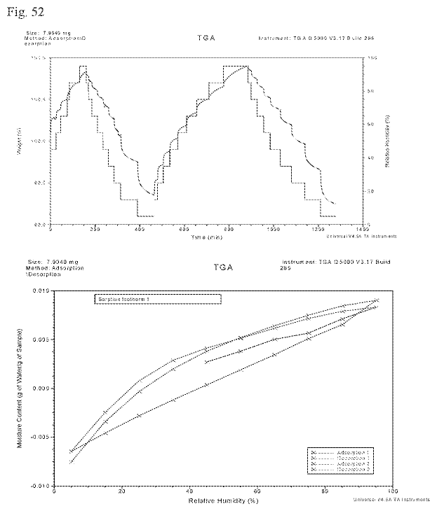

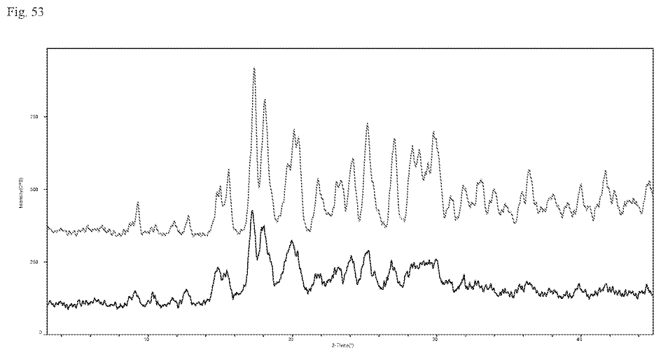

In some embodiments, Formula I, Form I can be characterized by a DVS profile substantially as shown in FIG. 52. As shown in FIG. 53, DVS did not change the polymorphic form.

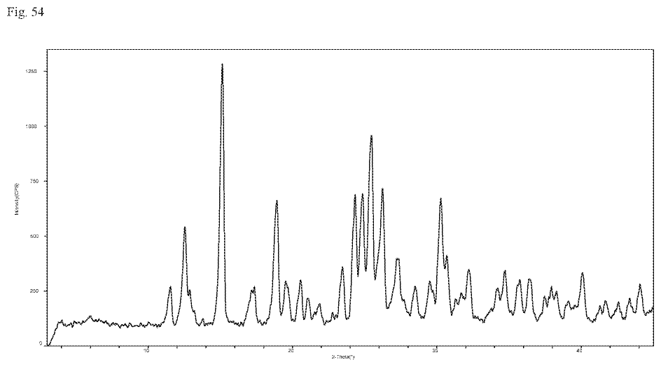

In some embodiments, the crystalline form of Formula I is crystalline Form II (Formula I, Form II). In some embodiments, the crystalline form of Formula I, Form II exhibits an XRPD substantially as shown in FIG. 54. The XRPD of crystalline form of Formula I, Form II shown in FIG. 54 comprises reflection angles (degrees 2-theta±0.2 degrees 2-theta), line spacings (d values), and relative intensities as shown in Table 7C:

TABLE 7C
XRPD Data for crystalline form of Formula I,
Form II shown in FIG. 54

| Angle | | |
|---|---|---|
| (degrees 2- | | |
| theta ± 0.2 | d Value | Relative |
| degrees 2-theta) | (Å) | Intensity |
| 4.039 | 21.8576 | 9.6 |
| 11.54 | 7.6618 | 15 |
| 12.54 | 7.0528 | 37.5 |
| 15.14 | 5.8471 | 100 |
| 17.36 | 5.1042 | 15 |
| 18.92 | 4.6866 | 46.9 |
| 19.54 | 4.5393 | 15.6 |
| 20.56 | 4.3163 | 15.1 |
| 21.099 | 4.2072 | 9.2 |
| 21.898 | 4.0554 | 7.4 |
| 23.46 | 3.7889 | 20.3 |
| 24.34 | 3.6539 | 47.4 |
| 24.86 | 3.5786 | 47.8 |
| 25.46 | 3.4956 | 67.3 |
| 26.24 | 3.3934 | 41.2 |
| 27.221 | 3.2734 | 20.3 |
| 28.481 | 3.1313 | 11.6 |
| 29.501 | 3.0254 | 13.6 |
| 30.28 | 2.9493 | 44.9 |
| 32.22 | 2.776 | 19 |
| 34.22 | 2.6182 | 11.9 |
| 34.739 | 2.5802 | 17.8 |
| 35.74 | 2.5102 | 14.1 |
| 36.42 | 2.4649 | 15.3 |
| 37.46 | 2.3988 | 9.2 |
| 38.299 | 2.3482 | 10.7 |
| 39.12 | 2.3008 | 6.9 |
| 40.08 | 2.2478 | 18.4 |
| 41.661 | 2.1661 | 7.7 |
| 42.58 | 2.1215 | 6.4 |
| 43.36 | 2.0851 | 7.5 |
| 44.079 | 2.0527 | 12.1 |

In some embodiments of the present disclosure, the crystalline form of Formula I, Form II is characterized by an XRPD pattern comprising a peak at one of the angles listed in Table 7C. In other aspects, the crystalline form of Formula I, Form II is characterized by an XRPD pattern comprising more than one peak at one of the angles listed in Table 7C above. In other aspects, the crystalline form of Formula I, Form II is characterized by an XRPD pattern comprising two peaks selected from the angles listed in Table 7C above. In other aspects, the crystalline form of Formula I, Form II is characterized by an XRPD pattern comprising three peaks selected from the angles listed in Table 7C above. In other aspects, the crystalline form of Formula I, Form II is characterized by an XRPD pattern comprising four peaks selected from the angles listed in Table 7C above. In other aspects, the crystalline form of Formula I, Form II is characterized by an XRPD pattern comprising five peaks selected from the angles listed in Table 7C above. In other aspects, the crystalline form of Formula I, Form II is characterized by an XRPD pattern comprising six peaks selected from the angles listed in Table 7C above. In other aspects, the crystalline form of Formula I, Form II is characterized by an XRPD pattern comprising seven peaks selected from the angles listed in Table 7C above. In other aspects, the crystalline form of Formula I, Form II is characterized by an XRPD pattern comprising eight peaks selected from the angles listed in Table 7C above. In other aspects, the crystalline form of Formula I, Form II is characterized by an XRPD pattern comprising nine peaks selected from the angles listed in Table 7C above. In other aspects, the crystalline form of Formula I, Form II is characterized by an XRPD pattern comprising ten peaks selected from the angles listed in Table 7C above. In other aspects, the crystalline form of Formula I, Form II is characterized by an XRPD pattern comprising more than ten peaks selected from the angles listed in Table 7C above.

In some embodiments, the crystalline form of Formula I, Form II is characterized by an XRPD pattern comprising a peak at 23.5 and 24.9 degrees±0.2 degrees 2-theta. In other embodiments, the crystalline form of Formula I, Form II is characterized by an XRPD pattern comprising peaks at 18.9, 23.5, 24.3, and 24.9, degrees±0.2 degree 2-theta. In other embodiments, the crystalline form of Formula I, Form II is characterized by an XRPD pattern comprising peaks at 17.4, 18.9, 23.5, 24.3, and 24.9, 25.5, and 30.3 degrees±0.2 degree 2-theta. In other embodiments, the crystalline form of Formula I, Form II is characterized by an XRPD pattern comprising peaks at 15.1, 17.4, 18.9, 23.5, 24.3, and 24.9 degrees±0.2 degree 2-theta. In yet other embodiments, the crystalline form of Formula I, Form II is characterized by an XRPD pattern comprising peaks at 15.1, 17.4, 18.9, 23.5, 24.3, 24.9, and 25.5 degrees±0.2 degree 2-theta. In yet other embodiments, the crystalline form of Formula I, Form II is characterized by an XRPD pattern comprising peaks at 15.1, 17.4, 18.9, 23.5, 24.3, 24.9, 25.5, and 30.3 degrees±0.2 degree 2-theta.

In some embodiments of the present disclosure, the crystalline form of Formula I, Form II is characterized by an XRPD pattern comprising peaks at two or more of 15.1, 17.4, 18.9, 23.5, 24.3, 24.9, 25.5, and 30.3 degrees±0.2 degrees 2-theta. In some embodiments of the present disclosure, the crystalline form of Formula I, Form II is characterized by an XRPD pattern comprising peaks at three or more of 15.1, 17.4, 18.9, 23.5, 24.3, 24.9, 25.5, and 30.3 degrees±0.2 degrees 2-theta. In some embodiments of the present disclosure, the crystalline form of Formula I, Form II is characterized by an XRPD pattern comprising peaks at four or more of 15.1, 17.4, 18.9, 23.5, 24.3, 24.9, 25.5, and 30.3 degrees±0.2 degrees 2-theta. In some embodiments of the present disclosure, the crystalline form of Formula I, Form II is characterized by an XRPD pattern comprising peaks at five or more of 15.1, 17.4, 18.9, 23.5, 24.3, 24.9, 25.5, and 30.3 degrees±0.2 degrees 2-theta. In some embodiments of the present disclosure, the crystalline form of Formula I, Form II is characterized by an XRPD pattern comprising peaks at six or more of 15.1, 17.4, 18.9, 23.5, 24.3, 24.9, 25.5, and 30.3 degrees±0.2 degrees 2-theta.

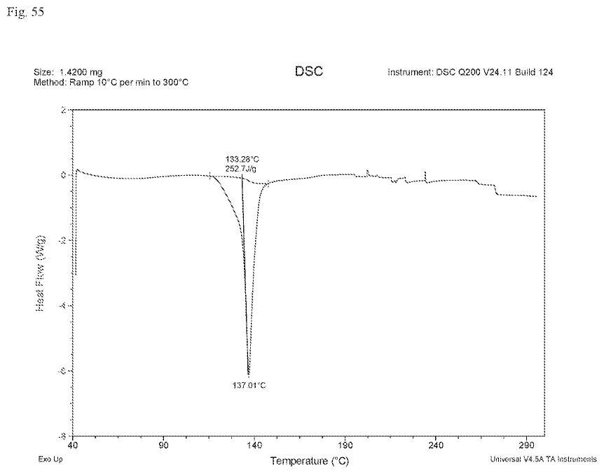

In some embodiments, the crystalline form of Formula I, Form II can be characterized by a DSC thermogram substantially as shown in FIG. 55. As FIG. 55 shows, the crystalline form of Formula I, Form II produced an endothermic peak at 137.01° C. (133.28° C. onset; 252.7 J/g) when heated at 10° C./min. In some embodiments of the present disclosure, the crystalline form of Formula I, Form II is characterized by a DSC thermogram comprising an endothermic peak at about 137° C. when heated at a rate of 10° C./min.

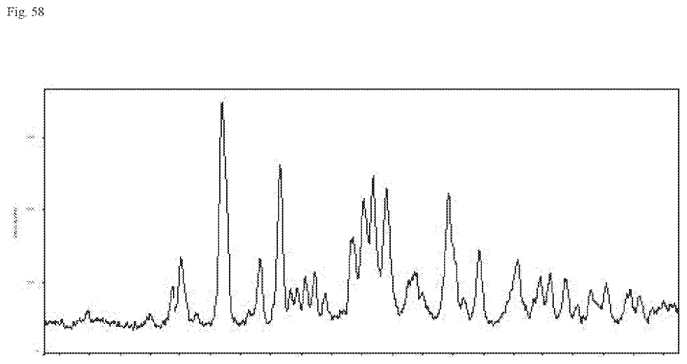

In some embodiments, the crystalline form of Formula I, Form II exhibits an XRPD substantially as shown in FIG. 58.

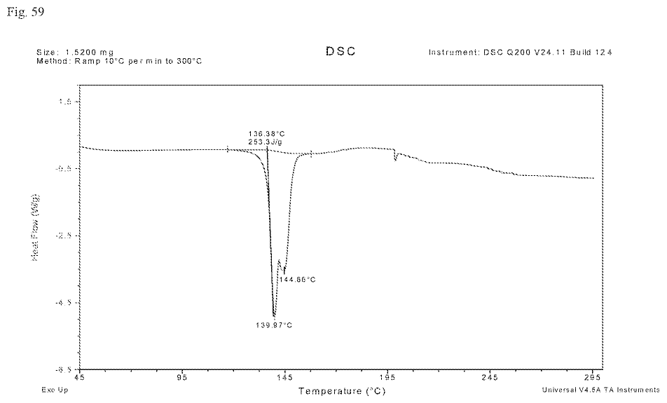

In some embodiments, the crystalline form of Formula I, Form II exhibits a DSC thermogram substantially as shown in FIG. 59.

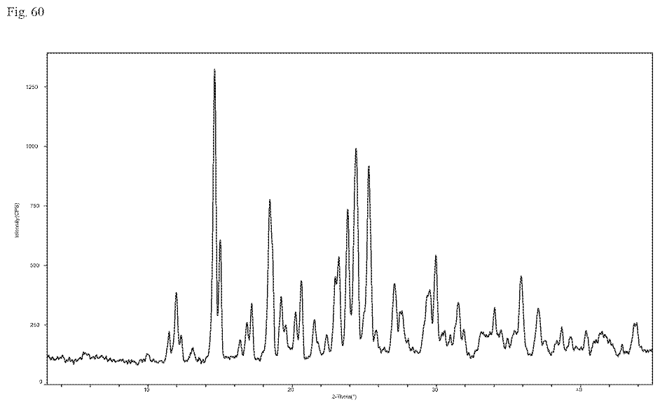

In some embodiments, the crystalline form of Formula I, Form II exhibits an XRPD substantially as shown in FIG. 60.

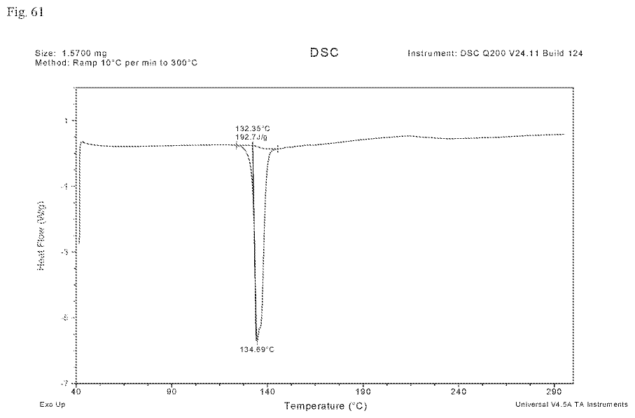

In some embodiments, the crystalline form of Formula I, Form II exhibits a DSC thermogram substantially as shown in FIG. 61.

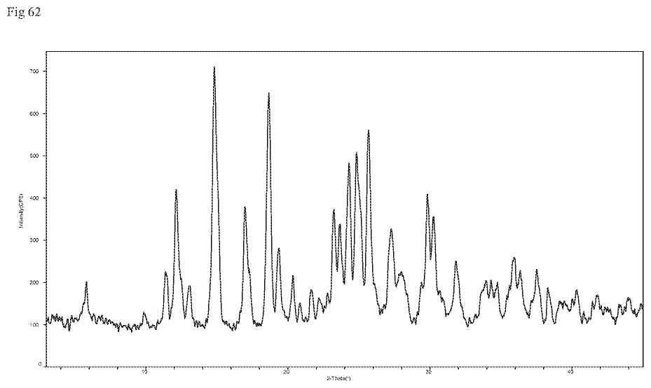

In some embodiments, the crystalline form of Formula I, Form II exhibits an XRPD substantially as shown in FIG. 62.

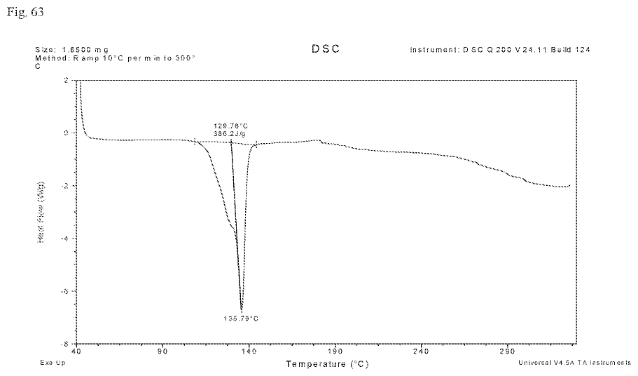

In some embodiments, the crystalline form of Formula I, Form II exhibits a DSC thermogram substantially as shown in FIG. 63.

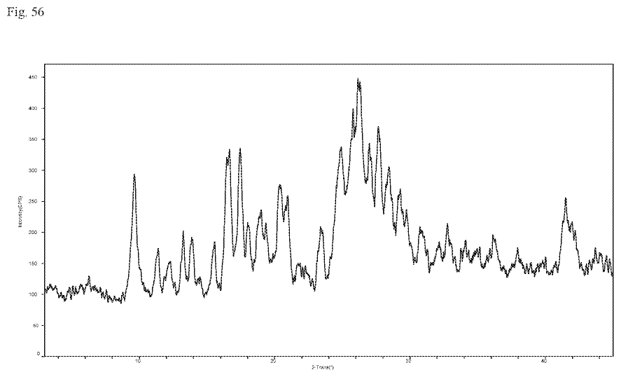

In some embodiments, the crystalline form of Formula I is crystalline Form III (Formula I, Form III). In some embodiments, the crystalline form of Formula I, Form III exhibits an XRPD substantially as shown in FIG. 56. The XRPD of crystalline form of Formula I, Form III shown in FIG. 56 comprises reflection angles (degrees 2-theta±0.2 degrees 2-theta), line spacings (d values), and relative intensities as shown in Table 7D:

TABLE 7D
XRPD Data for crystalline form of Formula I,
Form III shown in FIG. 56

| Angle | | |
|---|---|---|
| (degrees 2- | | |
| theta ± 0.2 | d Value | Relative |
| degrees 2-theta) | (Å) | Intensity |
| 6.263 | 14.1012 | 18.5 |
| 9.621 | 9.1857 | 96.2 |
| 11.399 | 7.756 | 36.5 |
| 12.299 | 7.1907 | 25.6 |
| 13.24 | 6.6814 | 49.3 |
| 13.861 | 6.3836 | 45 |
| 15.56 | 5.6902 | 38.9 |
| 16.64 | 5.3231 | 95.7 |
| 17.44 | 5.0808 | 99.5 |
| 18.1 | 4.897 | 27.5 |
| 19 | 4.6671 | 42.7 |
| 20.38 | 4.354 | 65.9 |
| 20.96 | 4.2348 | 62.6 |
| 23.381 | 3.8014 | 44.1 |
| 24.92 | 3.5702 | 100 |
| 25.78 | 3.4529 | 86.3 |
| 26.3 | 3.3858 | 89.6 |
| 27 | 3.2997 | 33.2 |
| 27.66 | 3.2223 | 63 |
| 28.44 | 3.1357 | 39.3 |
| 29.28 | 3.0477 | 38.9 |
| 30.86 | 2.8951 | 28.4 |
| 32.142 | 2.7825 | 23.2 |
| 32.94 | 2.7169 | 24.2 |
| 36.299 | 2.4729 | 25.1 |
| 41.519 | 2.1732 | 58.8 |
| 42.2 | 2.1397 | 33.2 |

In some embodiments of the present disclosure, the crystalline form of Formula I, Form III is characterized by an XRPD pattern comprising a peak at one of the angles listed in Table 7D. In other aspects, the crystalline form of Formula I, Form III is characterized by an XRPD pattern comprising more than one peak at one of the angles listed in Table 7D above. In other aspects, the crystalline form of Formula I, Form III is characterized by an XRPD pattern comprising two peaks selected from the angles listed in Table 7D above. In other aspects, the crystalline form of Formula I, Form III is characterized by an XRPD pattern comprising three peaks selected from the angles listed in Table 7D above. In other aspects, the crystalline form of Formula I, Form III is characterized by an XRPD pattern comprising four peaks selected from the angles listed in Table 7D above. In other aspects, the crystalline form of Formula I, Form III is characterized by an XRPD pattern comprising five peaks selected from the angles listed in Table 7D above. In other aspects, the crystalline form of Formula I, Form III is characterized by an XRPD pattern comprising six peaks selected from the angles listed in Table 7D above. In other aspects, the crystalline form of Formula I, Form III is characterized by an XRPD pattern comprising seven peaks selected from the angles listed in Table 7D above. In other aspects, the crystalline form of Formula I, Form III is characterized by an XRPD pattern comprising eight peaks selected from the angles listed in Table 7D above. In other aspects, the crystalline form of Formula I, Form III is characterized by an XRPD pattern comprising nine peaks selected from the angles listed in Table 7D above. In other aspects, the crystalline form of Formula I, Form III is characterized by an XRPD pattern comprising ten peaks selected from the angles listed in Table 7D above. In other aspects, the crystalline form of Formula I, Form III is characterized by an XRPD pattern comprising more than ten peaks selected from the angles listed in Table 7D above.

In some embodiments, the crystalline form of Formula I, Form III is characterized by an XRPD pattern comprising a peak at 16.6, and 17.4 degrees±0.2 degrees 2-theta. In other embodiments, the crystalline form of Formula I, Form III is characterized by an XRPD pattern comprising peaks at 17.4, 20.4, and 25.8 degrees±0.2 degree 2-theta. In other embodiments, the crystalline form of Formula I, Form III is characterized by an XRPD pattern comprising peaks at 17.4, 20.4, 24.9, 25.8, and 26.3 degrees±0.2 degree 2-theta. In yet other embodiments, the crystalline form of Formula I, Form III is characterized by an XRPD pattern comprising peaks at 16.6, 17.4, 20.4, 24.9, 25.8, 26.3, and 27.7 degrees±0.2 degree 2-theta. In yet other embodiments, the crystalline form of Formula I, Form III is characterized by an XRPD pattern comprising peaks at 9.2, 16.6, 17.4, 20.4, 24.9, 25.8, 26.3, 27.7, and 41.5 degrees±0.2 degree 2-theta.

In some embodiments of the present disclosure, the crystalline form of Formula I, Form III is characterized by an XRPD pattern comprising peaks at two or more of 9.2, 16.6, 17.4, 20.4, 24.9, 25.8, 26.3, 27.7, and 41.5 degrees±0.2 degrees 2-theta. In some embodiments of the present disclosure, the crystalline form of Formula I, Form III is characterized by an XRPD pattern comprising peaks at three or more of 9.2, 16.6, 17.4, 20.4, 24.9, 25.8, 26.3, 27.7, and 41.5 degrees±0.2 degrees 2-theta. In some embodiments of the present disclosure, the crystalline form of Formula I, Form III is characterized by an XRPD pattern comprising peaks at four or more of 9.2, 16.6, 17.4, 20.4, 24.9, 25.8, 26.3, 27.7, and 41.5 degrees±0.2 degrees 2-theta. In some embodiments of the present disclosure, the crystalline form of Formula I, Form III is characterized by an XRPD pattern comprising peaks at five or more of 9.2, 16.6, 17.4, 20.4, 24.9, 25.8, 26.3, 27.7, and 41.5 degrees±0.2 degrees 2-theta. In some embodiments of the present disclosure, the crystalline form of Formula I, Form III is characterized by an XRPD pattern comprising peaks at six or more of 9.2, 16.6, 17.4, 20.4, 24.9, 25.8, 26.3, 27.7, and 41.5 degrees±0.2 degrees 2-theta.

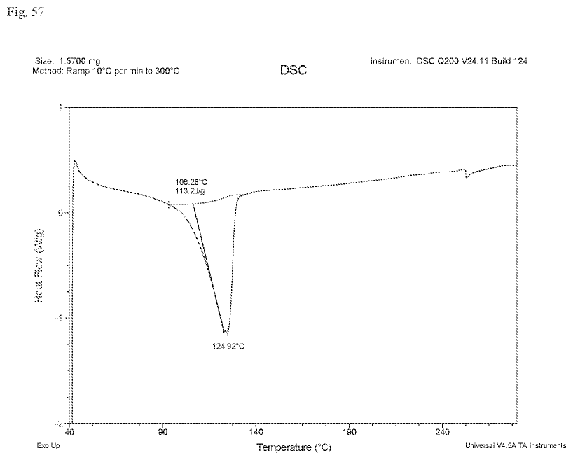

In some embodiments, the crystalline form of Formula I, Form III can be characterized by a DSC thermogram substantially as shown in FIG. 57. As FIG. 57 shows, the crystalline form of Formula I, Form III produced an endothermic peak at 124.92° C. (106.28° C. onset; 113.2 J/g) when heated at 10° C./min. In some embodiments of the present disclosure, the crystalline form of Formula I, Form III is characterized by a DSC thermogram comprising an endothermic peak at about 125° C. when heated at a rate of 10° C./min.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof polyoxyethylated vitamins and derivatives thereof; polyoxyethylene-polyoxypropylene block copolymers; and mixtures thereof polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phytosterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25% o, 50%), 100% o, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

In some embodiments, the pharmaceutical composition comprises a compound of formula IA, mannitol, microcrystalline cellulose, crospovidone, and magnesium stearate.

In some embodiments, the pharmaceutical composition comprises a compound of formula IB, mannitol, microcrystalline cellulose, crospovidone, and magnesium stearate.

In some embodiments, the pharmaceutical composition comprises a compound of formula IC, mannitol, microcrystalline cellulose, crospovidone, and magnesium stearate.

In some embodiments, the pharmaceutical composition comprises a compound of formula ID, mannitol, microcrystalline cellulose, crospovidone, and magnesium stearate.

In some embodiments, the pharmaceutical composition comprises a compound of formula IE, mannitol, microcrystalline cellulose, crospovidone, and magnesium stearate.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

Pharmaceutical Compositions for Topical (e.g. Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g. by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC50” refers to the half maximal inhibitory concentration of an inhibitor in inhibiting biological or biochemical function. This quantitative measure indicates how much of a particular inhibitor is needed to inhibit a given biological process (or component of a process, i.e. an enzyme, cell, cell receptor or microorganism) by half. In other words, it is the half maximal (50%) inhibitory concentration (IC) of a substance (50% IC, or IC50). EC50 refers to the plasma concentration required for obtaining 50%> of a maximum effect in vivo.

In some embodiments, the subject methods utilize a PRMT5 inhibitor with an IC50 value of about or less than a predetermined value, as ascertained in an in vitro assay. In some embodiments, the PRMT5 inhibitor inhibits PRMT5 a with an IC50 value of about 1 nM or less, 2 nM or less, 5 nM or less, 7 nM or less, 10 nM or less, 20 nM or less, 30 nM or less, 40 nM or less, 50 nM or less, 60 nM or less, 70 nM or less, 80 nM or less, 90 nM or less, 100 nM or less, 120 nM or less, 140 nM or less, 150 nM or less, 160 nM or less, 170 nM or less, 180 nM or less, 190 nM or less, 200 nM or less, 225 nM or less, 250 nM or less, 275 nM or less, 300 nM or less, 325 nM or less, 350 nM or less, 375 nM or less, 400 nM or less, 425 nM or less, 450 nM or less, 475 nM or less, 500 nM or less, 550 nM or less, 600 nM or less, 650 nM or less, 700 nM or less, 750 nM or less, 800 nM or less, 850 nM or less, 900 nM or less, 950 nM or less, 1 μM or less, 1.1 μM or less, 1.2 μM or less, 1.3 μM or less, 1.4 μM or less, 1.5 μM or less, 1.6 μM or less, 1.7 μM or less, 1.8 μM or less, 1.9 μM or less, 2 μM or less, 5 μM or less, 10 μM or less, 15 μM or less, 20 μM or less, 25 μM or less, 30 μM or less, 40 μM or less, 50 μM, 60 μM, 70 μM, 80 μM, 90 μM, 100 μM, 200 μM, 300 μM, 400 μM, or 500 μM, or less, (or a number in the range defined by and including any two numbers above).

In some embodiments, the PRMT5 inhibitor selectively inhibits PRMT5 a with an IC50 value that is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two, or three other PRMTs.

In some embodiments, the PRMT5 inhibitor selectively inhibits PRMT5 a with an IC50 value that is less than about 1 nM, 2 nM, 5 nM, 7 nM, 10 nM, 20 nM, 30 nM, 40 nM, 50 nM, 60 nM, 70 nM, 80 nM, 90 nM, 100 nM, 120 nM, 140 nM, 150 nM, 160 nM, 170 nM, 180 nM, 190 nM, 200 nM, 225 nM, 250 nM, 275 nM, 300 nM, 325 nM, 350 nM, 375 nM, 400 nM, 425 nM, 450 nM, 475 nM, 500 nM, 550 nM, 600 nM, 650 nM, 700 nM, 750 nM, 800 nM, 850 nM, 900 nM, 950 nM, 1 μM, 1.1 μM, 1.2 μM, 1.3 μM, 1.4 μM, 1.5 μM, 1.6 μM, 1.7 μM, 1.8 μM, 1.9 μM, 2 μM, 5 μM, 10 μM, 15 μM, 20 μM, 25 μM, 30 μM, 40 μM, 50 μM, 60 μM, 70 μM, 80 μM, 90 μM, 100 μM, 200 μM, 300 μM, 400 μM, or 500 μM (or in the range defined by and including any two numbers above), and said IC50 value is at least 2, 3, 4, 5, 6, 7, 8, 9, 10, 15, 20, 25, 30, 35, 40, 45, 50, 100, or 1000 times less (or a number in the range defined by and including any two numbers above) than its IC50 value against one, two or three other PRMTs.

The subject methods are useful for treating a disease condition associated with PRMT5. Any disease condition that results directly or indirectly from an abnormal activity or expression level of PRMT5 can be an intended disease condition.

Different disease conditions associated with PRMT5 have been reported. PRMT5 has been implicated, for example, in a variety of human cancers as well as a number of hemoglobinopathies.

Non-limiting examples of such conditions include but are not limited to Acanthoma, Acinic cell carcinoma, Acoustic neuroma, Acral lentiginous melanoma, Acrospiroma, Acute eosinophilic leukemia, Acute lymphoblastic leukemia, Acute lymphocytic leukemia, Acute megakaryoblastic leukemia, Acute monocytic leukemia, Acute myeloblasts leukemia with maturation, Acute myeloid dendritic cell leukemia, Acute myeloid leukemia, Acute myelogenous leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epidermoid cancer, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Head and Neck Cancer, Head and neck cancer, Heart cancer, Hemoglobinopathies such as b-thalassemia and sickle cell disease (SCD), Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mastocytosis, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplasia Disease, Myelodysplasia Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene onChromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In some embodiments, said method is for treating a disease selected from breast cancer, lung cancer, pancreatic cancer, prostate cancer, colon cancer, ovarian cancer, uterine cancer, cervical cancer, leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS), epidermoid cancer, or hemoglobinopathies such as b-thalassemia and sickle cell disease (SCD).

In other embodiments, said method is for treating a disease selected from breast cancer, lung cancer, pancreatic cancer, prostate cancer, colon cancer, ovarian cancer, uterine cancer, or cervical cancer.

In other embodiments, said method is for treating a disease selected from leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS), epidermoid cancer, or hemoglobinopathies such as b-thalassemia and sickle cell disease (SCD).

In yet other embodiments, said method is for treating a disease selected from CDKN2A deleted cancers; 9P deleted cancers; MTAP deleted cancers; glioblastoma, NSCLC, head and neck cancer, bladder cancer, or hepatocellular carcinoma.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

In other aspects, compounds of the disclosure, as well as pharmaceutical compositions comprising them, can be administered to treat any of the described diseases, alone or in combination with one or more other chemotherapeutic agents. Examples of other chemotherapeutic agents include, for example, abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat, and zoledronate, as well as any combination thereof.

In other aspects, the other agent is a therapeutic agent that targets an epigenetic regulator. Examples of epigenetic regulator agents include, for example, bromodomain inhibitors, the histone lysine methyltransferases, histone arginine methyl transferases, histone demethylases, histone deacetylases, histone acetylases, and DNA methyltransferases, as well as any combination thereof. Histone deacetylase inhibitors are preferred in some aspects, and include, for example, vorinostat.

In other methods wherein the disease to be treated is cancer or another proliferative disease, the compounds of the disclosure, as well as pharmaceutical compositions comprising them, can be administered in combination with targeted therapy agents. Targeted therapies include, for example, JAK kinase inhibitors (e.g. Ruxolitinib), PI3 kinase inhibitors (including PI3K-delta selective and broad spectrum PI3K inhibitors), MEK inhibitors, Cyclin Dependent kinase inhibitors (e.g, CDK4/6 inhibitors), BRAF inhibitors, mTOR inhibitors, proteasome inhibitors (e.g., Bortezomib, Carfilzomib), HDAC-inhibitors (e.g., panobinostat, vorinostat), DNA methyl transferase inhibitors, dexamethasone, bromo and extra terminal family members, BTK inhibitors (e.g., ibrutinib, acalabrutinib), BCL2 inhibitors (e.g., venetoclax), MCL1 inhibitors, PARP inhibitors, FLT3 inhibitors, and LSD1 inhibitors, as well as any combination thereof.

In other methods wherein the disease to be treated is cancer or another proliferative disease, the compounds of the disclosure, as well as pharmaceutical compositions comprising them, can be administered in combination with an immune checkpoint inhibitor agents. Immune checkpoint inhibitors include, for example, inhibitors of PD-1, for example, an anti-PD-1 monoclonal antibody. Examples of anti-PD-1 monoclonal antibodies include, for example, nivolumab, pembrolizumab (also known as MK-3475), pidilizumab, SHR-1210, PDR001, and AMP-224, as well as combinations thereof. In some aspects, the anti-PD1 antibody is nivolumab. In some aspects, the anti-PD1 antibody is pembrolizumab. In some aspects, the immunce checkpoint inhibitor is an inhibitor of PD-L1, for example, an anti-PD-L1 monoclonal antibody. In some aspects, the anti-PD-L1 monoclonal antibody is BMS-935559, MEDI4736, MPDL3280A (also known as RG7446), or MSB0010718C, or any combination thereof. In some aspects, the anti-PD-L1 monoclonal antibody is MPDL3280A or MEDI4736. In other aspects, the immune checkpoint inhibitor is an inhibitor of CTLA-4, for example, and anti-CTLA-4 antibody. In some aspects, the anti-CTLA-4 antibody is ipilimumab.

In other methods wherein the disease to be treated is cancer or another proliferative disease, the compounds of the disclosure, as well as pharmaceutical compositions comprising them, can be administered in combination with an alkylating agent (e.g., cyclophosphamide (CY), melphalan (MEL), and bendamustine), a proteasome inhibitor agent (e.g., carfilzomib), a corticosteroid agent (e.g., dexamethasone (DEX)), or an immunomodulatory agent (e.g., lenalidomide (LEN) or pomalidomide (POM)), or any combination thereof.

In some embodiments, the disease to be treated is an autoimmune condition or an inflammatory condition. In these aspects, the compounds of the disclosure, as well as pharmaceutical compositions comprising them, can be administered in combination with a corticosteroid agent such as, for example, triamcinolone, dexamethasone, fluocinolone, cortisone, prednisolone, or flumetholone, or any combination thereof.

In other methods wherein the disease to be treated is an autoimmune condition or an inflammatory condition, the compounds of the disclosure, as well as pharmaceutical compositions comprising them, can be administered in combination with an immune suppressant agent such as, for example, fluocinolone acetonide (RETISERT™), rimexolone (AL-2178, VEXOL™ ALCO™) or cyclosporine (RESTASIS™), or any combination thereof.

In some embodiments, the disease to be treated is beta-thalassemia or sickle cell disease. In these aspects, the compounds of the disclosure, as well as pharmaceutical compositions comprising them, can be administered in combination with one or more agents such as, for example, HYDREA™ (hydroxyurea).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

The compound of Formula I, and pharmaceutically acceptable salts thereof, can be prepared, for example, by reference to the following schemes and procedures.

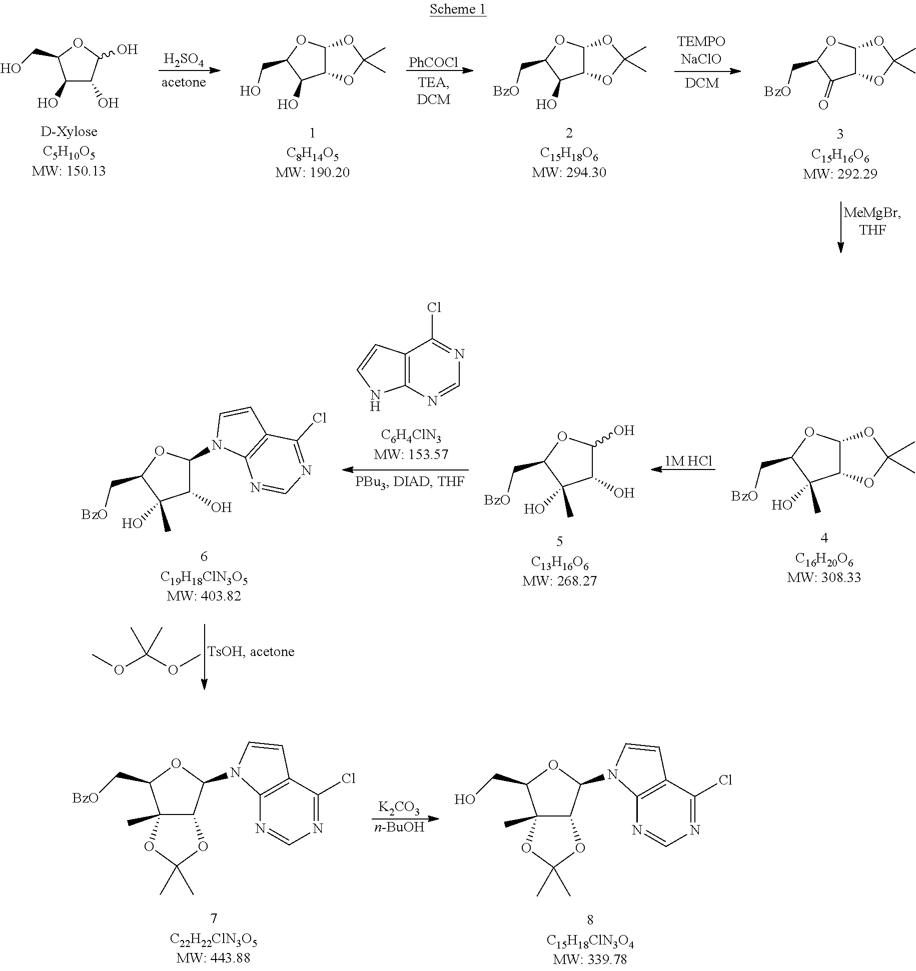

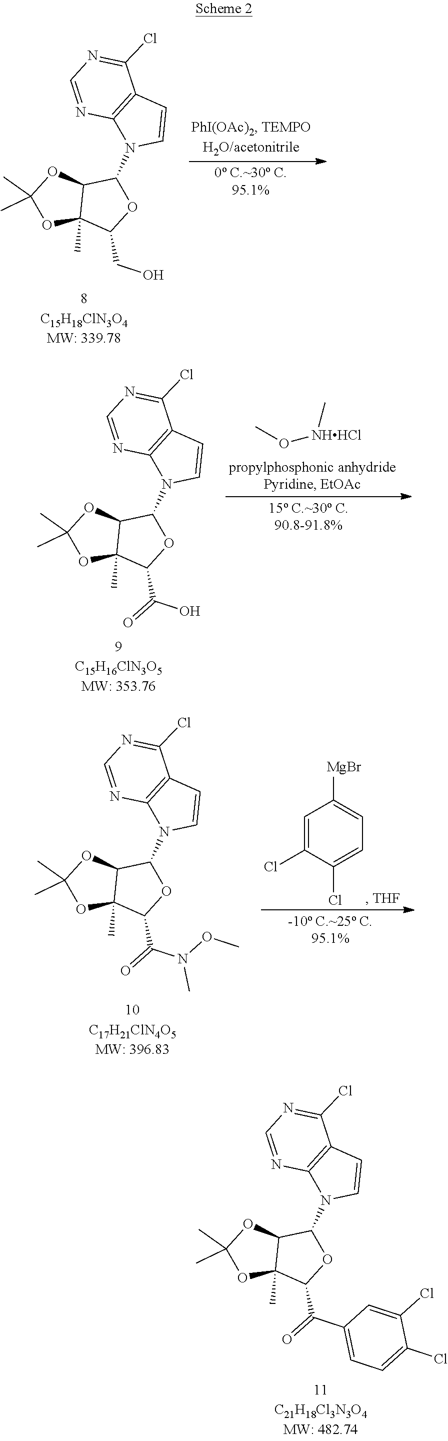

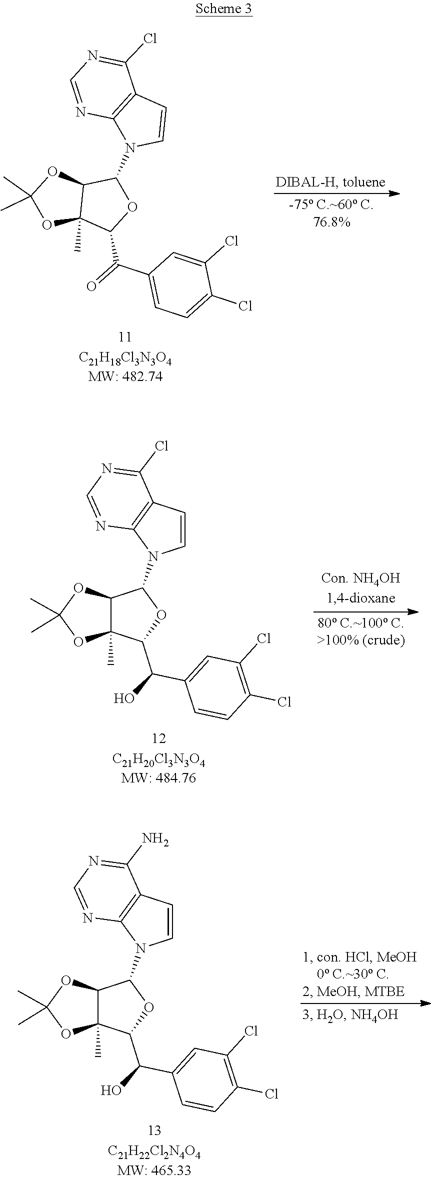 [SMILES: CC1(C)O[C@H]2[C@H](n3ccc4c(Cl)ncnc43)O[C@H](C(=O)c3ccc(Cl)c(Cl)c3)[C@@]2(C)O1 ; CC1(C)O[C@H]2[C@H](n3ccc4c(Cl)ncnc43)O[C@H]([C@H](O)c3ccc(Cl)c(Cl)c3)[C@@]2(C)O1 ; CC1(C)O[C@H]2[C@H](n3ccc4c(N)ncnc43)O[C@H]([C@H](O)c3ccc(Cl)c(Cl)c3)[C@@]2(C)O1]

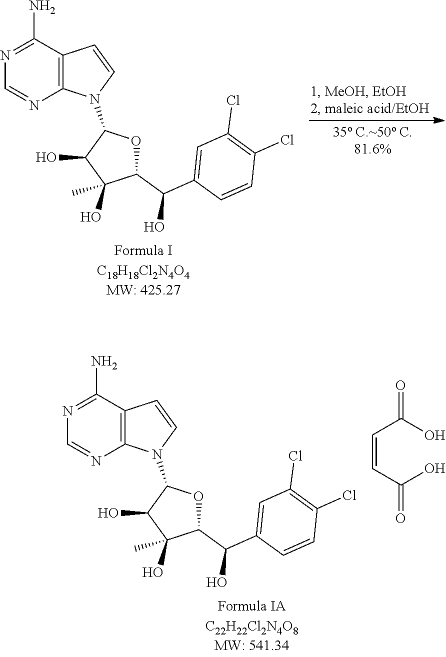

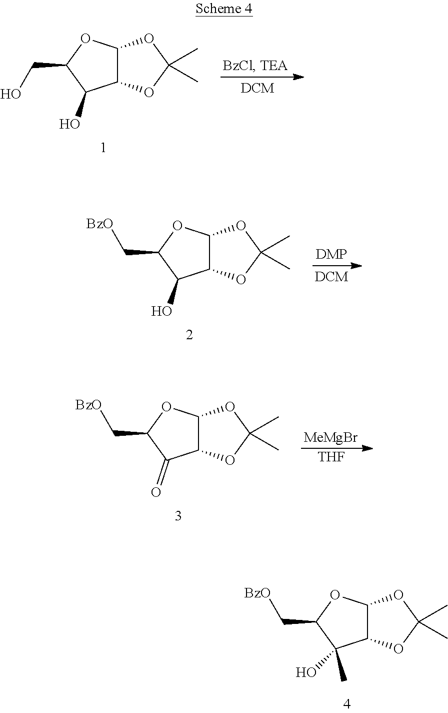

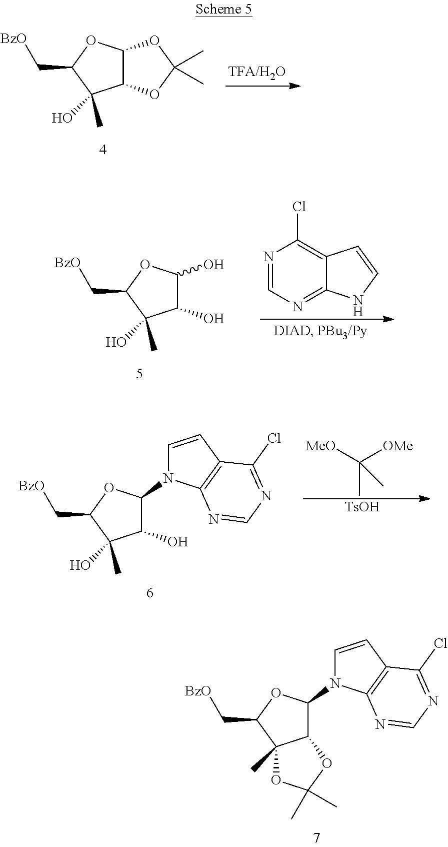

### Experimental Procedures

### Synthesis of 3

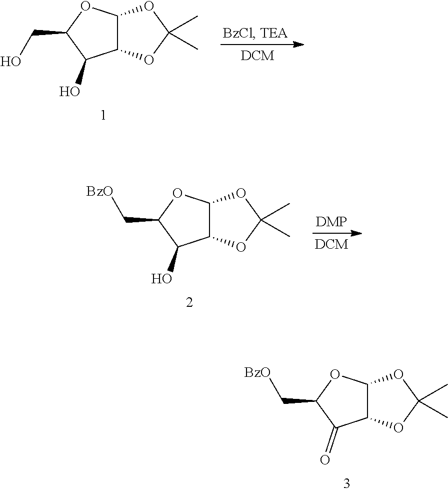

### Step 1. Synthesis of ((3aR,5R,6S,6aR)-6-hydroxy-2,2-dimethyltetrahydrofuro[2,3-d][1,3]dioxol-5-yl)methyl benzoate (2)

To a mixture of compound 1 (40.00 g, 210.31 mmol, 1 eq.) in DCM (400 mL) was added dropwise TEA (63.84 g, 630.94 mmol, 87.82 mL, 3 eq.) at 0° C. under N₂. BzCl (32.52 g, 231.34 mmol, 26.88 mL, 1.1 eq.) was added dropwise to the mixture at 0° C. under N₂. The mixture was stirred at 0° C. for 1 h under N₂. The mixture was combined another reaction mixture with 10 g of 1. The combined mixture was quenched by water (600 mL). The organic layer was separated. The aqueous was extracted with DCM (300 mL×3). The combined organic layers were washed with saturated NaHCO₃ solution (400 mL), dried over Na₂SO₄, filtered and concentrated. The residue was purified by column chromatography (SiO₂, Petroleum ether/Ethyl acetate=50/1 to 2/1) to give 2 (67.00 g, 227.66 mmol, 86.60% yield) as a yellow solid. ¹H NMR (400 MHz, CHLOROFORM-d) δ=8.12-7.95 (m, 2H), 7.66-7.53 (m, 1H), 7.51-7.41 (m, 2H), 5.97 (d, J=3.7 Hz, 1H), 4.87-4.75 (m, 1H), 4.60 (d, J=3.5 Hz, 1H), 4.47-4.35 (m, 2H), 4.19 (dd, J=2.2, 4.0 Hz, 1H), 3.27 (d, J=4.0 Hz, 1H), 1.52 (s, 3H), 1.33 (s, 3H).

### Step 2. Synthesis of ((3aR,5R,6aS)-2,2-dimethyl-6-oxotetrahydrofuro[2,3-d][1,3]dioxol-5-yl)methyl benzoate (3)

Two batches in parallel: To a mixture of compound 2 (10.00 g, 33.98 mmol, 1 eq.) in DCM (100 mL) was added DMP (43.24 g, 101.94 mmol, 31.56 mL, 3 eq.) at 0° C. The mixture was stirred at 15° C. for 4 h. The mixture was filtered and the filtrate was concentrated. The residue was diluted with EtOAc (500 mL) and the mixture was filtered. The filtrated was diluted with saturated NaHCO₃ (300 mL). The mixture was extracted with EtOAc (200 mL*3). The combined organic layers were washed with brine (300 mL), dried over Na₂SO₄, filtered and concentrated. The residue was purified by column chromatography (SiO₂, Petroleum ether/Ethyl acetate=20/1 to 3/1) to give 3 (17.00 g, 58.16 mmol, 85.59% yield) as a white solid. ¹H NMR (400 MHz, CHLOROFORM-d) δ=8.00-7.91 (m, 2H), 7.65-7.53 (m, 1H), 7.50-7.40 (m, 2H), 6.15 (d, J=4.4 Hz, 1H), 4.78-4.67 (m, 2H), 4.54-4.41 (m, 2H), 1.53 (s, 3H), 1.44 (s, 3H)

### Synthesis of Int-6

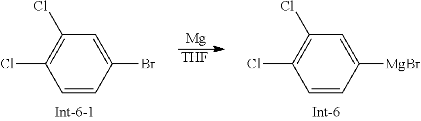

To a solution of Mg (979.09 mg, 40.28 mmol, 1.3 eq.) was added compound Int-6-1 (7 g, 30.99 mmol, 1 eq.) in THF (26 mL) at 40° C. under N₂. The mixture was stirred at 40° C. for 0.5 h. Mg was consumed. Compound Int-6 (7.75 g, crude) in THF (26 mL) was used into the next step without further purification as a yellow liquid.

### Preparation of ((3aR,5R,6R,6aR)-6-hydroxy-2,2,6-trimethyltetrahydrofuro[2,3-d][1,3]dioxol-5-yl)methyl benzoate (4)

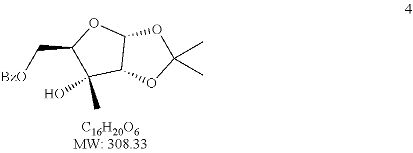

To a mixture of 3 (17.00 g, 58.16 mmol, 1 eq.) in THF (200 mL) was added dropwise MeMgBr (3 M, 58.16 mL, 3 eq.) at −78° C. under N₂. The mixture was stirred at −78° C. for 1 h under N₂. The combined mixture was quenched by saturated NH₄Cl (200 mL), extracted with EtOAc (50 mL*3). The combined organic layers were washed with brine (100 mL), dried over Na₂SO₄, filtered and concentrated. The crude product was purified by column chromatography (SiO₂, Petroleum ether/Ethyl acetate=15/1 to 5/1) to compound 4 as a white solid. ¹H NMR (400 MHz, CHLOROFORM-d) δ=8.13-8.01 (m, 2H), 7.64-7.51 (m, 1H), 7.48-7.38 (m, 2H), 5.83 (d, J=4.0 Hz, 1H), 4.57 (dd, J=3.1, 11.9 Hz, 1H), 4.38 (dd, J=8.2, 11.9 Hz, 1H), 4.21-4.06 (m, 2H), 2.71 (s, 1H), 1.60 (s, 3H), 1.37 (s, 3H), 1.26 (s, 3H).

### Preparation of ((3aR,4R,6R,6aR)-6-(4-chloro-7H-pyrrolo[2,3-d]pyrimidin-7-yl)-2,2,3a-trimethyltetrahydrofuro[3,4-d][1,3]dioxol-4-yl)methyl benzoate (7)

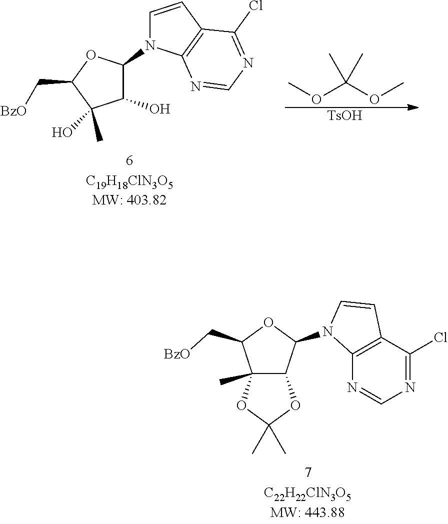

To a solution of compound 6 (1 g, 2.48 mmol, 1 eq.) in 2,2-dimethoxypropane (12.75 g, 122.42 mmol, 15 mL, 49.44 eq.) was added TsOH.H₂O (141.31 mg, 742.91 umol, 0.3 eq.). The mixture was stirred at 25° C. for 12 hr. LC-MS showed compound 6 was remained. Several new peaks were shown on LC-MS and desired compound was detected. The reaction was stirred at 60° C. for 2 hr. TLC indicated compound 6 was consumed completely and new spots formed. The reaction was clean according to TLC. The reaction was quenched by NaHCO₃ (20 mL), and extracted with EtOAc (10 mL*3). The organic was concentrated in vacuo. The residue was purified by column chromatography (SiO₂, Petroleum ether/Ethyl acetate=5/1 to 4:1). Compound 7 (730 mg, crude) was obtained as a yellow oil. TLC (Petroleum ether:Ethyl acetate=1:1) R_{f}=0.79.

### Preparation of ((3aR,4R,6R,6aR)-6-(4-chloro-7H-pyrrolo[2,3-d]pyrimidin-7-yl)-2,2,3a-trimethyltetrahydrofuro[3,4-d][1,3]dioxol-4-yl)methanol (8)

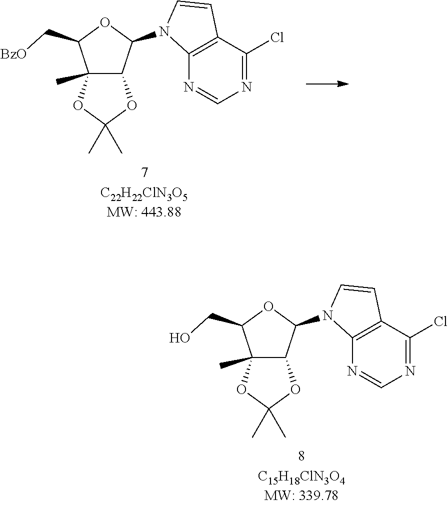

A mixture of compound 7 (600 mg, 1.35 mmol, 1 eq.) and NH₃ in MeOH (7 M, 10 mL, 51.79 eq.) was stirred at 25° C. for 12 h. LCMS showed the desired MS was observed. The mixture was concentrated. The residue was purified by column chromatography (SiO₂, Petroleum ether/Ethyl acetate=1/0 to 3:1). Compound 8 (450 mg, 1.32 mmol, 97.98% yield) was obtained as white solid. ¹H NMR (400 MHz, CHLOROFORM-d) δ=8.60 (s, 1H), 7.29 (d, J=3.7 Hz, 1H), 6.60 (d, J=3.7 Hz, 1H), 6.17 (d, J=3.2 Hz, 1H), 4.74 (d, J=3.1 Hz, 1H), 4.20 (dd, 5.6 Hz, 1H), 3.89-3.71 (m, 2H), 1.61 (s, 3H), 1.57 (s, 3H), 1.38 (s, 3H); LCMS: (M+H⁺): 340.1.

### Preparation of (3aS,4S,6R,6aR)-6-(4-chloro-7H-pyrrolo[2,3-d]pyrimidin-7-yl)-2,2,3a-trimethyltetrahydrofuro[3,4-d][1,3]dioxole-4-carboxylic acid (9)

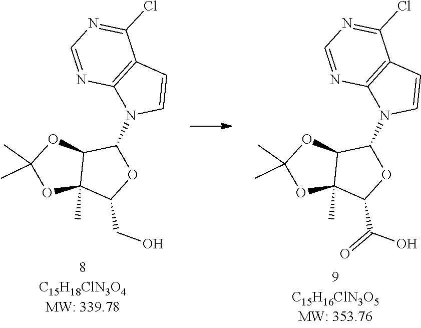 [SMILES: CC1(C)O[C@H]2[C@H](n3ccc4c(Cl)ncnc43)O[C@H](CO)[C@@]2(C)O1 ; CC1(C)O[C@H]2[C@H](n3ccc4c(Cl)ncnc43)O[C@H](C(=O)O)[C@@]2(C)O1]

To a mixture of compound 8 (500 mg, 1.47 mmol, 1 eq.), diacetoxyiodobenzene (DAIB) (1.04 g, 3.24 mmol, 2.2 eq.) in MeCN (2 mL) and H₂O (2 mL) was added TEMPO (46.28 mg, 294.31 umol, 0.2 eq.) at 0° C. The mixture was stirred at 25° C. for 1 h. TLC showed the compound 8 was consumed. The mixture was concentrated. The residue was dissolved in toluene (10 mL). The mixture was concentrated. The crude product was used for next step without further purification. Compound 9 (520 mg, crude) was obtained as brown oil. TLC (SiO₂, ethyl acetate/ethanol=1/1): R_{f}=0.5.

### Preparation of (3aS,4S,6R,6aR)-6-(4-chloro-7H-pyrrolo[2,3-d]pyrimidin-7-yl)-N-methoxy-N,2,2,3a-tetramethyltetrahydrofuro[3,4-d][1,3]dioxole-4-carboxamide (10)

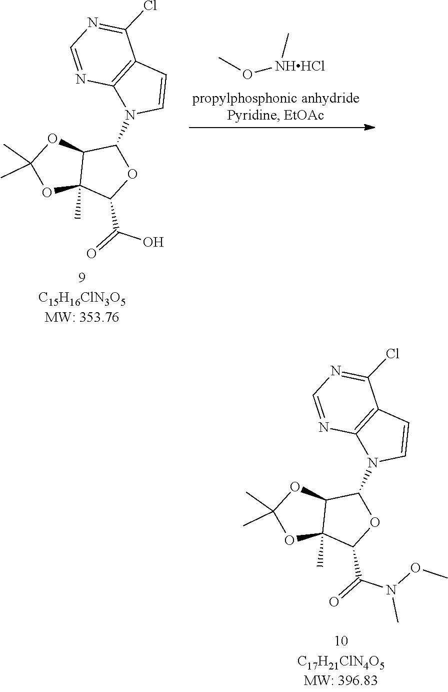

To a mixture of compound 9 (520 mg, 1.47 mmol, 1 eq.), N-methoxymethanamine (215.07 mg, 2.20 mmol, 1.5 eq., HCl), pyridine (348.82 mg, 4.41 mmol, 355.93 uL, 3 eq.) in EtOAc (5 mL) was added T3P (1.87 g, 2.94 mmol, 1.75 mL, 50% purity, 2 eq.) at 25° C. The mixture was stirred at 25° C. for 12 h. TLC showed the compound 9 was consumed. The mixture was quenched by water (50 mL) and extracted with EtOAc (25 mL×3). The combined organic layers were dried over Na₂SO₄, filtered and concentrated. The residue was purified by prep-TLC (SiO₂, Petroleum ether/Ethyl acetate=1/1). Compound 10 (450 mg, 1.13 mmol, 77.15% yield) was obtained as colorless oil. ¹H NMR (400 MHz, CHLOROFORM-d) δ=8.67 (s, 1H), 8.21 (d, J=3.7 Hz, 1H), 6.69-6.63 (m, 2H), 5.26 (s, 1H), 4.60 (d, J=1.3 Hz, 1H), 3.79 (s, 3H), 3.28 (s, 3H), 1.70 (s, 3H), 1.46 (d, J=3.5 Hz, 6H); LCMS: (M+H⁺): 397.2; TLC (SiO₂, petroleum ether/ethyl acetate=1/1): R_{f}=0.6.

### Preparation of ((3aS,4S,6R,6aR)-6-(4-chloro-7H-pyrrolo[2,3-d]pyrimidin-7-yl)-2,2,3a-trimethyltetrahydrofuro[3,4-d][1,3]dioxol-4-yl)(3,4-dichlorophenyl)methanone (11)

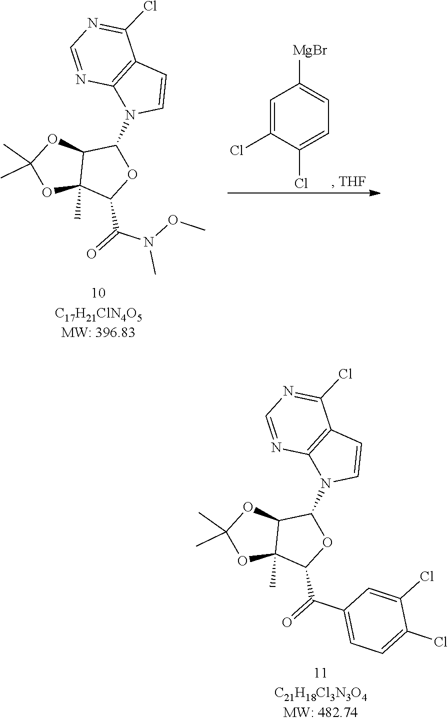

To a solution of compound 10 (1 g, 2.52 mmol, 1 eq.) in THF (15 mL) was added compound Int-6 (1 M, 10.08 mL, 4 eq.) at −10° C. under N₂. The mixture was stirred at 0° C. for 5 min. TLC indicated compound 10 was consumed completely and many new spots formed. The reaction was clean according to TLC (Petroleum ether:Ethyl acetate=3:1 R_{f}=0.48). The solution was added aq. sat. NH₄Cl (15 mL) and extracted with DCM (10 mL×2). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=1/0 to 15/1) and based on TLC (Petroleum ether:Ethyl acetate=3:1 R_{f}=0.48). Compound 11 (660 mg, 1.27 mmol, 50.42% yield, LCMS purity 92.94%) was obtained as a white solid. ¹H NMR (400 MHz, CHLOROFORM-d) δ=8.64-8.73 (m, 1H), 8.28 (d, J=2.19 Hz, 1H), 7.99 (dd, J=8.33, 2.19 Hz, 1H), 7.89 (d, J=3.95 Hz, 1H), 7.63 (d, J=8.33 Hz, 1H), 6.72 (d, J=3.95 Hz, 1H), 6.59 (d, J=1.32 Hz, 1H), 5.54 (s, 1H), 4.70 (d, J=1.32 Hz, 1H), 1.83 (s, 3H), 1.47 (s, 3H), 1.36 (s, 3H); LCMS: (M+H⁺): 483.9, LCMS purity 92.94%; TLC (Petroleum ether:Ethyl acetate=3:1) R_{f}=0.48.

### Preparation of (R)-((3aR,4R,6R,6aR)-6-(4-chloro-7H-pyrrolo[2,3-d]pyrimidin-7-yl)-2,2,3a-trimethyltetrahydrofuro[3,4-d][1,3]dioxol-4-yl)(3,4-dichlorophenyl)methanol (12)

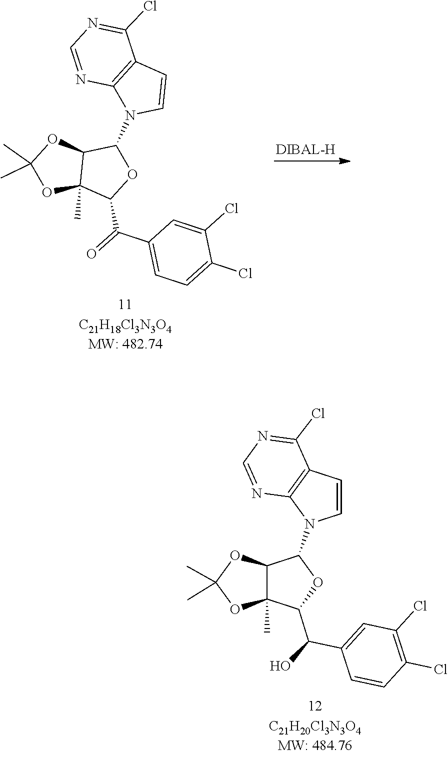 [SMILES: CC1(C)O[C@H]2[C@H](n3ccc4c(Cl)ncnc43)O[C@H](C(=O)c3ccc(Cl)c(Cl)c3)[C@@]2(C)O1 ; CC1(C)O[C@H]2[C@H](n3ccc4c(Cl)ncnc43)O[C@H]([C@H](O)c3ccc(Cl)c(Cl)c3)[C@@]2(C)O1]

To a solution of compound 11 (660 mg, 1.37 mmol, 1 eq.) in toluene (10 mL) was added DIBAL-H (1 M, 2.73 mL, 2 eq.) at −70° C. under N₂. The mixture was stirred at −70° C. for 5 min. TLC indicated compound 11 was consumed completely and one new spot formed. The reaction was clean according to TLC (Petroleum ether:Ethyl acetate=3:1 R_{f}=0.30). The reaction solution was added aq. sat. seignette salt (30 mL) and MTBE (20 mL) stirred at 25° C. for 0.5 h and extracted with MTBE (10 mL×4), washed with brine (10 mL×2), dried Na₂SO₄, filtered and concentrated under reduced pressure to give a residue. The residue was purified by column chromatography (SiO₂, Petroleum ether/Ethyl acetate=1/0 to 1/1) and based on TLC (Petroleum ether:Ethyl acetate=3:1 R_{f}=0.30). Compound 12 (310 mg, 513.06 umol, 37.53% yield, LCMS purity 80.23%) was obtained as a white solid. ¹H NMR (400 MHz, CHLOROFORM-d) δ=8.67 (s, 1H), 7.52 (d, J=1.75 Hz, 1H), 7.40 (d, J=8.33 Hz, 1H), 7.31 (d, J=3.51 Hz, 1H), 7.22 (dd, J=8.33, 1.75 Hz, 1H), 6.69 (d, J=3.95 Hz, 1H), 6.17 (d, J=2.63 Hz, 1H), 4.83 (d, J=8.33 Hz, 1H), 4.76 (d, J=2.63 Hz, 1H), 4.05-4.18 (m, 1H), 2.94 (br s, 1H), 1.84 (s, 3H), 1.67 (s, 3H), 1.43 (s, 3H); LCMS: (M+H⁺): 484.3. LCMS purity 80.23%; TLC (Petroleum ether:Ethyl acetate=3:1) R_{f}=0.30.

### Preparation of (R)-((3aR,4R,6R,6aR)-6-(4-amino-7H-pyrrolo[2,3-d]pyrimidin-7-yl)-2,2,3a-trimethyltetrahydrofuro[3,4-d][1,3]dioxol-4-yl)(3,4-dichlorophenyl)methanol (13)

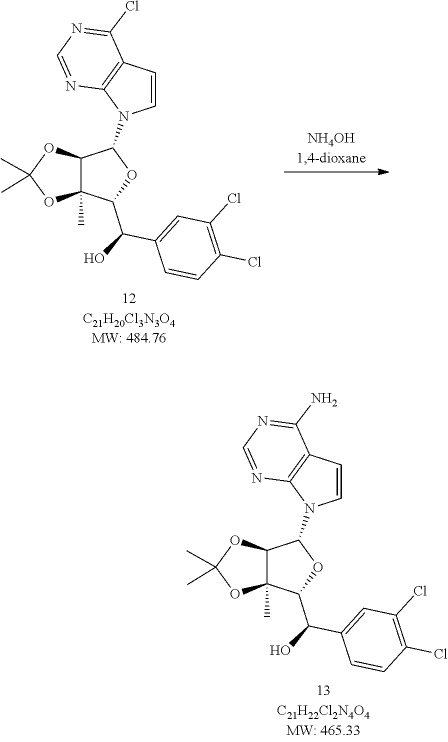 [SMILES: CC1(C)O[C@H]2[C@H](n3ccc4c(Cl)ncnc43)O[C@H]([C@H](O)c3ccc(Cl)c(Cl)c3)[C@@]2(C)O1 ; CC1(C)O[C@H]2[C@H](n3ccc4c(N)ncnc43)O[C@H]([C@H](O)c3ccc(Cl)c(Cl)c3)[C@@]2(C)O1]

To a solution of compound 12 (90 mg, 185.66 umol, 1 eq.) in dioxane (5 mL) was added NH₃H₂O (26.03 mg, 185.66 umol, 28.60 uL, 25% purity, 1 eq.) at 25° C. The mixture was sealed and stirred at 100° C. for 12 h (30 psi). LC-MS showed compound 12 was consumed completely and one main peak with desired product was detected. The reaction mixture was concentrated under reduced pressure to remove solvent. Compound 13 (80 mg, crude) was used into the next step without further purification as a yellow solid.

### Preparation of (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol (Formula I)

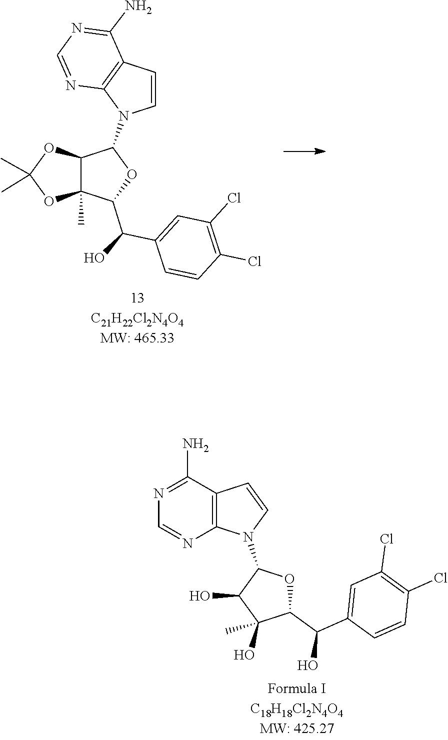 [SMILES: CC1(C)O[C@H]2[C@H](n3ccc4c(N)ncnc43)O[C@H]([C@H](O)c3ccc(Cl)c(Cl)c3)[C@@]2(C)O1 ; C[C@@]1(O)[C@@H]([C@H](O)c2ccc(Cl)c(Cl)c2)O[C@@H](n2ccc3c(N)ncnc32)[C@@H]1O]

To a solution of 13 (80 mg, 171.92 umol, 1 eq.) was added HCl/MeOH (4 M, 4.26 mL, 99.07 eq.) at 0° C. The mixture was stirred at 25° C. for 10 min. LC-MS showed no 13 was remained. Several new peaks were shown on LC-MS and desired compound was detected. The reaction mixture was concentrated under reduced pressure to remove solvent. The residue was added NH₃.H₂O to adjusted pH around 8. The residue was purified by prep-HPLC (basic condition column: Waters Xbridge 150*25 5u; mobile phase: [water (0.04% NH₃H₂O+10 mM NH₄HCO₃)-ACN]; B %: 15%-45%, 10 min). Formula I (29.83 mg, 69.48 umol, 40.41% yield, LCMS purity 99.05%) was obtained as a white solid. ¹H NMR (400 MHz, DMSO-d₆) δ=8.04 (s, 1H), 7.61 (d, J=1.75 Hz, 1H), 7.51 (d, J=8.77 Hz, 1H), 7.42 (d, J=3.51 Hz, 1H), 7.38 (dd, J=8.33, 1.75 Hz, 1H), 7.07 (br s, 2H), 6.55-6.64 (m, 2H), 5.85 (d, J=8.33 Hz, 1H), 5.27 (d, J=7.45 Hz, 1H), 4.78-4.86 (m, 2H), 4.43 (t, J=7.89 Hz, 1H), 4.01 (d, J=6.14 Hz, 1H), 1.18 (s, 3H); ¹H NMR (400 MHz, DMSO-d₆+D₂O) δ=8.03 (s, 1H), 7.58 (d, J=1.54 Hz, 1H), 7.50 (d, J=8.16 Hz, 1H), 7.34-7.41 (m, 2H), 6.58 (d, J=3.53 Hz, 1H), 5.84 (d, J=8.16 Hz, 1H), 4.80 (d, J=6.39 Hz, 1H), 4.41 (d, J=8.16 Hz, 1H), 4.00 (d, J=6.39 Hz, 1H), 1.18 (s, 3H); LCMS: (M+H⁺): 425.1. LCMS purity 99.05%; HPLC purity: 100.00%.

### Formula IA. (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol, maleate salt (IA)

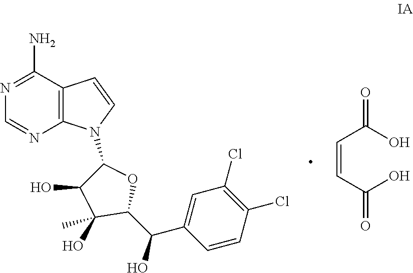 [SMILES: C[C@@]1(O)[C@@H]([C@H](O)c2ccc(Cl)c(Cl)c2)O[C@@H](n2ccc3c(N)ncnc32)[C@@H]1O ; O=C(O)/C=C\C(=O)O]

### Method 1:

(2R,3S,4R,5R)-5-(4-aminopyrrolo[2,3-d]pyrimidin-7-yl)-2-[(R)-(3,4-dichlorophenyl)-hydroxy-methyl]-3-methyl-tetrahydrofuran-3,4-diol (Formula I; 0.95 g, 2.24 mmol) was taken up in 120 mL of ACN:water (50:50) and heated until the solid dissolves. A solution of Maleic acid (260.1 mg, 2.24 mmol) in ACN:water (10 mL) was added and the resulting solution was cooled slowly. After 3 h very little solid had formed, and so the solution was concentrated to about 80 mL, cooled slowly, and allowed to stand overnight. A small amount of solids were filtered off (approx. 14 mg). The filtrate was concentrated to approximately 50 mL (ratio of ACN:water (50:50) has changed with greater concentration of water), seeded with crystals already collected, allowed to cool slowly. Allow to stand 3 h and filter solid (approx. 3.2 g after drying for 1 h (MP=201.2-201.5° C.). Dried in vacuum at room temperature overnight.

¹H NMR (500 MHz, DMSO-d₆) δ 8.19 (s, 1H), 7.81 (s, 1H), 7.61 (dd, J=2.8, 17.5 Hz, 2H), 7.50 (d, J=8.3 Hz, 1H), 7.36 (dd, J=2.0, 8.4 Hz, 1H), 6.76 (d, J=3.5 Hz, 1H), 6.35-6.19 (m, 1H), 6.14 (s, 2H), 5.92 (d, J=8.2 Hz, 1H), 5.40-5.23 (m, 1H), 4.88 (s, 1H), 4.79 (d, J=7.2 Hz, 1H), 4.37 (d, J=8.2 Hz, 1H), 3.97 (d, J=7.2 Hz, 1H), 1.23 (s, 3H).

Crystals are long narrow needles.

LCMS: RT=1.98 (424.8/428.8).

MP 201.6-202.7° C.

### Method 2:

To a clean container was added Formula I (100.0 g, 1 eq), followed by a mixed solution of acetonitrile (450 mL) and DI water (315 mL). The mixture was warmed to about 50° C. to a solution. It was filtered through a filter to give filtrate as clear solution A. This solution A was transferred into a clean 5 L RBF equipped with a mechanical stirrer, thermocouple and nitrogen inlet. The container used to make Formula I solution was washed with a mixed solution of acetonitrile (50 mL) and DI water (35 mL). This wash solution was filtered through the same filter and the filtrate was transferred into the 5 L of RBF. The batch in the 5 L RBF was heated to about 58° C. A prefiltered solution of maleic acid (30 g, 1.1 eq) in DI water (100 mL) was added to the 5 L RBF at the speed to maintain the internal temperature at 40-60° C. Then polish-filtered DI water (2000 mL) was added to the 5 L RBF at the speed to maintain the internal temperature at no less than 40° C. The batch in the 5 L RBF was allowed to cool to 15-25° C. and stirred overnight. The batch in the 5 L RBF was cooled to 0-10° C. and stirred for about 2 h. The batch in the 5 L RBF was filtered and the filter cake was washed with polish-filtered DI water (1000 mL). The filtered cake was dried on the filter for about 3.5 h. The product was transferred to tray and dried in oven under vacuum at 40° C. to constant weight (110 g). Yield for this production was 86.5%.

### Method 3:

Formula I free base is dissolved in methanol (12 volumes) at 20-45° C. The solution is polish-filtered through a filter loaded with celite (˜1 weight). Additional methanol (4 volumes) is used to wash. The filtrate and wash are transferred to a rotary evaporator through an in-line filter and concentrated on the rotary evaporator until the distillation stops. Filtered ethanol (3.5 volumes) is charged to the rotary evaporator and concentrated until distillation ceases. The solid (Formula I) is mixed in the rotary evaporator with filtered ethanol (10 volumes), the mixture is then transferred to a reactor and heated to 35-50° C. A polish-filtered solution of maleic acid (1.1 eq) in ethanol (3.5 volumes) is then added at 35-50° C. The batch is stirred at 35-50° C. for AO minutes, cooled to 15-30° C., then stirred at this temperature for A hours. The solid is filtered and the filter cake is washed with filtered ethanol (3.5 volumes). The product is dried by pulling air through the filter cake, then the product is transferred to drying trays and further dried under ambient air conditions. The product is further dried under vacuum at ≤45° C. until it reaches a constant weight. The product is ground with a spatula and passed through a 60-mesh sieve. The product is further dried in an oven under vacuum at ≤45° C. until it reaches constant weight. The resulting solid is Formula IA.

XRPD is shown in FIG. 1. DSC is shown in FIG. 3. TGA is shown in FIG. 4.

### Method 4:

Formula IA was prepared by placing Formula I free base into acetonitrile at an initial concentration of approximately 20 mg/mL. The sample was warmed to approximately 55° C. and one equivalent of maleic acid was added. The sample immediately gelled. Additional acetonitrile was added and finally a small quantity of water (final concentration of approximately 9 mg/mL in an 8:1 ACN/H2O (by volume) solution). The sample immediately clarified with the water addition. The sample was left for a slow cool procedure. No solids were generated from solution. The samples volume was dramatically reduced and then the sample was subjected to probe sonication. White solids precipitated from solution. The solids were collected by filtration.

XRPD is shown in FIG. 2. Table 8, below, shows the crystal data.

TABLE 8
FIG. 2 Crystal Data

| Bravais Type | Primitive Monoclinic |
|---|---|
| a [Å] | 12.298 |
| b [Å] | 6.993 |
| c [Å] | 28.585 |
| α [deg] | 90 |
| β [deg] | 98.30 |
| γ [deg] | 90 |
| Volume [Å³/cell] | 2,432.5 |
| Chiral Contents? | Chiral |
| Extinction Symbol | P 1 2₁ 1 |
| Space Group(s) | P2₁ (4) |

DSC and TGA are shown in FIG. 5.

Gravimetric solubility estimates were carried out on this material in water and found to be approximately 1.1 g/L.

### Method 5:

To 30.5 mg of maleic acid (0.263 mmol, 1.05 eq.) was added 106.6 mg (0.25 mmol, 1.0 eq.) of Formula I. 4.0 mL of EtOH was added and the resulting mixture was stirred continuously overnight. The mixture was filtered to give a solid, which was washed with 2.5 mL MTBE, and then dried (40° C. under vacuum overnight) to give Formula IA.

XRPD is shown in FIG. 14.

DSC is shown in FIG. 15.

TGA is shown in FIG. 16.

### Formula IB. (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol, hydrochloride salt (IB)

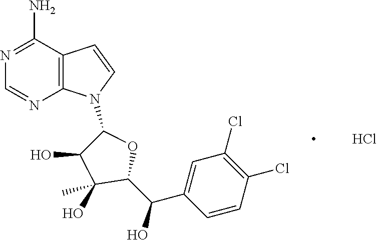 [SMILES: C[C@@]1(O)[C@@H]([C@H](O)c2ccc(Cl)c(Cl)c2)O[C@@H](n2ccc3c(N)ncnc32)[C@@H]1O ; Cl]

### Method 1:

Crystalline Formula IB was generated from an experiment which combined Formula I and aqueous HCl (1 eq.) in acetonitrile (ACN) at elevated temperature. The reagents were in a 1:1 molar ratio and, once a clear solution was obtained, the solution was allowed to cool to ambient temperature. The solids were collected and characterized after drying under ambient conditions.

XRPD is shown in FIG. 8. Table 9, below, shows the crystal data.

TABLE 9
FIG. 8 Crystal Data

| Bravais Type | Primitive Orthorhombic |
|---|---|
| a [Å] | 9.597 |
| b [Å] | 13.189 |
| c [Å] | 35.618 |
| α [deg] | 90 |
| β [deg] | 90 |
| γ [deg] | 90 |
| Volume [Å³/cell] | 4,508.3 |
| Chiral Contents? | Chiral |
| Extinction Symbol | P 2₁ 2₁ 2₁ |
| Space Group(s) | P2₁2₁2₁ (19) |

DSC and TGA are shown in FIG. 11.

Gravimetric solubility estimates were carried out on this material in water and found to be approximately 0.8 g/L.

### Method 2:

(2R,3S,4R,5R)-5-(4-aminopyrrolo[2,3-d]pyrimidin-7-yl)-2-[(R)-(3,4-dichlorophenyl)-hydroxy-methyl]-3-methyl-tetrahydrofuran-3,4-diol (Formula I; 201.0 mg, 0.47 mmol) is taken up in ACN (5 mL) and the mixture is heated until the solids dissolves. A solution of hydrochloric acid (0.03 mL, 0.47 mmol) in 1 mL ACN is added and the solution is cooled slowly. Filtered off solid, dried in vacuo.

MP darkens and shrinks at 210.6-212.8° C., melts 216.9-217.9° C.

Cl titration found: 23.13%. theory 23.03%

¹H NMR (500 MHz, DMSO-d₆) δ 8.35 (s, 1H), 7.85 (d, J=3.7 Hz, 1H), 7.58 (d, J=2.0 Hz, 1H), 7.49 (d, J=8.3 Hz, 1H), 7.35 (dd, J=2.0, 8.4 Hz, 1H), 7.01 (d, J=3.7 Hz, 1H), 6.00 (d, J=8.2 Hz, 1H), 5.92 (s, 1H), 4.78 (d, J=7.9 Hz, 1H), 4.33 (d, J=8.2 Hz, 1H), 3.93 (d, J=7.9 Hz, 1H), 2.06 (s, 1H), 1.29 (s, 3H).

XRPD is shown in FIG. 6.

DSC is shown in FIG. 9.

TGA is shown in FIG. 10.

### Method 3:

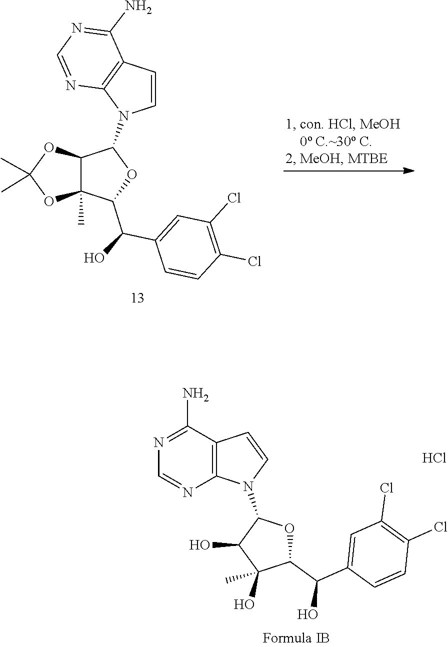 [SMILES: CC1(C)O[C@H]2[C@H](n3ccc4c(N)ncnc43)O[C@H]([C@H](O)c3ccc(Cl)c(Cl)c3)[C@@]2(C)O1 ; C[C@@]1(O)[C@@H]([C@H](O)c2ccc(Cl)c(Cl)c2)O[C@@H](n2ccc3c(N)ncnc32)[C@@H]1O ; Cl]

Concentrated hydrochloric acid (36.5-38.0%, 15 eq) is added to a pre-cooled (0-10° C.) solution of 13 in methanol (10 volumes) while maintaining the temperature at 10° C. The batch is warmed to 20˜30° C. and stirred at this temperature range for hours. The reaction continues until the in-process control criterion (≤1.0% 13 vs Formula IB by HPLC) is met. The batch is filtered and the filter cake (Formula IB) is washed with ethanol. The filter cake is dried on the funnel by pulling air through the cake for hour.

XRPD is shown in FIG. 7.

### Method 4—Formula IB, Form I:

A slurry of about 40 mg of Formula IB in 0.6 mL of ethyl formate was stirred at 55° C. for over a weekend, and then filtered and washed with 0.6 of MTBE to give crystalline Formula IB, Form I, which was dried in an oven at 47-48° C. overnight.

XRPD is shown in FIG. 20.

DSC is shown in FIG. 21.

TGA is shown in FIG. 22.

Karl-Fisher titration indicated that the Formula IB, Form I contains about 0.21% water.

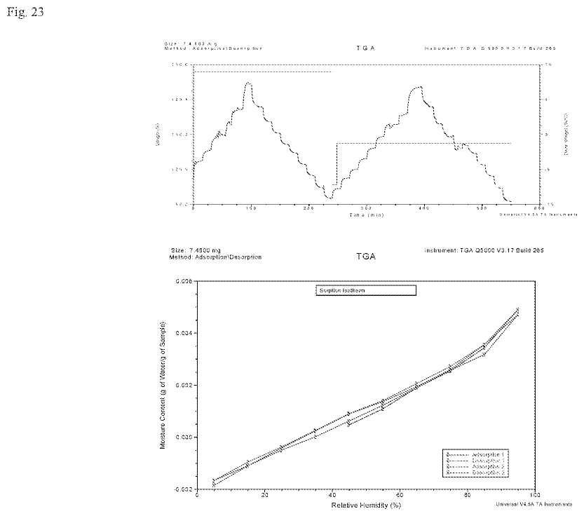

The adsorption/desorption isotherms of Formula IB, Form I, shown in FIG. 23, indicates that it can adsorb ˜0.5% water at about 95% humidity and can adsorb ˜0.1% of the water at room temperature and normal humidity range (40-50% RH).

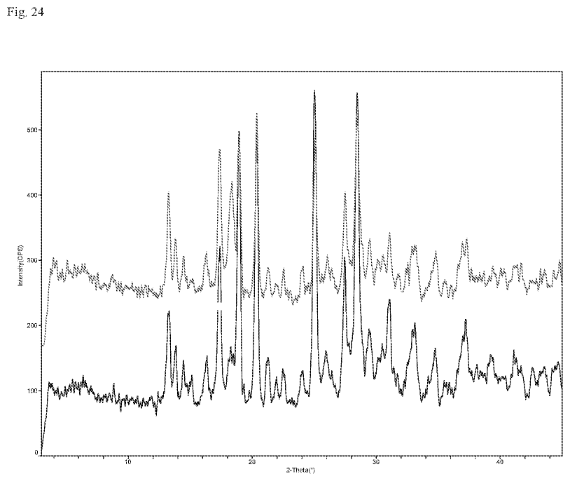

Comparison of XRPD before and after DVS showed no change in Form. See FIG. 24.

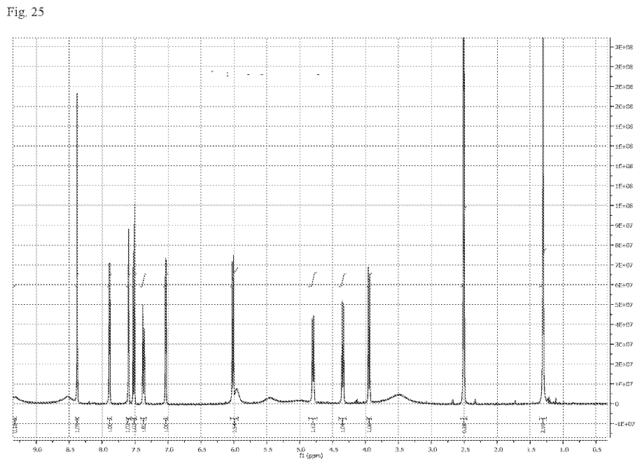

¹H NMR is shown in FIG. 25.

Method 5: Formula IB, Form II

A slurry of 60 mg of Formula IB in 1.2 mL of ethanol was stirred at 55° C. for 16 h, filtered, and the solids washed with 1.0 mL of MTBE. The solids were dried in an oven at 47-48° C. overnight to give Formula IB, Form II.

XRPD is shown in FIG. 26.

DSC is shown in FIG. 27

TGA is shown in FIG. 28.

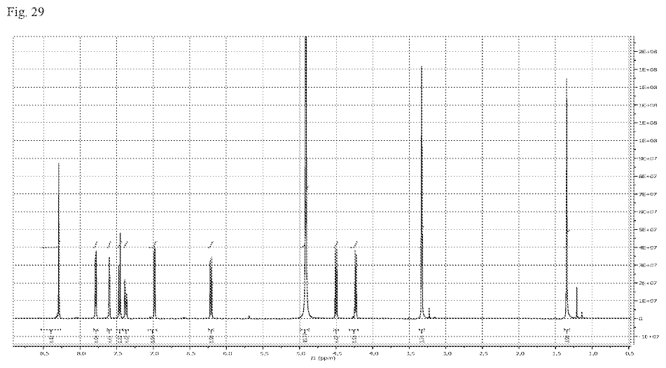

¹H NMR is shown in FIG. 29.

The Karl-Fisher titration indicated that the Formula IB, Form II contains about 0.54% water.

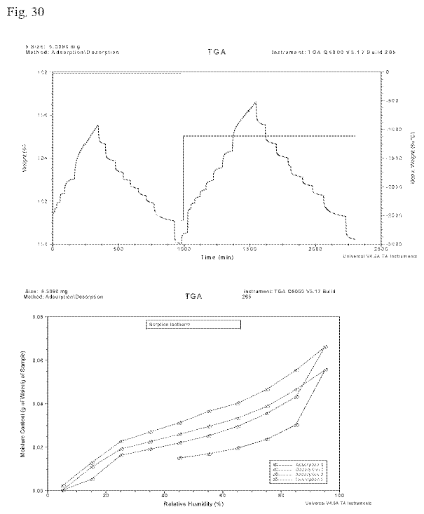

DVS is shown in FIG. 30. The adsorption/desorption isotherms of Formula IB, Form II indicated that it could adsorb ˜6% water at about 95% humidity and can adsorb ˜3% of the water at room temperature and normal humidity range (40-50% RH).

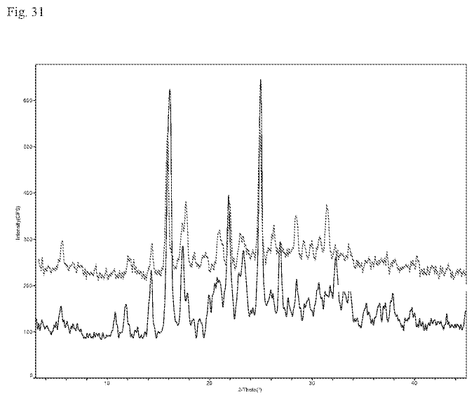

Comparison of XRPD before and after DVS showed no change in Form. See FIG. 31.

Method 6: Formula IB, Form III

A slurry of about 35 mg of Formula IB in 0.4 mL of acetone was stirred at 55° C. for the over a weekend, and then filtered and washed with 0.5 of MTBE to give crystalline Formula IB, Form III which was dried in an oven at 47-48° C. overnight.

XRPD is shown in FIG. 32.

DSC is shown in FIG. 33.

TGA is shown in FIG. 34. The sample exhibited approximately 0.01% of weight loss up to about 100° C.

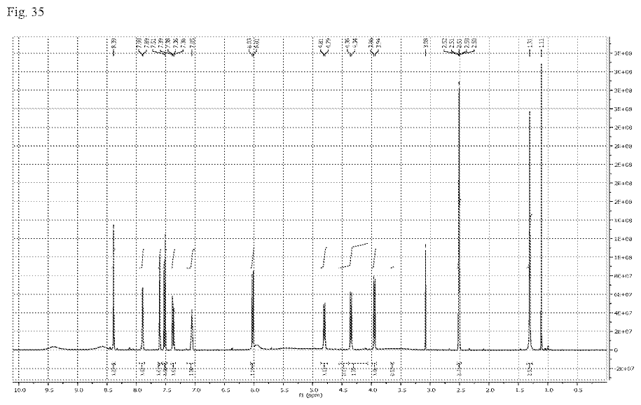

¹H NMR in FIG. 35.

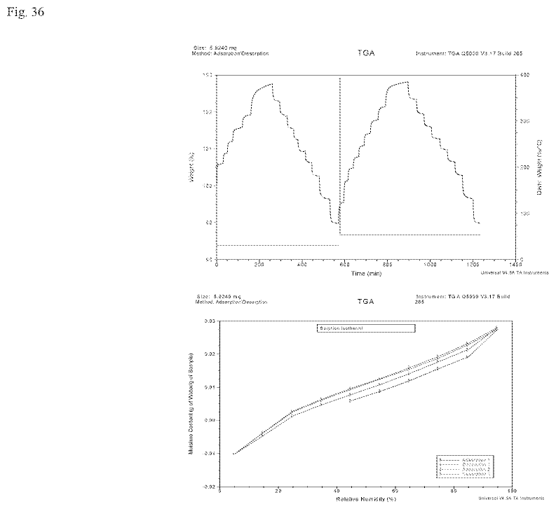

DVS is shown in FIG. 36. The adsorption/desorption isotherms of Formula IB, Form III indicates that it could adsorb ˜2.8% water at about 95% humidity and can adsorb ˜1% of the water at room temperature and normal humidity range (40-50% RH).

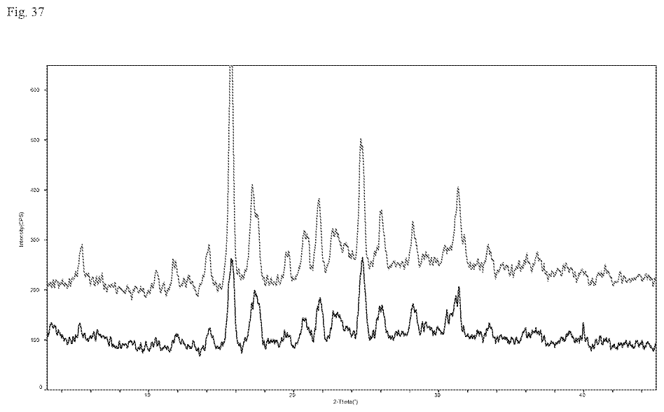

FIG. 37. The XRPD before and after DVS showed no change in form.

### Method 7: Formula IB, Form IV

A slurry of 40 mg of Formula IB in 0.6 mL of n-propanol was stirred at 55° C. for over weekend, filtered, washed with 0.5 mL of MTBE, and dried under oven at 47-48° C. overnight to give the Formula IB, Form IV.

XRPD is shown in FIG. 38.

DSC is shown in FIG. 39. The DSC indicates an onset temperature at 214.32° C. and a peak at 220.59° C.

TGA is shown in FIG. 40. The TGA shows approximately 0.02% of weight loss up to about 130° C.

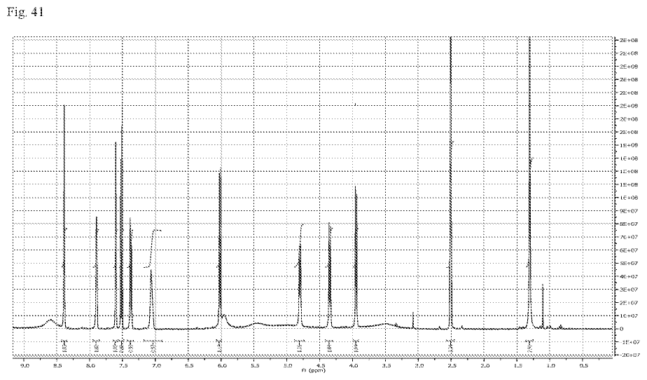

¹H NMR in FIG. 41.

### Method 8:

To 106.3 mg of Formula I (0.25 mmol, 1.0 eq.) was added 4.0 mL of 2-butanone and the resulting mixture was stirred for 5 minutes. 263 μL of 1.0 M HCl in IPA (0.263 mmol, 1.06 eq.) was added. The mixture was stirred to give a thin slurry, which was continuously stirred overnight. The mixture was filtered to give a solid which was dried (40° C. under vacuum overnight) to give Formula IB (97 mg, 85.8% yield).

XRPD is shown in FIG. 17.

DSC is shown in FIG. 18.

TGA is shown in FIG. 19.

### Method 9:

210 mg of Formula I free base (0.494 mmol, 1.0 eq.) and 5.0 mL of methanol were stirred to give a clear solution. Hydrochloric acid (0.51 mL, 1.03 eq., in IPA from 37% aqueous solution) was added and the mixture was stirred for about 1.0 min to give a slurry. The slurry was continuously stirred for 2.0 h, then at 50° C. for 1.0 h, then at room temperature for 1.0 h. The mixture was filtered and washed with MTBE (4.0 mL) and the solids dried at 45-48° C., under vacuum for 24 h.

XRPD is shown in FIG. 42.

DSC is shown in FIG. 43.

TGA is shown in FIG. 44.

### Formula IC. (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol, oxalate salt (IC)

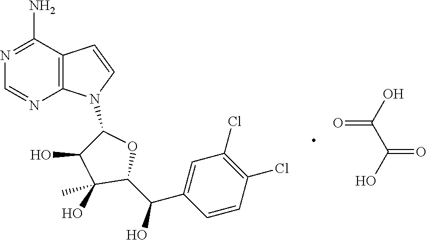 [SMILES: C[C@@]1(O)[C@@H]([C@H](O)c2ccc(Cl)c(Cl)c2)O[C@@H](n2ccc3c(N)ncnc32)[C@@H]1O ; O=C(O)C(=O)O]

Crystalline Formula IC was generated from an experiment which combined Formula I and oxalic acid (1 eq.) in ethanol at elevated temperature. The solution was allowed to cool and then the ethanol was allowed to evaporate. The solids were collected and characterized after drying under ambient conditions.

XRPD is shown in FIG. 12. Table 10, below, shows the crystal data.

TABLE 10
FIG. 12 Crystal Data

| Bravais Type | Primitive Orthorhombic |
|---|---|
| a [Å] | 7.373 |
| b [Å] | 11.580 |
| c [Å] | 50.309 |
| α [deg] | 90 |
| β [deg] | 90 |
| γ [deg] | 90 |
| Volume [Å/cell] | 4,295.3 |
| Chiral Contents? | Chiral |
| Extinction Symbol | P 2₁ 2₁ 2₁ |
| Space Group(s) | P2₁2₁2₁ (19) |

Gravimetric solubility estimates were carried out on this material in water and no solubility was detected (<0.3 g/L).

### Formula ID. (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol, phosphate salt (ID)

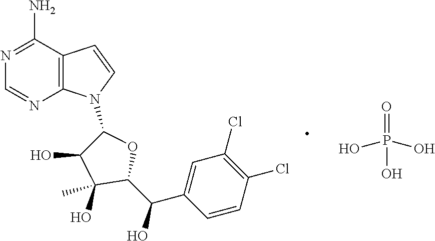 [SMILES: C[C@@]1(O)[C@@H]([C@H](O)c2ccc(Cl)c(Cl)c2)O[C@@H](n2ccc3c(N)ncnc32)[C@@H]1O ; O=P(O)(O)O]

### Method 1:

Crystalline Formula ID was generated from an experiment which combined Formula I and phosphoric acid (1 eq.) in ethanol at elevated temperature. The sample was allowed to cool and solids precipitated from solution. The solids were collected and characterized after drying under ambient conditions.

XRPD is shown in FIG. 13. Table 11, below, shows the crystal data.

TABLE 11
FIG. 13 Crystal Data

| Bravais Type | Primitive Orthorhombic |
|---|---|
| a [Å] | 7.730 |
| b [Å] | 12.120 |
| c [Å] | 49.420 |
| α [deg] | 90 |
| β [deg] | 90 |
| γ [deg] | 90 |
| Volume [Å³/cell] | 4,630.0 |
| Chiral Contents? | Chiral |
| Extinction Symbol | P 2₁ 2₁ 2₁ |
| Space Group(s) | P2₁2₁2₁ (19) |

Gravimetric solubility estimates were carried out on this material in water and no solubility was detected (<0.3 g/L).

### Method 2:

To 106.7 mg of Formula I (0.25 mmol, 1.0 eq.) was added 4.0 mL of MeOH and the resulting mixture was stirred to afford a clear solution. 265 μL of 1.0 M H₃PO₄ in IPA (0.265 mmol, 1.06 eq.) was added. The mixture was stirred continuously overnight, and then filtered to give a solid, which was dried (40° C. under vacuum overnight) to give Formula IC.

XRPD is shown in FIG. 45.

DSC is shown in FIG. 46.

TGA is shown in FIG. 47.

### Formula IE. (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol, bisulfate (IE)

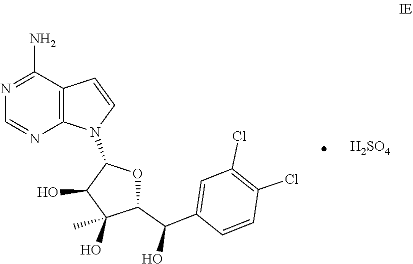 [SMILES: C[C@@]1(O)[C@@H]([C@H](O)c2ccc(Cl)c(Cl)c2)O[C@@H](n2ccc3c(N)ncnc32)[C@@H]1O ; "H2SO4"]

To (2R,3S,4R,5R)-5-(4-aminopyrrolo[2,3-d]pyrimidin-7-yl)-2-[(R)-(3,4-dichlorophenyl)-hydroxy-methyl]-3-methyl-tetrahydrofuran-3,4-diol (100 mg, 0.24 mmol) in IPA (5 mL) was sonicated at 50° C. to get a clear solution and then was added the sulfuric acid (2.14 mL, 0.24 mmol) and again sonicated at 50° C. for 5 mins. The mixture was allowed to cool slowly and solid obtained was centrifuged, washed with minimal amount of water and dried under high vacuum to give 95 mg of needle like crystals; m.p. 216-219° C. ¹H NMR (500 MHz, DMSO-d₆) δ 8.21 (s, 1H), 7.65 (d, J=3.7 Hz, 1H), 7.60 (d, J=1.9 Hz, 1H), 7.51 (d, J=8.3 Hz, 1H), 7.37 (dd, J=1.9, 8.3 Hz, 1H), 6.79 (d, J=3.6 Hz, 1H), 6.24 (br s, 1H), 5.94 (d, J=8.2 Hz, 1H), 5.33 (br s, 1H), 4.90 (br s, 1H), 4.80 (d, J=7.2 Hz, 1H), 4.44-4.33 (m, 1H), 3.98 (d, J=7.2 Hz, 1H), 1.25 (s, 3H).

### Formula I. (2R,3S,4R,5R)-5-(4-amino-7H-pyrrolo[2,3-d]pyrimidin-7-yl)-2-((R)-(3,4-dichlorophenyl)(hydroxy)methyl)-3-methyltetrahydrofuran-3,4-diol

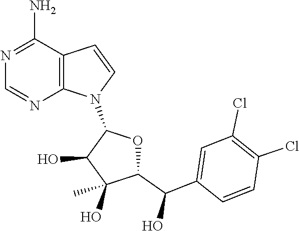 [SMILES: C[C@@]1(O)[C@@H]([C@H](O)c2ccc(Cl)c(Cl)c2)O[C@@H](n2ccc3c(N)ncnc32)[C@@H]1O]

### Method 1: Formula I, Form I

Formula I free base (56 mg, 0.132 mmol) and 1.0 mL of iso-propanol were stirred for 10 min to give a clear solution, which was stirred at 55° C. for 2.0 h, and then at room temperature for 4.0 h. The resulting solids were filtered, washed with MTBE (1.0 mL), and then dried at 46-48° C., under vacuum overnight to give 48.7 mg (86.96% yield) of Formula I crystalline Form I.

XRPD is shown in FIG. 48.

DSC is shown in FIG. 49.

TGA is shown in FIG. 50.

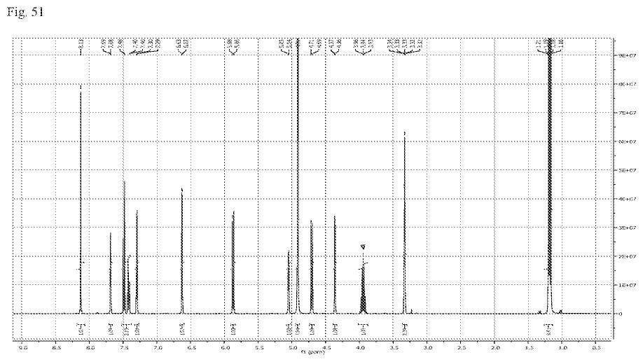

¹H NMR, shown in FIG. 51, indicates that Formula I, Form I is a mono-isopropanol solvate.

DVS is shown in FIG. 52.

XRPD before and after DVS, shown in FIG. 53, indicates no change in form.

Karl-Fisher titration indicated that the Formula I-Form I contains about 1.3% water.

The adsorption/desorption isotherms of Formula I Form I from IPA (FIG. 52) indicate that the crystalline form can adsorb ˜0.5% water at about 95% humidity and can adsorb ˜0.8% of the water at room temperature and normal humidity range (40-50% RH).

### Method 2: Formula I, Form 1

Formula I free base (175 mg, 0.412 mmol) and 2.5 mL of iso-propanol were stirred for 6 min to give a clear cream, which gave a slurry after continuous stirring for 10 minutes. The slurry was stirred at 50° C. for 2.5 h, and then at room temperature for 1.0 h. The mixture was filtered, washed with MTBE (2.0 mL), and then dried at 46-48° C., under vacuum overnight to yield 157 mg (89.71% yield) of Formula I, Form 1.

### Method 3: Formula I, Form II:

A slurry of Formula I free base (about 50 mg) in THF, was stirred for 4 h, then continuously stirred at 55° C. for 2 h, and then stirred at 25° C. for 4 h. The resulting mixture was filtered, washed with MTBE, and dried under oven at 45-46° C. for 24 h to give Formula I, Form II.

XRPD is shown in FIG. 58.

DSC is shown in FIG. 59.

### Method 4: Formula I, Form II:

A slurry of Formula I free base (about 50 mg) in Me-THF, was stirred for 4 h, then continuously stirred at 55° C. for 2 h, and then stirred at 25° C. for 4 h. The resulting mixture was filtered, washed with MTBE, and dried under oven at 45-46° C. for 24 h to give Formula I, Form II.

XRPD is shown in FIG. 60.

DSC is shown in FIG. 61.

### Method 5: Formula I, Form II:

A slurry of Formula I free base (about 50 mg) in acetone, was stirred for 4 h, then continuously stirred at 55° C. for 2 h, and then stirred at 25° C. for 4 h. The resulting mixture was filtered, washed with MTBE, and dried under oven at 45-46° C. for 24 h to give Formula I, Form II.

XRPD is shown in FIG. 54.

DSC is shown in FIG. 55.

### Method 6: Formula I, Form II

Formula I free base (150 mg, 0.353 mmol) and 2.0 mL of ethanol were stirred for about 1.0 min to give a clear solution, which after 3 min gave a slurry. The slurry was continuously stirred for 5 min, then at 55° C. for 2.5 h, then room temperature for 1.0 h. The mixture was filtered and washed with MTBE (2.0 mL) and the solids were dried at 46-48° C., under vacuum overnight to give 121 mg, (80.7% yield) of Formula I, Form II.

XRPD is shown in FIG. 62.

DSC is shown in FIG. 63.

### Method 7: Formula I, Form III

A slurry of Formula I free base in methanol/water (1/5) was stirred for 10 min, then at 55° C. for 2 h and then at room temperature for 1 h. The mixture was filtered, and the solids were washed with MTBE, and then dried under vacuum at 47-48° C. overnight to give Formula I, Form III.

XRPD is shown in FIG. 56.

DSC is shown in FIG. 57.

### Instrument Methods

### X-Ray Powder Diffraction (XRPD)

XRPD patterns can be collected with a PANalytical X'Pert PRO MPD diffractometer using an incident beam of Cu radiation produced using an Optix long, fine-focus source. An elliptically graded multilayer mirror is used to focus Cu Kα X-rays through the specimen and onto the detector. Prior to the analysis, a silicon specimen (NIST SRM 640e) is analyzed to verify the observed position of the Si 111 peak is consistent with the NIST-certified position. A specimen of the sample is sandwiched between 3-μm-thick films and analyzed in transmission geometry. A beam-stop, short antiscatter extension, and antiscatter knife edge is used to minimize the background generated by air. Soller slits for the incident and diffracted beams are used to minimize broadening from axial divergence. Diffraction patterns are collected using a scanning position-sensitive detector (X'Celerator) located 240 mm from the specimen and Data Collector software v. 2.2b.

XRPD patterns also can be collected with a Rigaku MiniFlex X-ray Powder Diffractometer (XRPD) instrument. X-ray radiation is from Copper (Cu) at 1.54056 Å with K_{b} filter. X-ray power: 30 KV, 15 mA.

### Thermogravimetric Analysis (TGA) and Differential Scanning Calorimetry (DSC)

Thermal analysis can be performed using a Mettler Toledo TGA/DSC3+ analyzer. Temperature calibration is performed using phenyl salicylate, indium, tin, and zinc. The sample is placed in an aluminum pan. The sample is sealed, the lid pierced, then inserted into the TG furnace. The furnace is heated under nitrogen.

DSC can also be obtained using a TA Instrument Differential Scanning calorimetry, Model Q20 with autosampler, using a scan rate of 10° C./min, and nitrogen gas flow at 50 mL/min.

TGA can be collected using a TGA Q500 by TA Instruments using a scan rate of 20° C. per minute.

### Dynamic Vapor Sorption (DVS)

The dynamic vapor sorption experiments can be done with a VTI SGA-Cx100 Symmetric Vapor Sorption Analyzer. The moisture uptake profile is completed in three cycles of 10% RH increments with adsorption from 5% to 95% RH, followed by desorption of 10% increments from 95% to 5%. The equilibration criteria are 0.0050 wt % in 5 minutes with a maximum equilibration time of 180 minutes. All adsorption and desorption are performed at room temperature (21-22° C.). No pre-drying step is applied for the samples.

### Biochemical Assay Protocol

Compounds are solubilized and 3-fold diluted in 100% DMSO. These diluted compounds are further diluted in the assay buffer (50 mM Tris-HCl, pH 8.5, 50 mM NaCl, 5 mM MgCl₂, 0.01% Brij35, 1 mM DTT, 1% DMSO) for 10-dose IC₅₀ mode at a concentration 10-fold greater than the desired assay concentration. Standard reactions are performed in a total volume of 50 μl in assay buffer, with histone H2A (5 μM final) as substrate. To this was added the PRMT5/MEP50 complex diluted to provide a final assay concentration of 5 nM and the compounds are allowed to preincubate for 15 to 20 minutes at room temperature. The reaction is initiated by adding S-[3H-methyl]-adenosyl-L-methionine (PerkinElmer) to final concentration of 1 μM. Following a 60 minutes incubation at 30° C., the reaction is stopped by adding 100 μL of 20% TCA. Each reaction is spotted onto filter plate (MultiScreen FB Filter Plate, Millipore), and washed 5 times with PBS buffer, Scintillation fluid is added to the filter plate and read in a scintillation counter. IC₅₀ values are determined by fitting the data to the standard 4 parameters with Hill Slope using GraphPad Prism software.

### Cellular Assay Protocol

Cell Treatment and Western Blotting for Detecting Symmetric Di-Methyl Arginine (sDMA) and Histone H3R8 Dimethyl Symmetric (H3R8me2s) Marks

Initial compounds screening in A549 cells: Compounds are dissolved in DMSO to make 10 mM stock and further diluted to 0.1, and 1 mM. A549 cells are maintained in PRMI 1640 (Corning Cellgro, Catalog #: 10-040-CV) medium supplemented with 10% v/v FBS (GE Healthcare, Catalog #: SH30910.03). One day before experiment, 1.25×10⁵ cells are seeded in 6 well plate in 3 mL medium and incubated overnight. The next day, medium is changed and 3 uL of compound solution is added (1:1,000 dilution, 0.1 and 1 uM final concentration; DMSO concentration: 0.1%), and incubated for 3 days. Cells incubated with DMSO are used as a vehicle control. Cells are washed once with PBS, trypsinized in 150 uL 0.25% Trypsin (Corning, Catalog #: 25-053-CI), neutralized with 1 mL complete medium, transferred to microCentrifuge tubes and collected. Cell pellet is then resuspended in 15 uL PBS, lysed in 4% SDS, and homogenized by passing through homogenizer column (Omega Biotek, Catalog #: HCR003). Total protein concentrations are determined by BCA assay (ThermoFisher Scientific, Catalog #: 23225). Lysates are mixed with 5× Laemmli buffer and boiled for 5 min. Forty ug of total protein are separated on SDS-PAGE gels (Bio-Rad, catalog #: 4568083, 4568043), transferred to PVDF membrane, blocked with 5% dry milk (Bio-Rad, Catalog #: 1706404) in TBS with 0.1% v/v Tween 20 (TBST) for 1 hour at room temperature (RT), and incubated with primary antibodies (sDMA: Cell signaling, Catalog #: 13222, 1:3,000; H3R8me2s: Epigentek, Catalog #: A-3706-100, 1:2,000; β-Actin: Abcam, Catalog #: ab8227, 1:10,000) in 5% dry milk in TBST at 4° C. for overnight. The next day, membranes are washed with TBST, 5×5 min, and incubated with HRP conjugated seconded antibody (GE Healthcare; Catalog #: NA934-1ML; 1:5,000) for 2 hours at RT, followed by 5×5 min washes with TBST, and incubation with ECL substrates (Bio-Rad, Catalog #: 1705061, 1705062). Chemiluminescent signal is captured with Fluochem HD2 imager (Proteinsimple) and analyzed by ImageJ.

To determine enzyme inhibition IC₅₀ values using Western Blot analysis, Granta cells are seeded at density of 5×10⁵ cells/mL in 3 mL medium (PRMI+10% v/v FBS). Nine-point 3-fold serial dilutions of compound are added to cells (3 ul, 1:1,000 dilution, DMSO concentration is 0.1%; final top concentration is 10 or 1 uM, depending on compounds potency) and incubated for 3 days. Cells incubated with DMSO are used as a vehicle control. Cells are harvested and subjected to western blot analysis as described above. SmD3me2s and H3R8me2s bands are quantified by ImageJ. Signals are normalized to β-Actin and DMSO control. IC₅₀ values are calculated using Graphpad Prism.

### Cell Proliferation Assay to Determine IC₅₀ on Granta-519 Cells

Granta-519 cells are maintained in PRMI 1640 (Corning Cellgro, Catalog #: 10-040-CV) medium supplemented with 10% v/v FBS (GE Healthcare, Catalog #: SH30910.03). Formula I is dissolved in DMSO to make 10 mM stocks and stored at −20° C. Nine-point, 3-fold serial dilutions are made with DMSO with top concentration at 1 mM (working stocks).

On day of experiment, compound working stocks are further diluted at 1:50 with fresh medium in 96 well plate, and 10 μL of diluted drugs are added to a new 96 well plate for proliferation assay. Cells growing at exponential phase are spun down at 1500 rpm for 4 min and resuspend in fresh medium to reach a density of 0.5×10⁶ cells/ml. 200 ul of cells are added to 96 well plate containing diluted drugs and incubated for 3 days. DMSO is used a vehicle control.

One day 3, 10 μL of Cell Counting Kit-8 (CCK-8, Jojindo, CK04-13) solution is added to a new 96 well plate. Cells incubated with drugs for 3 days are resuspended by pipetting up and down, and 100 μL of cells are transferred to 96 well plate containing CCK-8 reagent to measure viable cells. Plates are incubated in CO2 incubator for 2 hours and OD450 values are measured with a microplate reader (iMark microplate reader, Bio-Rad).

For re-plating, compound working stocks are diluted at 1:50 with fresh medium and 10 μL of diluted drugs are added to a new 96 well plate. Cells from Day 3 plate (50 ul) are added to 96 well plate containing fresh drug and additional 150 μL of fresh medium are added to reach 200 μL volume. Plate is returned to CO₂ incubator and incubated for 3 more days. Viable cells measurement and re-plating are repeated on day 6, and the final viable cells measurement is taken on day 10.

Percentage of viable cells, relative to DMSO vehicle control, is calculated and plotted in Graphpad Prism ([Inhibitor] vs. normalized response−Variable slope) to determine proliferation IC₅₀ values on day 10.

TABLE A
Biochemical and cellular potency (in Granta-519 cell line)

| | PRMT5 | PRMT5 | sDMA IC₅₀ | sDMA | Prolif. | Prolif. |
|---|---|---|---|---|---|---|
| Ex# | IC₅₀ μM | IC₅₀_N | μM | ICs₅₀_N | IC₅₀ μM | IC₅₀_N |
| Formula | 0.0015 | 3 | 0.031 | 3 | 0.075 | 3 |
| I | | | | | | |

### FaSSIF Solubility of Formula IE

Compounds are first dispersed in freshly prepared FaSSIF (http://biorelevant.com/site_media/upload/documents/How_to_make_FaSSIF_FeSSIF_and_FaSSGF.pdf) buffer in 1 mg/mL respectively, and the standard samples are prepared by preparing 1 mg/mL of test compounds in DMSO. The compounds are then sufficient mixed by vortex mixer for 30 sec, and agitated at 25° C. using 300 rpm form 4 hour in thermo mixer. After incubation, the prepared samples are centrifuged at 10000 rpm for 10 min to remove the undissolved solid, the resulting supernatants are applied to HPLC. The actual concentrations of the compounds are evaluated by measuring the peak area, and the solubility (S) of compounds is calculated according to following equation:

S=Cₛₘₚ=C_{std}*(Aₛₘₚ/A_{std})*(V_{std}/Vₛₘₚ)

Where C is the sample concentration in μg/mL, A is the peak area, and V is the injection volume.
Warfarin (10-25 μg/mL), Atovaquone (<2 μg/mL) and Nimesulide (100-200 μg/mL) are positive controls in this experiment.

Formula IE was measured to have a FaSSIF solubility of 206 μg/mL.

### In Vivo Pharmacokinetic Properties of Formula I.

In a rat (SD, male, non-fasted) non-crossover PK study, the compound of Formula I was dosed at 1 mg/kg (DMA: 20% HPBCD=5:95, solution) via i.v. administration (N=3) and 1 mg/kg (0.5% Na CMC+0.5% Tween80, solution) via oral gauge (p.o.) (N=3). It showed average T_{1/2} of 4.1 hr, Vss of 3.1 L/kg, blood clearance of 8.8 mL/min/kg in the i.v. group; it showed average dose normalized AUC of 3246 ng*h*kg/mL/mg and >100% of oral bioavailability in the p.o. group.

### In Vivo Pharmacodynamic Effect and Tumor Growth Inhibition of Formula I in Granta-519 Mouse Xenograft Model.

Granta-519 cells was maintained in DMEM medium supplemented with 10% fetal bovine serum and 2 mM L-Glutamine at 37° C. in an atmosphere of 5% CO₂ in air. Cells in exponential growth phase were harvested and 1×10⁷ cells in 0.1 mL of PBS with Matrigel (1:1) were injected subcutaneously at the right lower flank region of each mouse for tumor development. The treatments were started when the mean tumor size reaches approximately 300-400 mm³. Mice were assigned into groups using StudyDirector™ software (Studylog Systems, Inc. CA, USA) and one optimal randomization design (generated by either Matched distribution or Stratified method) that shows minimal group to group variation in tumor volume was selected for group allocation. Formula I or vehicle (0.5% Na CMC+0.5% Tween80, suspension) were administered orally (QD for Formula I, QD for vehicle) at a dose of 30 mg/kg and 50 mg/kg for 19 and 16 days, respectively. Body weights and tumor size were measured every 3 to 4 days after randomization. Animals were euthanized 12 hours after last dosing, and blood and tumor samples were collected for analysis.

To measure sDMA levels in tumor samples, tumors from each mouse were weighted and homogenized in RIPA buffer supplemented with protease inhibitor (cOmplete™, EDTA-free Protease Inhibitor Cocktail, Roche). Lysate were centrifuged at 14,000 rpm for 30 min at 4° C. to remove debris. Total protein concentrations of lysate were determined by BCA assay (ThermoFisher Scientific, Catalog #: 23225). Equal amount of total proteins from each tumor were separated on SDS-PAGE gel, and sDMA levels were determined by WB as described previously.

Following this protocol, Formula I showed an average of 46% (N=5) tumor growth inhibition at 30 mg/kg with body weight loss of 1%; an average of 79% tumor growth inhibition of at 50 mg/kg with body weight loss of 8%. It also showed >90% inhibition of sDMA at 30 mg/kg and no detectable sDMA at 50 mg/kg.

The disclosure is also directed to the following aspects:
Aspect 1. A pharmaceutically acceptable salt of a compound of Formula I

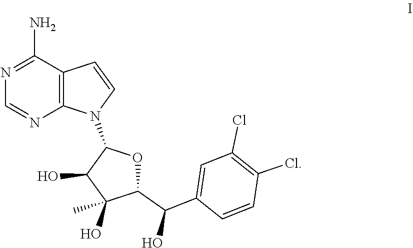 [SMILES: C[C@@]1(O)[C@@H]([C@H](O)c2ccc(Cl)c(Cl)c2)O[C@@H](n2ccc3c(N)ncnc32)[C@@H]1O]

Aspect 2. The pharmaceutically acceptable salt of aspect 1, wherein the salt is the maleate salt having Formula IA

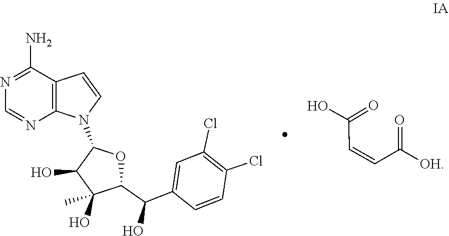 [SMILES: C[C@@]1(O)[C@@H]([C@H](O)c2ccc(Cl)c(Cl)c2)O[C@@H](n2ccc3c(N)ncnc32)[C@@H]1O ; O=C(O)/C=C\C(=O)O]

Aspect 3. A crystalline form of the pharmaceutically acceptable salt of aspect 2.
Aspect 4. The crystalline form of aspect 3, characterized by an X-ray powder diffraction pattern substantially as shown in FIG. 1.
Aspect 5. The crystalline form of aspect 3, characterized by an X-ray powder diffraction pattern substantially as shown in FIG. 2.
Aspect 6. The crystalline form of aspect 3, characterized by an X-ray powder diffraction pattern comprising a peak at 16.3 degrees±0.2 degrees 2-theta, on the 2-theta scale with lambda=1.54 angstroms (Cu Kα).
Aspect 7. The crystalline form of aspect 3, characterized by an X-ray powder diffraction pattern comprising peaks at 6.7, 11.0, and 16.3 degrees±0.2 degrees 2-theta, on the 2-theta scale with lambda=1.54 angstroms (Cu Kα).
Aspect 8. The crystalline form of aspect 3, characterized by an X-ray powder diffraction pattern comprising peaks at 6.7, 16.3, 20.4, and 30.7 degrees±0.2 degree 2-theta, on the 2-theta scale with lambda=1.54 angstroms (Cu Kα).
Aspect 9. The crystalline form of aspect 3, characterized by an X-ray powder diffraction pattern comprising peaks at 6.7, 11.0, 14.9, 16.3, 16.8, 20.4, 25.4 degrees±0.2 degree 2-theta, on the 2-theta scale with lambda=1.54 angstroms (Cu Kα).
Aspect 10. The crystalline form of aspect 3, characterized by an X-ray powder diffraction pattern comprising peaks at three or more of 6.7, 11.0, 14.9, 16.3, 16.8, 20.4, 25.4, 25.8, 27.9, 29.1, and 30.7 degrees±0.2 degrees 2-theta, on the 2-theta scale with lambda=1.54 angstroms (Cu Kα).
Aspect 11. The crystalline form of any one of aspects 3, 4 or 6 to 10, characterized by a differential scanning calorimetry (DSC) thermogram substantially as shown in FIG. 3 when heated at a rate of 10° C./min.
Aspect 12. The crystalline form of any one of aspects 3, 4 or 6 to 11, characterized by a differential scanning calorimetry (DSC) thermogram comprising an endothermic peak at about 207° C. when heated at a rate of 10° C./min.
Aspect 13. The crystalline form of aspect 3, characterized by an X-ray powder diffraction pattern comprising a peak at 14.6 degrees±0.2 degrees 2-theta, on the 2-theta scale with lambda=1.54 angstroms (Cu Kα).
Aspect 14. The crystalline form of aspect 3, characterized by an X-ray powder diffraction pattern comprising peaks at 13.0, 14.6, and 16.3 degrees±0.2 degrees 2-theta, on the 2-theta scale with lambda=1.54 angstroms (Cu Kα).
Aspect 15. The crystalline form of aspect 3, characterized by an X-ray powder diffraction pattern comprising peaks at 8.3, 13.0, 14.6, 16.3, 26.3, and 27.0 degrees±0.2 degree 2-theta, on the 2-theta scale with lambda=1.54 angstroms (Cu Kα).
Aspect 16. The crystalline form of aspect 3, characterized by an X-ray powder diffraction pattern comprising peaks at 8.3, 13.0, 14.6, 15.3, 16.3, 16.7, 27.0, and 27.2 degrees±0.2 degree 2-theta, on the 2-theta scale with lambda=1.54 angstroms (Cu Kα).
Aspect 17. The crystalline form of aspect 3, characterized by an X-ray powder diffraction pattern comprising peaks at three or more of 3.1, 8.3, 13.0, 14.6, 15.3, 16.3, 16.7, 18.4, 26.3, 26.5, 27.0, and 27.2 degrees±0.2 degrees 2-theta, on the 2-theta scale with lambda=1.54 angstroms (Cu Kα).
Aspect 18. The crystalline form of any one of aspects 3, 5, or 13 to 17 characterized by a differential scanning calorimetry (DSC) thermogram substantially as shown in FIG. 5 when heated at a rate of 10° K/min.
Aspect 19. The crystalline form of any one of aspects 3, 5, or 13 to 18, characterized by a differential scanning calorimetry (DSC) thermogram comprising an endothermic peak at about 185° C. when heated at a rate of 10° K/min.
Aspect 20. The crystalline form of any one of aspects 3, 4 or 6 to 12, characterized by a thermogravimetric analysis profile substantially as shown in FIG. 4 when heated at a rate of 20° C./min.
Aspect 21. The crystalline form of any one of aspects 3, 5, or 13 to 19, characterized by a thermogravimetric analysis profile substantially as shown in FIG. 5 when heated at a rate of 10° K/min.
Aspect 22. The pharmaceutically acceptable salt of aspect 1, wherein the salt is the hydrochloride salt having Formula IB

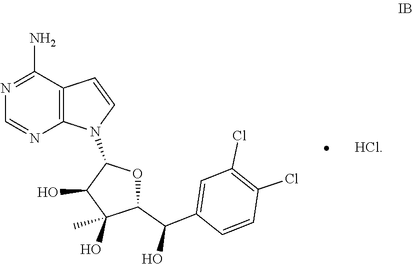 [SMILES: C[C@@]1(O)[C@@H]([C@H](O)c2ccc(Cl)c(Cl)c2)O[C@@H](n2ccc3c(N)ncnc32)[C@@H]1O ; "HCl."]

Aspect 23. A crystalline form of the pharmaceutically acceptable salt of aspect 22.
Aspect 24. The crystalline form of aspect 23, characterized by an X-ray powder diffraction pattern substantially as shown in FIG. 6.
Aspect 25. The crystalline form of one of aspect 23 or aspect 24, characterized by an X-ray powder diffraction pattern comprising a peak at 5.4 degrees±0.2 degrees 2-theta, on the 2-theta scale with lambda=1.54 angstroms (Cu Kα).
Aspect 26. The crystalline form of one of aspect 23 or aspect 24, characterized by an X-ray powder diffraction pattern comprising peaks at 5.4, 10.9, and 16.4 degrees±0.2 degrees 2-theta, on the 2-theta scale with lambda=1.54 angstroms (Cu Kα).
Aspect 27. The crystalline form of one of aspect 23 or aspect 24, characterized by an X-ray powder diffraction pattern comprising peaks at 5.4, 10.9, 21.2, and 24.2 degrees±0.2 degree 2-theta, on the 2-theta scale with lambda=1.54 angstroms (Cu Kα).
Aspect 28. The crystalline form of one of aspect 23 or aspect 24, characterized by an X-ray powder diffraction pattern comprising peaks at 5.4, 10.9, 16.4, 21.2, and 24.2 degrees±0.2 degree 2-theta, on the 2-theta scale with lambda=1.54 angstroms (Cu Kα).
Aspect 29. The crystalline form of one of aspect 23 or aspect 24, characterized by an X-ray powder diffraction pattern comprising peaks at three or more of 5.4, 10.9, 16.4, 21.2, 24.2, and 27.5 degrees±0.2 degrees 2-theta, on the 2-theta scale with lambda=1.54 angstroms (Cu Kα).
Aspect 30. The crystalline form of aspect 23, characterized by an X-ray powder diffraction pattern substantially as shown in FIG. 7.
Aspect 31. The crystalline form of one of aspect 23 or aspect 30, characterized by an X-ray powder diffraction pattern comprising a peak at 5.0 degrees±0.2 degrees 2-theta, on the 2-theta scale with lambda=1.54 angstroms (Cu Kα).
Aspect 32. The crystalline form of one of aspect 23 or aspect 30, characterized by an X-ray powder diffraction pattern comprising peaks at 5.0, 15.2, and 24.3 degrees±0.2 degrees 2-theta, on the 2-theta scale with lambda=1.54 angstroms (Cu Kα).
Aspect 33. The crystalline form of one of aspect 23 or aspect 30, characterized by an X-ray powder diffraction pattern comprising peaks at 5.0, 15.2, 24.3, and 30.8 degrees±0.2 degree 2-theta, on the 2-theta scale with lambda=1.54 angstroms (Cu Kα).
Aspect 34. The crystalline form of one of aspect 23 or aspect 30, characterized by an X-ray powder diffraction pattern comprising peaks at 5.0, 10.1, 13.7, 15.2, 17.1, 24.3, and 30.8 degrees±0.2 degree 2-theta, on the 2-theta scale with lambda=1.54 angstroms (Cu Kα).
Aspect 35. The crystalline form of aspect 23, characterized by an X-ray powder diffraction pattern substantially as shown in FIG. 8.
Aspect 36. The crystalline form of one of aspect 23 or aspect 35, characterized by an X-ray powder diffraction pattern comprising a peak at 11.4 degrees±0.2 degrees 2-theta, on the 2-theta scale with lambda=1.54 angstroms (Cu Kα).
Aspect 37. The crystalline form of one of aspect 23 or aspect 35, characterized by an X-ray powder diffraction pattern comprising peaks at 11.4, 11.6, 15.1, and 16.7 degrees±0.2 degrees 2-theta, on the 2-theta scale with lambda=1.54 angstroms (Cu Kα).
Aspect 38. The crystalline form of one of aspect 23 or aspect 35, characterized by an X-ray powder diffraction pattern comprising peaks at 4.9, 11.4, 11.6, 15.1, and 16.7 degrees±0.2 degree 2-theta, on the 2-theta scale with lambda=1.54 angstroms (Cu Kα).
Aspect 39. The crystalline form of one of aspect 23 or aspect 35, characterized by an X-ray powder diffraction pattern comprising peaks at 4.9, 11.4, 11.6, 15.1, 16.7, 21.0, and 22.4 degrees±0.2 degree 2-theta, on the 2-theta scale with lambda=1.54 angstroms (Cu Kα).
Aspect 40. The crystalline form of one of aspect 23 or aspect 35, characterized by an X-ray powder diffraction pattern comprising peaks at three or more of 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degrees 2-theta, on the 2-theta scale with lambda=1.54 angstroms (Cu Kα).
Aspect 41. The crystalline form of any one of aspects 23 to 29, characterized by a differential scanning calorimetry (DSC) thermogram substantially as shown in FIG. 9 when heated at a rate of 10° C./min.
Aspect 42. The crystalline form of any one of aspects 23 or 35 to 40, characterized by a differential scanning calorimetry (DSC) thermogram substantially as shown in FIG. 11 when heated at a rate of 10° C./min.
Aspect 43. The crystalline form of any one of aspects 23 to 29, or 41, characterized by a differential scanning calorimetry (DSC) thermogram comprising an endothermic peak at about 268° C. when heated at a rate of 10° C./min.
Aspect 44. The crystalline form of any one of aspects 23 to 29, 41, or 43, characterized by a differential scanning calorimetry (DSC) thermogram comprising an endothermic peak at about 191° C. when heated at a rate of 10° C./min.
Aspect 45. The crystalline form of any one of aspects 23, 35 to 40, or 42, characterized by a differential scanning calorimetry (DSC) thermogram comprising an endothermic peak at about 196° C. when heated at a rate of 10° C./min.
Aspect 46. The crystalline form of any one of aspects 23 to 29, 41, or 43, or 44, characterized by a thermogravimetric analysis profile substantially as shown in FIG. 10 when heated at a rate of 20° C./min.
Aspect 47. The crystalline form of any one of aspects 23, 35 to 40, or 42, characterized by a thermogravimetric analysis profile substantially as shown in FIG. 11 when heated at a rate of 10° C./min.
Aspect 48. The pharmaceutically acceptable salt of aspect 1, wherein the salt is the oxalate salt having Formula IC

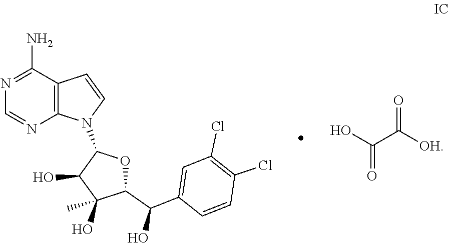 [SMILES: C[C@@]1(O)[C@@H]([C@H](O)c2ccc(Cl)c(Cl)c2)O[C@@H](n2ccc3c(N)ncnc32)[C@@H]1O ; O=C(O)C(=O)O]

Aspect 49. A crystalline form of the pharmaceutically acceptable salt of aspect 48.
Aspect 50. The crystalline form of aspect 49, characterized by an X-ray powder diffraction pattern substantially as shown in FIG. 12.
Aspect 51. The crystalline form of one of aspect 49 or aspect 50, characterized by an X-ray powder diffraction pattern comprising a peak at 10.5 degrees±0.2 degrees 2-theta, on the 2-theta scale with lambda=1.54 angstroms (Cu Kα).
Aspect 52. The crystalline form of one of aspect 49 or aspect 50, characterized by an X-ray powder diffraction pattern comprising peaks at 10.5, 14.7, and 16.2 degrees±0.2 degrees 2-theta, on the 2-theta scale with lambda=1.54 angstroms (Cu Kα).
Aspect 53. The crystalline form of one of aspect 49 or aspect 50, characterized by an X-ray powder diffraction pattern comprising peaks at 10.5, 14.7, 16.2, and 28.7 degrees±0.2 degree 2-theta, on the 2-theta scale with lambda=1.54 angstroms (Cu Kα).
Aspect 54. The crystalline form of one of aspect 49 or aspect 50, characterized by an X-ray powder diffraction pattern comprising peaks at 10.5, 14.7, 16.2, 17.6, 17.7, 19.6, 28.7, and 28.9 degrees±0.2 degree 2-theta, on the 2-theta scale with lambda=1.54 angstroms (Cu Kα).
Aspect 55. The crystalline form of one of aspect 49 or aspect 50, characterized by an X-ray powder diffraction pattern comprising peaks at three or more of 10.5, 11.6, 13.1, 14.2, 14.7, 14.9, 16.2, 17.6, 17.7, 19.6, 28.7, and 28.9 degrees±0.2 degrees 2-theta, on the 2-theta scale with lambda=1.54 angstroms (Cu Kα).
Aspect 56. The pharmaceutically acceptable salt of aspect 1, wherein the salt is the phosphate salt having Formula ID

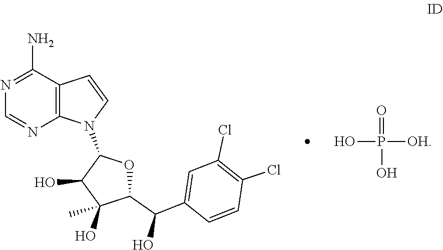 [SMILES: C[C@@]1(O)[C@@H]([C@H](O)c2ccc(Cl)c(Cl)c2)O[C@@H](n2ccc3c(N)ncnc32)[C@@H]1O ; O=P(O)(O)O]

Aspect 57. A crystalline form of the pharmaceutically acceptable salt of aspect 56.
Aspect 58. The crystalline form of aspect 57, characterized by an X-ray powder diffraction pattern substantially as shown in FIG. 13.
Aspect 59. The crystalline form of one of aspect 57 or aspect 58, characterized by an X-ray powder diffraction pattern comprising a peak at 3.6 degrees±0.2 degrees 2-theta, on the 2-theta scale with lambda=1.54 angstroms (Cu Kα).
Aspect 60. The crystalline form of one of aspect 57 or aspect 58, characterized by an X-ray powder diffraction pattern comprising peaks at 3.6, and 10.7 degrees±0.2 degrees 2-theta, on the 2-theta scale with lambda=1.54 angstroms (Cu Kα).
Aspect 61. The crystalline form of one of aspect 57 or aspect 58, characterized by an X-ray powder diffraction pattern comprising peaks at 3.6, 10.7, and 15.6 degrees±0.2 degree 2-theta, on the 2-theta scale with lambda=1.54 angstroms (Cu Kα).
Aspect 62. The crystalline form of one of aspect 57 or aspect 58, characterized by an X-ray powder diffraction pattern comprising peaks at three or more of 3.6, 10.7, 15.6, 17.9, and 18.7 degrees±0.2 degrees 2-theta, on the 2-theta scale with lambda=1.54 angstroms (Cu Kα).
Aspect 63. The pharmaceutically acceptable salt of aspect 1, wherein the salt is the bisulfate salt having Formula IE

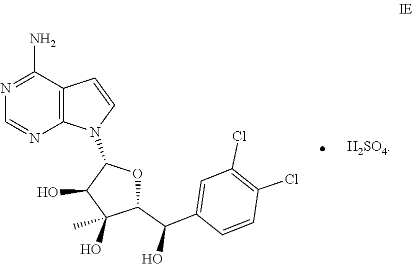 [SMILES: C[C@@]1(O)[C@@H]([C@H](O)c2ccc(Cl)c(Cl)c2)O[C@@H](n2ccc3c(N)ncnc32)[C@@H]1O ; "H2SO4."]

Aspect 64. A crystalline form of the pharmaceutically acceptable salt of aspect 63.
Aspect 65. A pharmaceutical composition comprising a pharmaceutically acceptable salt according to any one of aspects 1 to 64, and a pharmaceutically acceptable excipient.
Aspect 66. A method of inhibiting a protein arginine methyltransferase 5 (PRMT5) enzyme, comprising: contacting the PRMT5 enzyme with an effective amount of a compound of any one of aspects 1 to 64.
Aspect 67. A method of treating a disease or disorder associated with aberrant PRMT5 activity in a subject comprising administering to the subject, a compound of any one of aspects 1 to 64.
Aspect 68. The method of aspect 67, wherein the disease or disorder associated with aberrant PRMT5 activity is breast cancer, lung cancer, pancreatic cancer, prostate cancer, colon cancer, ovarian cancer, uterine cancer, cervical cancer, leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), myelodysplastic syndrome (MDS), epidermoid cancer, or hemoglobinopathies such as b-thalassemia and sickle cell disease (SCD).
Aspect 69. The method of aspect 67 or aspect 68, wherein the compound, or a pharmaceutically acceptable salt thereof, is administered in combination with one or more other agents.

1-162. (canceled)

163. A pharmaceutically acceptable salt of a compound of Formula I

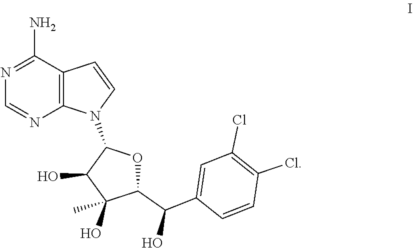 [SMILES: C[C@@]1(O)[C@@H]([C@H](O)c2ccc(Cl)c(Cl)c2)O[C@@H](n2ccc3c(N)ncnc32)[C@@H]1O]

164. The pharmaceutically acceptable salt of claim 163, wherein the salt is the maleate salt having Formula IA

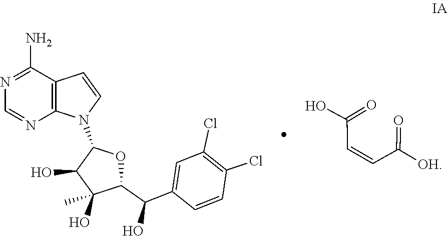 [SMILES: C[C@@]1(O)[C@@H]([C@H](O)c2ccc(Cl)c(Cl)c2)O[C@@H](n2ccc3c(N)ncnc32)[C@@H]1O ; O=C(O)/C=C\C(=O)O]

165. A crystalline form of the pharmaceutically acceptable salt of claim 164.

166. The crystalline form of claim 165, characterized by:
an X-ray powder diffraction pattern substantially as shown in FIG. 1; or
an X-ray powder diffraction pattern comprising a peak at 16.3 degrees±0.2 degrees 2-theta, on the 2-theta scale with lambda=1.54 angstroms (Cu Kα); or
an X-ray powder diffraction pattern comprising peaks at 6.7, 11.0, and 16.3 degrees±0.2 degrees 2-theta, on the 2-theta scale with lambda=1.54 angstroms (Cu Kα); or
an X-ray powder diffraction pattern comprising peaks at 6.7, 16.3, 20.4, and 30.7 degrees±0.2 degree 2-theta, on the 2-theta scale with lambda=1.54 angstroms (Cu Kα); or
an X-ray powder diffraction pattern comprising peaks at 6.7, 11.0, 14.9, 16.3, 16.8, 20.4, 25.4 degrees±0.2 degree 2-theta, on the 2-theta scale with lambda=1.54 angstroms (Cu Kα); or
an X-ray powder diffraction pattern comprising peaks at three or more of 6.7, 11.0, 14.9, 16.3, 16.8, 20.4, 25.4, 25.8, 27.9, 29.1, and 30.7 degrees±0.2 degrees 2-theta, on the 2-theta scale with lambda=1.54 angstroms (Cu Kα); or
a differential scanning calorimetry (DSC) thermogram substantially as shown in FIG. 3 when heated at a rate of 10° C./min; or
a differential scanning calorimetry (DSC) thermogram comprising an endothermic peak at about 207° C. when heated at a rate of 10° C./min; or
a thermogravimetric analysis profile substantially as shown in FIG. 4 when heated at a rate of 20° C./min.

167. The crystalline form of claim 165, characterized by:
an X-ray powder diffraction pattern substantially as shown in FIG. 2; or
an X-ray powder diffraction pattern comprising a peak at 14.6 degrees±0.2 degrees 2-theta, on the 2-theta scale with lambda=1.54 angstroms (Cu Kα); or
an X-ray powder diffraction pattern comprising peaks at 13.0, 14.6, and 16.3 degrees±0.2 degrees 2-theta, on the 2-theta scale with lambda=1.54 angstroms (Cu Kα); or
an X-ray powder diffraction pattern comprising peaks at 8.3, 13.0, 14.6, 16.3, 26.3, and 27.0 degrees±0.2 degree 2-theta, on the 2-theta scale with lambda=1.54 angstroms (Cu Kα); or
an X-ray powder diffraction pattern comprising peaks at 8.3, 13.0, 14.6, 15.3, 16.3, 16.7, 27.0, and 27.2 degrees±0.2 degree 2-theta, on the 2-theta scale with lambda=1.54 angstroms (Cu Kα); or
an X-ray powder diffraction pattern comprising peaks at three or more of 3.1, 8.3, 13.0, 14.6, 15.3, 16.3, 16.7, 18.4, 26.3, 26.5, 27.0, and 27.2 degrees±0.2 degrees 2-theta, on the 2-theta scale with lambda=1.54 angstroms (Cu Kα); or
a differential scanning calorimetry (DSC) thermogram substantially as shown in FIG. 5 when heated at a rate of 10° K/min; or
a differential scanning calorimetry (DSC) thermogram comprising an endothermic peak at about 185° C. when heated at a rate of 10° K/min; or
a thermogravimetric analysis profile substantially as shown in FIG. 5 when heated at a rate of 10° K/min.

168. The crystalline form of claim 165, characterized by an X-ray powder diffraction pattern substantially as shown in FIG. 14; or a differential scanning calorimetry (DSC) thermogram substantially as shown in FIG. 15 when heated at a rate of 10° C./min; or a thermogravimetric analysis profile substantially as shown in FIG. 16 when heated at a rate of 20° C./min.

169. The pharmaceutically acceptable salt of claim 163, wherein the salt is the hydrochloride salt having Formula IB

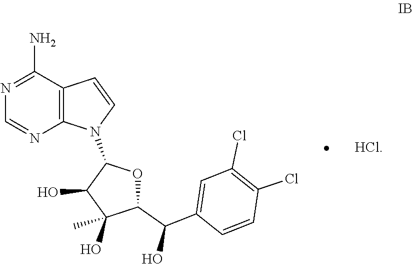 [SMILES: C[C@@]1(O)[C@@H]([C@H](O)c2ccc(Cl)c(Cl)c2)O[C@@H](n2ccc3c(N)ncnc32)[C@@H]1O ; "HCl."]

170. A crystalline form of the pharmaceutically acceptable salt of claim 169.

171. The crystalline form of claim 170, characterized by:
an X-ray powder diffraction pattern substantially as shown in FIG. 6; or
an X-ray powder diffraction pattern comprising a peak at 5.4 degrees±0.2 degrees 2-theta, on the 2-theta scale with lambda=1.54 angstroms (Cu Kα); or
an X-ray powder diffraction pattern comprising peaks at 5.4, 10.9, and 16.4 degrees±0.2 degrees 2-theta, on the 2-theta scale with lambda=1.54 angstroms (Cu Kα); or
an X-ray powder diffraction pattern comprising peaks at 5.4, 10.9, 21.2, and 24.2 degrees±0.2 degree 2-theta, on the 2-theta scale with lambda=1.54 angstroms (Cu Kα); or
an X-ray powder diffraction pattern comprising peaks at 5.4, 10.9, 16.4, 21.2, and 24.2 degrees±0.2 degree 2-theta, on the 2-theta scale with lambda=1.54 angstroms (Cu Kα); or
an X-ray powder diffraction pattern comprising peaks at three or more of 5.4, 10.9, 16.4, 21.2, 24.2, and 27.5 degrees±0.2 degrees 2-theta, on the 2-theta scale with lambda=1.54 angstroms (Cu Kα); or
a differential scanning calorimetry (DSC) thermogram substantially as shown in FIG. 9 when heated at a rate of 10° C./min; or
a differential scanning calorimetry (DSC) thermogram comprising an endothermic peak at about 268° C. when heated at a rate of 10° C./min; or
a thermogravimetric analysis profile substantially as shown in FIG. 10 when heated at a rate of 20° C./min.

172. The crystalline form of claim 170, characterized by:
an X-ray powder diffraction pattern substantially as shown in FIG. 7; or
an X-ray powder diffraction pattern comprising a peak at 5.0 degrees±0.2 degrees 2-theta, on the 2-theta scale with lambda=1.54 angstroms (Cu Kα); or
an X-ray powder diffraction pattern comprising peaks at 5.0, 15.2, and 24.3 degrees±0.2 degrees 2-theta, on the 2-theta scale with lambda=1.54 angstroms (Cu Kα); or
an X-ray powder diffraction pattern comprising peaks at 5.0, 15.2, 24.3, and 30.8 degrees±0.2 degree 2-theta, on the 2-theta scale with lambda=1.54 angstroms (Cu Kα); or
an X-ray powder diffraction pattern comprising peaks at 5.0, 10.1, 13.7, 15.2, 17.1, 24.3, and 30.8 degrees±0.2 degree 2-theta, on the 2-theta scale with lambda=1.54 angstroms (Cu Kα).

173. The crystalline form of claim 170, characterized by:
an X-ray powder diffraction pattern substantially as shown in FIG. 8; or
an X-ray powder diffraction pattern comprising a peak at 11.4 degrees±0.2 degrees 2-theta, on the 2-theta scale with lambda=1.54 angstroms (Cu Kα); or
an X-ray powder diffraction pattern comprising peaks at 11.4, 11.6, 15.1, and 16.7 degrees±0.2 degrees 2-theta, on the 2-theta scale with lambda=1.54 angstroms (Cu Kα); or
an X-ray powder diffraction pattern comprising peaks at 4.9, 11.4, 11.6, 15.1, and 16.7 degrees±0.2 degree 2-theta, on the 2-theta scale with lambda=1.54 angstroms (Cu Kα); or
an X-ray powder diffraction pattern comprising peaks at 4.9, 11.4, 11.6, 15.1, 16.7, 21.0, and 22.4 degrees±0.2 degree 2-theta, on the 2-theta scale with lambda=1.54 angstroms (Cu Kα); or
an X-ray powder diffraction pattern comprising peaks at three or more of 4.9, 7.1, 11.4, 11.6, 12.4, 13.6, 14.3, 15.1, 16.5, 16.7, 16.9, 17.0, 20.3, 21.0, 22.4, 23.0, 23.5, and 23.8 degrees±0.2 degrees 2-theta, on the 2-theta scale with lambda=1.54 angstroms (Cu Kα); or
a differential scanning calorimetry (DSC) thermogram substantially as shown in FIG. 11 when heated at a rate of 10° C./min; or
a differential scanning calorimetry (DSC) thermogram comprising an endothermic peak at about 196° C. when heated at a rate of 10° C./min; or
a thermogravimetric analysis profile substantially as shown in FIG. 11 when heated at a rate of 10° C./min.

174. The crystalline form of claim 170, characterized by:
an X-ray powder diffraction pattern substantially as shown in FIG. 17; or
an X-ray powder diffraction pattern comprising a peak at 5.3 and 15.5 degrees±0.2 degrees 2-theta, on the 2-theta scale with lambda=1.54 angstroms (Cu Kα); or
an X-ray powder diffraction pattern comprising peaks at 15.5 and 31.0 degrees±0.2 degrees 2-theta, on the 2-theta scale with lambda=1.54 angstroms (Cu Kα); or
an X-ray powder diffraction pattern comprising peaks at 15.5 and 24.5 degrees±0.2 degree 2-theta, on the 2-theta scale with lambda=1.54 angstroms (Cu Kα); or
an X-ray powder diffraction pattern comprising peaks at 5.3, 15.5, 17.3, 24.5, and 31.0 degrees±0.2 degree 2-theta, on the 2-theta scale with lambda=1.54 angstroms (Cu Kα); or
an X-ray powder diffraction pattern comprising peaks at three or more of 5.3, 15.5, 17.3, 21.5, 24.5, 28.0, and 31.0 degrees±0.2 degrees 2-theta, on the 2-theta scale with lambda=1.54 angstroms (Cu Kα); or
a differential scanning calorimetry (DSC) thermogram substantially as shown in FIG. 18 when heated at a rate of 10° C./min; or
a differential scanning calorimetry (DSC) thermogram comprising an endothermic peak at about 188° C. when heated at a rate of 10° C./min; or
a differential scanning calorimetry (DSC) thermogram comprising an endothermic peak at about 267° C. when heated at a rate of 10° C./min; or
a thermogravimetric analysis profile substantially as shown in FIG. 19 when heated at a rate of 20° C./min.

175. The crystalline form of claim 170, characterized by:
an X-ray powder diffraction pattern substantially as shown in FIG. 20; or
an X-ray powder diffraction pattern comprising a peak at 13.2 and 17.5 degrees±0.2 degrees 2-theta, on the 2-theta scale with lambda=1.54 angstroms (Cu Kα); or
an X-ray powder diffraction pattern comprising peaks at 13.2, 17.5, 26.3, and 28.3 degrees±0.2 degrees 2-theta, on the 2-theta scale with lambda=1.54 angstroms (Cu Kα); or
an X-ray powder diffraction pattern comprising peaks at 13.2, 17.5, 18.8, 19.5, and 20.2 degrees±0.2 degree 2-theta, on the 2-theta scale with lambda=1.54 angstroms (Cu Kα); or
an X-ray powder diffraction pattern comprising peaks at 13.2, 17.5, 24.9, 26.3, and 28.3 degrees±0.2 degree 2-theta, on the 2-theta scale with lambda=1.54 angstroms (Cu Kα); or
an X-ray powder diffraction pattern comprising peaks at three or more of 13.2, 17.5, 18.8, 19.5, 20.2, 24.9, 26.3, and 28.3 degrees±0.2 degrees 2-theta, on the 2-theta scale with lambda=1.54 angstroms (Cu Kα); or
a differential scanning calorimetry (DSC) thermogram substantially as shown in FIG. 21 when heated at a rate of 10° C./min; or
a differential scanning calorimetry (DSC) thermogram comprising an endothermic peak at about 271° C. when heated at a rate of 10° C./min; or
a thermogravimetric analysis profile substantially as shown in FIG. 22 when heated at a rate of 20° C./min.

176. The crystalline form of claim 170, characterized by:
an X-ray powder diffraction pattern substantially as shown in FIG. 26; or
an X-ray powder diffraction pattern comprising a peak at 16.1 and 25.0 degrees±0.2 degrees 2-theta, on the 2-theta scale with lambda=1.54 angstroms (Cu Kα); or
an X-ray powder diffraction pattern comprising peaks at 14.3, 16.1, 17.4, and 21.9 degrees±0.2 degrees 2-theta, on the 2-theta scale with lambda=1.54 angstroms (Cu Kα); or
an X-ray powder diffraction pattern comprising peaks at 14.3, 16.1, 17.4, 21.9, and 25.0 degrees±0.2 degree 2-theta, on the 2-theta scale with lambda=1.54 angstroms (Cu Kα); or
an X-ray powder diffraction pattern comprising peaks at 14.3, 16.1, 17.4, 21.9, 25.0, and 26.9 degrees±0.2 degree 2-theta, on the 2-theta scale with lambda=1.54 angstroms (Cu Kα); or
an X-ray powder diffraction pattern comprising peaks at three or more of 14.3, 16.1, 17.4, 21.9, 25.0, 26.9, and 32.3 degrees±0.2 degrees 2-theta, on the 2-theta scale with lambda=1.54 angstroms (Cu Kα); or
a differential scanning calorimetry (DSC) thermogram substantially as shown in FIG. 27 when heated at a rate of 10° C./min; or
a differential scanning calorimetry (DSC) thermogram comprising an endothermic peak at about 270° C. when heated at a rate of 10° C./min; or
a thermogravimetric analysis profile substantially as shown in FIG. 28 when heated at a rate of 20° C./min.

177. The crystalline form of claim 170, characterized by:
an X-ray powder diffraction pattern substantially as shown in FIG. 32; or
an X-ray powder diffraction pattern comprising peaks at 15.7, 24.6, and 31.3 degrees±0.2 degrees 2-theta, on the 2-theta scale with lambda=1.54 angstroms (Cu Kα); or
an X-ray powder diffraction pattern comprising peaks at 15.7, 17.3, 24.6, and 31.3 degrees±0.2 degrees 2-theta, on the 2-theta scale with lambda=1.54 angstroms (Cu Kα); or
an X-ray powder diffraction pattern comprising peaks at 15.7, 17.3, 21.7, 24.6, and 31.3 degrees±0.2 degree 2-theta, on the 2-theta scale with lambda=1.54 angstroms (Cu Kα); or
an X-ray powder diffraction pattern comprising peaks at 15.7, 17.3, 21.7, 24.6, 26.1, 28.2, and 31.3 degrees±0.2 degree 2-theta, on the 2-theta scale with lambda=1.54 angstroms (Cu Kα); or
an X-ray powder diffraction pattern comprising peaks at three or more of 5.4, 15.7, 17.3, 21.7, 24.6, 26.1, 28.2, and 31.3 degrees±0.2 degrees 2-theta, on the 2-theta scale with lambda=1.54 angstroms (Cu Kα); or
a differential scanning calorimetry (DSC) thermogram substantially as shown in FIG. 33 when heated at a rate of 10° C./min; or
a differential scanning calorimetry (DSC) thermogram comprising an endothermic peak at about 208° C. when heated at a rate of 10° C./min; or
a thermogravimetric analysis profile substantially as shown in FIG. 34 when heated at a rate of 20° C./min.

178. The crystalline form of claim 170, characterized by:
an X-ray powder diffraction pattern substantially as shown in FIG. 38; or
an X-ray powder diffraction pattern comprising peaks at 15.9, 21.5, and 24.5 degrees±0.2 degrees 2-theta, on the 2-theta scale with lambda=1.54 angstroms (Cu Kα); or
an X-ray powder diffraction pattern comprising peaks at 15.5, 15.9, 16.7, 17.5, and 21.5 degrees±0.2 degrees 2-theta, on the 2-theta scale with lambda=1.54 angstroms (Cu Kα); or
an X-ray powder diffraction pattern comprising peaks at 15.5, 15.9, 16.7, 17.5, 21.5, 23.0, and 24.5 degrees±0.2 degree 2-theta, on the 2-theta scale with lambda=1.54 angstroms (Cu Kα); or
an X-ray powder diffraction pattern comprising peaks at 13.1, 15.5, 15.9, 16.7, 17.5, 21.5, 23.0, 24.5, and 28.3 degrees±0.2 degree 2-theta, on the 2-theta scale with lambda=1.54 angstroms (Cu Kα); or
an X-ray powder diffraction pattern comprising peaks at three or more of 13.1, 15.5, 15.9, 16.7, 17.5, 21.5, 23.0, 24.5, 28.3, and 29.0 degrees±0.2 degrees 2-theta, on the 2-theta scale with lambda=1.54 angstroms (Cu Kα); or
a differential scanning calorimetry (DSC) thermogram substantially as shown in FIG. 39 when heated at a rate of 10° C./min; or
a differential scanning calorimetry (DSC) thermogram comprising an endothermic peak at about 221° C. when heated at a rate of 10° C./min; or
a thermogravimetric analysis profile substantially as shown in FIG. 40 when heated at a rate of 20° C./min.

179. The crystalline form of claim 170, characterized by:
an X-ray powder diffraction pattern substantially as shown in FIG. 42; or
an X-ray powder diffraction pattern comprising peaks at 15.6 and 24.6 degrees±0.2 degrees 2-theta, on the 2-theta scale with lambda=1.54 angstroms (Cu Kα); or
an X-ray powder diffraction pattern comprising peaks at 15.6, 17.4, and 21.6 degrees±0.2 degrees 2-theta, on the 2-theta scale with lambda=1.54 angstroms (Cu Kα); or
an X-ray powder diffraction pattern comprising peaks at 15.6, 17.4, 21.6, and 24.6 degrees±0.2 degree 2-theta, on the 2-theta scale with lambda=1.54 angstroms (Cu Kα); or
an X-ray powder diffraction pattern comprising peaks at 14.1, 15.6, 17.4, 21.6, and 24.6 degrees±0.2 degree 2-theta, on the 2-theta scale with lambda=1.54 angstroms (Cu Kα); or
an X-ray powder diffraction pattern comprising peaks at three or more of 5.3, 14.1, 15.6, 17.4, 21.6, and 24.6 degrees±0.2 degrees 2-theta, on the 2-theta scale with lambda=1.54 angstroms (Cu Kα); or
a differential scanning calorimetry (DSC) thermogram substantially as shown in FIG. 43 when heated at a rate of 10° C./min; or
a differential scanning calorimetry (DSC) thermogram comprising an endothermic peak at about 188° C. when heated at a rate of 10° C./min; or
a differential scanning calorimetry (DSC) thermogram comprising an endothermic peak at about 271° C. when heated at a rate of 10° C./min; or
a thermogravimetric analysis profile substantially as shown in FIG. 44 when heated at a rate of 20° C./min.

180. The pharmaceutically acceptable salt of claim 163, wherein the salt is the oxalate salt having Formula IC

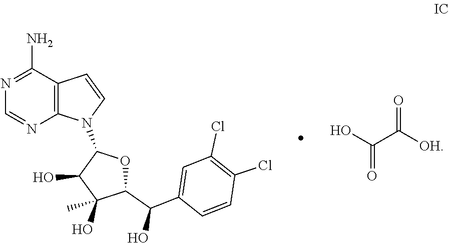 [SMILES: C[C@@]1(O)[C@@H]([C@H](O)c2ccc(Cl)c(Cl)c2)O[C@@H](n2ccc3c(N)ncnc32)[C@@H]1O ; O=C(O)C(=O)O]

181. A crystalline form of the pharmaceutically acceptable salt of claim 180.

182. The crystalline form of claim 181, characterized by:
an X-ray powder diffraction pattern substantially as shown in FIG. 12; or
an X-ray powder diffraction pattern comprising a peak at 10.5 degrees±0.2 degrees 2-theta, on the 2-theta scale with lambda=1.54 angstroms (Cu Kα); or
an X-ray powder diffraction pattern comprising peaks at 10.5, 14.7, and 16.2 degrees±0.2 degrees 2-theta, on the 2-theta scale with lambda=1.54 angstroms (Cu Kα); or
an X-ray powder diffraction pattern comprising peaks at 10.5, 14.7, 16.2, and 28.7 degrees±0.2 degree 2-theta, on the 2-theta scale with lambda=1.54 angstroms (Cu Kα); or
an X-ray powder diffraction pattern comprising peaks at 10.5, 14.7, 16.2, 17.6, 17.7, 19.6, 28.7, and 28.9 degrees±0.2 degree 2-theta, on the 2-theta scale with lambda=1.54 angstroms (Cu Kα); or
an X-ray powder diffraction pattern comprising peaks at three or more of 10.5, 11.6, 13.1, 14.2, 14.7, 14.9, 16.2, 17.6, 17.7, 19.6, 28.7, and 28.9 degrees±0.2 degrees 2-theta, on the 2-theta scale with lambda=1.54 angstroms (Cu Kα).

183. The pharmaceutically acceptable salt of claim 163, wherein the salt is the phosphate salt having Formula ID

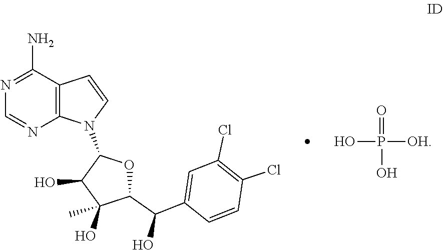 [SMILES: C[C@@]1(O)[C@@H]([C@H](O)c2ccc(Cl)c(Cl)c2)O[C@@H](n2ccc3c(N)ncnc32)[C@@H]1O ; O=P(O)(O)O]

184. A crystalline form of the pharmaceutically acceptable salt of claim 183.

185. The crystalline form of claim 184, characterized by:
an X-ray powder diffraction pattern substantially as shown in FIG. 13; or
an X-ray powder diffraction pattern comprising a peak at 3.6 degrees±0.2 degrees 2-theta, on the 2-theta scale with lambda=1.54 angstroms (Cu Kα); or
an X-ray powder diffraction pattern comprising peaks at 3.6, and 10.7 degrees±0.2 degrees 2-theta, on the 2-theta scale with lambda=1.54 angstroms (Cu Kα); or
an X-ray powder diffraction pattern comprising peaks at 3.6, 10.7, and 15.6 degrees±0.2 degree 2-theta, on the 2-theta scale with lambda=1.54 angstroms (Cu Kα); or
an X-ray powder diffraction pattern comprising peaks at three or more of 3.6, 10.7, 15.6, 17.9, and 18.7 degrees±0.2 degrees 2-theta, on the 2-theta scale with lambda=1.54 angstroms (Cu Kα).

186. The crystalline form of claim 184, characterized by:
an X-ray powder diffraction pattern substantially as shown in FIG. 45; or
an X-ray powder diffraction pattern comprising peaks at 18.1, 20.0, 26.2, and 28.1 degrees±0.2 degrees 2-theta, on the 2-theta scale with lambda=1.54 angstroms (Cu Kα); or
an X-ray powder diffraction pattern comprising peaks at 17.1, 18.1, 20.0, 26.2, and 28.1 degrees±0.2 degrees 2-theta, on the 2-theta scale with lambda=1.54 angstroms (Cu Kα); or
an X-ray powder diffraction pattern comprising peaks at 10.6, 17.1, 18.1, 20.0, 26.2, and 28.1 degrees±0.2 degree 2-theta, on the 2-theta scale with lambda=1.54 angstroms (Cu Kα); or
an X-ray powder diffraction pattern comprising peaks at three or more of 10.6, 17.1, 18.1, 20.0, 21.5, 22.4, 26.2, and 28.1 degrees±0.2 degrees 2-theta, on the 2-theta scale with lambda=1.54 angstroms (Cu Kα); or
a differential scanning calorimetry (DSC) thermogram substantially as shown in FIG. 46 when heated at a rate of 10° C./min; or
a differential scanning calorimetry (DSC) thermogram comprising an endothermic peak at about 161° C. when heated at a rate of 10° C./min; or
a differential scanning calorimetry (DSC) thermogram comprising an endothermic peak at about 221° C. when heated at a rate of 10° C./min; or
a thermogravimetric analysis profile substantially as shown in FIG. 47 when heated at a rate of 20° C./min.

187. The pharmaceutically acceptable salt of claim 163, wherein the salt is the bisulfate salt having Formula IE

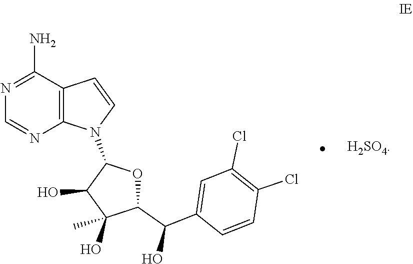 [SMILES: C[C@@]1(O)[C@@H]([C@H](O)c2ccc(Cl)c(Cl)c2)O[C@@H](n2ccc3c(N)ncnc32)[C@@H]1O ; "H2SO4."]

188. A crystalline form of the pharmaceutically acceptable salt of claim 187.

189. A crystalline form of the compound of Formula I:

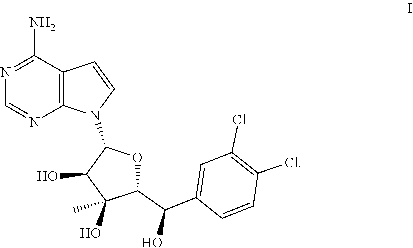 [SMILES: C[C@@]1(O)[C@@H]([C@H](O)c2ccc(Cl)c(Cl)c2)O[C@@H](n2ccc3c(N)ncnc32)[C@@H]1O]

190. The crystalline form of claim 189, characterized by:
an X-ray powder diffraction pattern substantially as shown in FIG. 48; or
an X-ray powder diffraction pattern comprising peaks at 17.3, and 18.1 degrees±0.2 degrees 2-theta, on the 2-theta scale with lambda=1.54 angstroms (Cu Kα); or
an X-ray powder diffraction pattern comprising peaks at 17.3, 18.1, 25.2, and 27.1 degrees±0.2 degrees 2-theta, on the 2-theta scale with lambda=1.54 angstroms (Cu Kα); or
an X-ray powder diffraction pattern comprising peaks at 17.3, 18.1, 25.2, 27.1, 28.3, 28.8, and 30.0 degrees±0.2 degree 2-theta, on the 2-theta scale with lambda=1.54 angstroms (Cu Kα); or
an X-ray powder diffraction pattern comprising peaks at 17.3, 18.1, 20.4, 24.2, 25.2, 27.1, 28.3, 28.8, and 30.0 degrees±0.2 degree 2-theta, on the 2-theta scale with lambda=1.54 angstroms (Cu Kα); or
an X-ray powder diffraction pattern comprising peaks at three or more of 15.0, 17.3, 18.1, 20.4, 24.2, 25.2, 27.1, 28.3, 28.8, and 30.0 degrees±0.2 degrees 2-theta, on the 2-theta scale with lambda=1.54 angstroms (Cu Kα); or
a differential scanning calorimetry (DSC) thermogram substantially as shown in FIG. 49 when heated at a rate of 10° C./min; or
a differential scanning calorimetry (DSC) thermogram comprising an endothermic peak at about 140° C. when heated at a rate of 10° C./min; or
a thermogravimetric analysis profile substantially as shown in FIG. 50 when heated at a rate of 20° C./min.

191. The crystalline form of claim 189, characterized by:
an X-ray powder diffraction pattern substantially as shown in FIG. 54; or
an X-ray powder diffraction pattern comprising a peak at 23.5 and 24.9 degrees±0.2 degrees 2-theta, on the 2-theta scale with lambda=1.54 angstroms (Cu Kα); or
an X-ray powder diffraction pattern comprising peaks at 18.9, 23.5, 24.3, and 24.9 degrees±0.2 degrees 2-theta, on the 2-theta scale with lambda=1.54 angstroms (Cu Kα); or
an X-ray powder diffraction pattern comprising peaks at 15.1, 17.4, 18.9, 23.5, 24.3, and 24.9 degrees±0.2 degree 2-theta, on the 2-theta scale with lambda=1.54 angstroms (Cu Kα); or
an X-ray powder diffraction pattern comprising peaks at 15.1, 17.4, 18.9, 23.5, 24.3, 24.9, and 25.5 degrees±0.2 degree 2-theta, on the 2-theta scale with lambda=1.54 angstroms (Cu Kα); or
an X-ray powder diffraction pattern comprising peaks at three or more of 15.1, 17.4, 18.9, 23.5, 24.3, 24.9, 25.5, and 30.3 degrees±0.2 degrees 2-theta, on the 2-theta scale with lambda=1.54 angstroms (Cu Kα); or
a differential scanning calorimetry (DSC) thermogram substantially as shown in FIG. 55 when heated at a rate of 10° C./min; or
a differential scanning calorimetry (DSC) thermogram comprising an endothermic peak at about 137° C. when heated at a rate of 10° C./min.

192. The crystalline form of claim 189, characterized by:
an X-ray powder diffraction pattern substantially as shown in FIG. 56; or
an X-ray powder diffraction pattern comprising peaks at 16.6, and 17.4 degrees±0.2 degrees 2-theta, on the 2-theta scale with lambda=1.54 angstroms (Cu Kα); or
an X-ray powder diffraction pattern comprising peaks at 17.4, 20.4, and 25.8 degrees±0.2 degrees 2-theta, on the 2-theta scale with lambda=1.54 angstroms (Cu Kα); or
an X-ray powder diffraction pattern comprising peaks at 17.4, 20.4, 24.9, 25.8, and 26.3 degrees±0.2 degree 2-theta, on the 2-theta scale with lambda=1.54 angstroms (Cu Kα); or
an X-ray powder diffraction pattern comprising peaks at 16.6, 17.4, 20.4, 24.9, 25.8, 26.3, and 27.7 degrees±0.2 degree 2-theta, on the 2-theta scale with lambda=1.54 angstroms (Cu Kα); or
an X-ray powder diffraction pattern comprising peaks at three or more of 9.2, 16.6, 17.4, 20.4, 24.9, 25.8, 26.3, 27.7, and 41.5 degrees±0.2 degrees 2-theta, on the 2-theta scale with lambda=1.54 angstroms (Cu Kα); or
a differential scanning calorimetry (DSC) thermogram substantially as shown in FIG. 57 when heated at a rate of 10° C./min; or
a differential scanning calorimetry (DSC) thermogram comprising an endothermic peak at about 125° C. when heated at a rate of 10° C./min.

193. A pharmaceutical composition comprising a compound according to claim 163, and a pharmaceutically acceptable excipient.

194. A method of inhibiting a protein arginine methyltransferase 5 (PRMT5) enzyme, comprising: contacting the PRMT5 enzyme with an effective amount of a compound of claim 163.

195. A method of treating a disease or disorder associated with aberrant PRMT5 activity in a subject comprising administering to the subject, a compound of claim 163.

196. The method of claim 195, wherein the disease or disorder associated with aberrant PRMT5 activity is adenoid cystic carcinoma (ACC), breast cancer, lung cancer, pancreatic cancer, prostate cancer, colon cancer, ovarian cancer, uterine cancer, cervical cancer, leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CIVIL), mastocytosis, chronic lymphocytic leukemia (CLL), multiple myeloma (MM), myelodysplastic syndrome (MDS), epidermoid cancer, or hemoglobinopathies such as b-thalassemia and sickle cell disease (SCD).

197. The method of claim 195, wherein the compound is administered in combination with one or more other agents.

198. A pharmaceutical composition comprising a compound according to claim 189, and a pharmaceutically acceptable excipient.

199. A method of inhibiting a protein arginine methyltransferase 5 (PRA/ITS) enzyme, comprising: contacting the PRMT5 enzyme with an effective amount of a compound of claim 189.

200. A method of treating a disease or disorder associated with aberrant PRMT5 activity in a subject comprising administering to the subject, a compound of claim 189.

201. The method of claim 200, wherein the disease or disorder associated with aberrant PRMT5 activity is adenoid cystic carcinoma (ACC), breast cancer, lung cancer, pancreatic cancer, prostate cancer, colon cancer, ovarian cancer, uterine cancer, cervical cancer, leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CIVIL), mastocytosis, chronic lymphocytic leukemia (CLL), multiple myeloma (MM), myelodysplastic syndrome (MDS), epidermoid cancer, or hemoglobinopathies such as b-thalassemia and sickle cell disease (SCD).

202. The method of claim 201, wherein the compound is administered in combination with one or more other agents.
